(19) United States
(12) Patent Application Publication
(10) Pub. No.: US 2018/0031562 A1
(43) Pub. Date: Feb. 1, 2018

(54) CANCER BIOMARKERS

(71) Applicant: OSLO UNIVERSITETSSYKEHUS HF, Oslo (NO)

(72) Inventors: Alicia LLORENTE, Oslo (NO); Tore SKOTLAND, Nittedal (NO); Kirsten SANDVIG, Nittedal (NO); Anders ØVERBYE, Ski (NO)

(21) Appl. No.: 15/542,873

(22) PCT Filed: Jan. 14, 2016
(86) PCT No.: PCT/EP2016/050692

§ 371 Date: Jul. 11, 2017

(30) Foreign Application Priority Data
Jan. 14, 2015 (GB) 1500584.6

(51) Int. Cl. G01N 33/574

(52) U.S. Cl. CPC G01N 33/57434

(57) ABSTRACT

The present invention relates to methods for screening for prostate cancer in a subject. In particular, the present invention provides a method of for screening for prostate cancer in a subject, said method comprising determining the level of one or more of certain polypeptides in a urinary exosome-containing sample that has been obtained from a subject. Such methods can be used to for diagnosing prostate cancer, for the prognosis of prostate cancer, for monitoring the progression of prostate cancer in a subject, for determining the clinical severity of prostate cancer, for predicting the response of a subject to therapy, or for determining the efficacy of a therapeutic regime being used to treat prostate cancer.

The present invention relates generally to prostate cancer biomarkers and to methods of screening for prostate cancer. Such methods involve determining the level of certain biomarkers which are indicative of prostate cancer in a subject.

Prostate cancer is a global health problem. It represents 12% of all cancer cases worldwide, and it is the second most commonly diagnosed cancer in men (Baade P D, Youlden D R, & Krnjacki L J (2009) International epidemiology of prostate cancer: geographical distribution and secular trends. Mol Nutr. Food Res., 53, 171-184). Prostate specific antigen (PSA) has been used for nearly three decades as a biomarker for prostate cancer and is still a useful marker for prostate cancer after diagnosis. However, the serum PSA test lacks sensitivity and specificity, and this has resulted in prostate cancer overdiagnosis and overtreatment (Welch H G & Albertsen P C (2009) Prostate cancer diagnosis and treatment after the introduction of prostate-specific antigen screening: 1986-2005. J. Natl. Cancer Inst., 101, 1325-1329). Recently, the U.S. Preventive Services Task force decided to recommend against the use of this biomarker for prostate cancer screening (Moyer V A (2012) Screening for prostate cancer: U.S. Preventive Services Task Force recommendation statement. Ann. Intern. Med., 157, 120-134).

PSA also suffers from a high rate of false negatives, as it has been reported that as many as 15% of patients with clinically significant prostate cancer (Thompson et al., 2003, New England Journal of Medicine; 349(3): 215-224) had normal PSA levels. Thus, the PSA test is inadequate with respect to both sensitivity and specificity. This illustrates the need for a diagnostic test that would reduce the number of both false positives and false negatives and improve early diagnosis.

A rise in PSA levels combined with a positive digital rectal exam (DRE) typically leads to referral of the patient to a urologist for a biopsy to confirm diagnosis of prostate cancer, as well as determine its grade. Since it is easy to miss a small cancer tissue within the prostate consisting of otherwise healthy tissue, many samples from different regions of the prostate are typically collected at each biopsy procedure. Nevertheless, sampling errors can still result in cancer being missed in up to 25% of cases, necessitating repeated biopsy procedures in case of negative results, with the associated discomforts and risks. There is thus also a need for supplementary non-invasive tests that may be administered after a negative biopsy to determine the need for repeat biopsies.

Examination of biopsies by a pathologist is used to determine the grade (Gleason score) of the cancer. The Gleason score is used in combination with information regarding the localization of the tumor within and around the prostate to determine the Stage I-IV, where IV is the most aggressive. Following diagnosis of prostate cancer, management decisions are currently based on numerous risk stratification systems that are generally based on different threshold and weighting of three key parameters to indicate high-risk disease: PSA levels, Gleason score, and clinical stage of the disease. Various classification guidelines based on these parameters are in existence, and may give drastically different results, leading to possible over- or under-treatment (Buck & Chughtai, 2014, B. Nat. Rev. Urol. 11:256-257). There is thus also a clear unmet need for biomarkers that may improve risk stratification.

Since prostate cancer is in many cases a slowly progressing disease, it is increasingly recommended that very low-risk patients do not immediately seek treatment (National Comprehensive Cancer Network guidelines, 2012), but are subject to watchful waiting or active surveillance. Active surveillance requires frequent testing involving biopsies, which are invasive, and PSA screening, which is often utilized, although its benefits and accuracy are both hotly disputed. With better monitoring tools, patients and doctors will be more comfortable choosing conservative management and postponing treatment.

What is needed in the art are new methods of screening for prostate cancer. Such methods may be useful for assessing whether a subject qualifies for a first biopsy, reducing false negative biopsies (decision on whether to perform additional biopsies), distinguishing between indolent and aggressive cancer (decision between active surveillance and treatment), and monitoring of patients under active surveillance. Preferably such methods would be non-invasive and performed on readily obtainable samples. The identification of novel biomarkers for prostate cancer may potentially have clinical implications for a large number of patients.

The present inventors have identified certain polypeptides (proteins) that are differentially expressed in urinary exosomes from prostate cancer patients in comparison to control subjects. These differentially expressed polypeptides act as biomarkers for prostate cancer and thus are useful in screening for prostate cancer in subjects. Such biomarkers may also be used in methods of assessing whether or not a subject qualifies for first biopsy, reducing false negative biopsies (decision on whether to perform additional biopsies), distinguishing between indolent and aggressive cancer (decision between active surveillance and treatment), and monitoring of patients under active surveillance.

Thus, in one aspect the present invention provides a method of screening for prostate cancer in a subject, said method comprising

determining the level in a sample of one or more polypeptides selected from the group consisting of:
Transmembrane protein 256, Adipogenesis regulatory factor, Ragulator complex protein LAMTOR1, Plastin-2, Ras-related protein Rab-2A, Ras-related protein Rab-3B, Ras-related protein Rab-3D, Ras-related protein Rab-7a, V-type proton ATPase 16 kDa proteolipid subunit, Metalloreductase STEAP4, Protein DJ-1, Protein S100-P, Synaptotagmin-like protein 4, ADP-ribosylation factor-like protein 8B, Proton myo-inositol cotransporter, Ras-related protein Rab-6A, Tetraspanin-6, Claudin-10, Claudin-2, Claudin-3, GDP-mannose 4.6 dehydratase, Glucosamine-6-phosphate isomerase 1, Lysosome membrane protein 2, Major facilitator superfamily domain-containing protein 12, Melanophilin, Sepiapterin reductase, Thioredoxin domain-containing protein 17, 3-hydroxybutyrate dehydrogenase type 2, Calmodulin, Carboxypeptidase Q, Flotillin-2, Galectin-3-binding protein, P2X purinoceptor 4, Protein dopey-2, Protein S100-A6, Ras-related protein Rab-35, Serine/threonine-protein phosphatase 2A catalytic subunit alpha iso form, 2′-deoxynucleoside 5′-phosphate N-hydrolase 1, Acid ceramidase, Calbindin, CD59 glycoprotein, CD81 antigen, Cytochrome b561, Enolase-phosphatase E1, Golgi phosphoprotein 3, Nicastrin, Probable serine carboxypeptidase CPVL, Ragulator complex protein LAMTORS, Ras-related protein Rab-27B, Secretory carrier-associated membrane protein 2, Spermine synthase, S-phase kinase-associated protein 1, Transmembrane 7 superfamily member 3, Tumor protein D52, Ubiquitin-conjugating enzyme E2 variant 2, UDP-glucose 6-dehydrogenase, Zinc-alpha-2-glycoprotein, Glycerophosphodiester phosphodiesterase domain-containing protein 3, EF-hand domain-containing protein D2, Ras-related protein Rab-14, Omega-amidase NIT2, Alpha-actinin-1, Monocarboxylate transporter 5, Ras-related protein Rab-12, Ras-related protein Rab-8A, Transmembrane protein 63A, Beta-2-microglobulin, V-type proton ATPase subunit d 1, Lipid phosphate phosphohydrolase 1, Integral membrane protein GPR155, 14-3-3 protein sigma, E3 ubiquitin-protein ligase LRSAM1, HLA class II histocompatibility antigen. DM alpha chain, Ras-related protein Rab-9A, Src substrate cortactin, Aquaporin-7, Gamma-synuclein, 14-3-3 protein theta, Aspartate aminotransferase. cytoplasmic, Chloride intracellular channel protein 3, Destrin, GTPase HRas, Prostaglandin reductase 2, T-complex protein 1 subunit epsilon, Inter-alpha-trypsin inhibitor heavy chain H4, Aldehyde dehydrogenase family 1 member A3, Annexin A3, Battenin, Cathepsin D, N(G).N(G)-dimethylarginine dimethylaminohydrolase 1, Neural proliferation differentiation and control protein 1, Proactivator polypeptide, Prostate-specific antigen, Protein lifeguard 3, Protein Niban, Protein spinster homolog 1, Ragulator complex protein LAMTOR2, Ragulator complex protein LAMTOR3, Synaptotagmin-7, Transmembrane protein 106B, Unconventional myosin-Vc, Vesicle-associated membrane protein 2, V-type proton ATPase subunit F, A-kinase anchor protein 7 isoforms alpha and beta, Arylsulfatase F, C-Jun-amino-terminal kinase-interacting protein 4, Deleted in malignant brain tumors 1 protein, Glia maturation factor beta, Glutamate carboxypeptidase 2, Glutathione synthetase, Hippocalcin-like protein 1, Myristoylated alanine-rich C-kinase substrate, NAD(P)H-hydrate epimerase, Napsin-A, Phosphoacetylglucosamine mutase, Probable phospholipid-transporting ATPase IA, Quinone oxidoreductase, Retinol-binding protein 5, Serine incorporator 2, Solute carrier family 15 member 1, Sorcin, Sucrase-isomaltase. Intestinal, Actin-related protein ⅔ complex subunit 4, Actin-related protein ⅔ complex subunit 5, Adenine phosphoribosyltransferase, Costars family protein ABRACL, Ig alpha-1 chain C region, Interferon-induced transmembrane protein 3, Lactotransferrin, Purine nucleoside phosphorylase, Syntaxin-binding protein 4, Tetraspanin-8, Vacuolar protein-sorting-associated protein 25, Voltage-dependent anion-selective channel protein 1, Collagen alpha-1(VI) chain, Signal transducing adapter molecule 1, CD9 antigen, Flotillin-1, Grancalcin, Mannose-1-phosphate guanyltransferase beta, Proteasome subunit alpha type-7, Ras-related protein Rab-18, Vacuolar protein sorting-associated protein 37C, Signal transducing adapter molecule 2, Pancreatic secretory granule membrane major glycoprotein GP2, 1-phosphatidylinosito14.5-bisphosphate phosphodiesterase gamma-2, Lysosome-associated membrane glycoprotein 2, Cysteine-rich C-terminal protein 1, Plasma serine protease inhibitor, Tyrosine-protein kinase Lck, Ragulator complex protein LAMTOR4, Secretory carrier-associated membrane protein 1, Peptidyl-prolyl cis-trans isomerase FKBP1A, Transmembrane protein 176A, Thymosin beta-4, Haloacid dehalogenase-like hydrolase domain-containing protein 2, Cell division control protein 42 homolog, Ras-related protein Rab-17, Chloride intracellular channel protein 6, Choline transporter-like protein 4, Flavin reductase (NADPH), Ras-related protein Rab-10, Heme-binding protein 2, Fatty acid-binding protein. epidermal, Small integral membrane protein 5, Lysosomal-associated transmembrane protein 4A, Phytanoyl-CoA dioxygenase domain-containing protein 1, Proteasome subunit alpha type-5, Calmodulin-like protein 3, Presenilin-1, Ribosyldihydronicotinamide dehydrogenase [quinone], Translationally-controlled tumor protein, Lysosome-associated membrane glycoprotein 1, ADP-ribosyl cyclase 1, Myotrophin, Dynein light chain 2. cytoplasmic, Dihydropteridine reductase, Nicotinate-nucleotide pyrophosphorylase [carboxylating], Cell cycle control protein 50A, Phosphomevalonate kinase, Eukaryotic translation initiation factor 4H, Protein tweety homo log 3, Sodium-dependent phosphate transport protein 2B, Ig lambda-2 chain C regions, Cellular retinoic acid-binding protein 2, Protein CutA, Proteasome subunit alpha type-4, Solute carrier family 35 member F6, Delta-aminolevulinic acid dehydratase, L-xylulose reductase, Uroplakin-1a, Cornifin-A, Zinc finger protein 185, Transmembrane protein 8A, Prenylcysteine oxidase 1, Lysozyme C, Paralemmin-1, Carcinoembryonic antigen-related cell adhesion molecule 6, Sodium/glucose cotransporter 1, Prostaglandin reductase 1, Protein S100-A9, MICAL-like protein 1, Alpha/beta hydrolase domain-containing protein 14B, Aquaporin-2, Glutathione S-transferase P, Probable almitoyltransferase ZDHHC1, Ras-related protein Rab-8B, Transmembrane protease serine 2, Ras-related protein Rab-1B, Ras-related protein Rab-1A, Ras-related protein Rab-43, Synaptogyrin-2, HLA class II histocompatibility antigen. DRB1-15 beta chain, Lysosomal protein NCU-G1, Calcium-binding protein 39, Dynamin-2, CDC42 small effector protein 2, Ferritin heavy chain, Solute carrier family 35 member F2, Probable hydrolase PNKD, Cathepsin Z, Tubulin beta-2B chain, Thiosulfate sulfurtransferase/rhodanese-like domain-containing protein 1, Mitogen-activated protein kinase 1, Alcohol dehydrogenase class-3, Low molecular weight phosphotyrosine protein phosphatase, Annexin A4, Septin-2, Glutathione S-transferase Mu 3, Protein BRICK1, Proteasome subunit beta type-2, Ubiquitin- conjugating enzyme E2 K, Protein S100-A1, Microtubule-associated protein 1A, Glutathione S-transferase Mu 1, Matrix metalloproteinase-24, Small integral membrane protein 22, Heparan-alpha-glucosaminide N-acetyltransferase, Specifically androgen-regulated gene protein, Abl interactor 1, Uncharacterized protein C6orf132, ADP-ribosylation factor 5, Isocitrate dehydrogenase [NADP] cytoplasmic, Glycolipid transfer protein, Tropomyosin alpha-4 chain and Natural resistance-associated macrophage protein 2;
wherein said sample comprises urinary exosomes and wherein said sample has been obtained from said subject;
wherein an increased level in said sample of one or more of said polypeptides selected from the group consisting of Transmembrane protein 256, Adipogenesis regulatory factor, Ragulator complex protein LAMTOR1, Plastin-2, Ras-related protein Rab-2A, Ras-related protein Rab-3B, Ras-related protein Rab-3D, Ras-related protein Rab-7a, V-type proton ATPase 16 kDa proteolipid subunit, Metalloreductase STEAP4, Protein DJ-1, Protein S100-P, Synaptotagmin-like protein 4, ADP-ribosylation factor-like protein 8B, Proton myo-inositol cotransporter, Ras-related protein Rab-6A, Tetraspanin-6, Claudin-10, Claudin-2, Claudin-3, GDP-mannose 4.6 dehydratase, Glucosamine-6-phosphate isomerase 1, Lysosome membrane protein 2, Major facilitator superfamily domain-containing protein 12, Melanophilin, Sepiapterin reductase, Thioredoxin domain-containing protein 17, 3-hydroxybutyrate dehydrogenase type 2, Calmodulin, Carboxypeptidase Q, Flotillin-2, Galectin-3-binding protein, P2X purinoceptor 4, Protein dopey-2, Serine/threonine-protein phosphatase 2A catalytic subunit alpha iso form, 2′-deoxynucleoside 5′-phosphate N-hydrolase 1, Acid ceramidase, Calbindin, CD81 antigen, Cytochrome b561, Enolase-phosphatase E1, Golgi phosphoprotein 3, Nicastrin, Probable serine carboxypeptidase CPVL, Ragulator complex protein LAMTORS, Ras-related protein Rab-27B, Secretory carrier-associated membrane protein 2, Spermine synthase, S-phase kinase-associated protein 1, Transmembrane 7 superfamily member 3, Tumor protein D52, Ubiquitin-conjugating enzyme E2 variant 2, UDP-glucose 6-dehydrogenase, Zinc-alpha-2-glycoprotein, Glycerophosphodiester phosphodiesterase domain-containing protein 3, EF-hand domain-containing protein D2, Ras-related protein Rab-14, Omega-amidase NIT2, Alpha-actinin-1, Monocarboxylate transporter 5, Ras-related protein Rab-12, Ras-related protein Rab-8A, Transmembrane protein 63A, V-type proton ATPase subunit d 1, Lipid phosphate phosphohydrolase 1, Integral membrane protein GPR155, E3 ubiquitin-protein ligase LRSAM1, HLA class II histocompatibility antigen. DM alpha chain, Ras-related protein Rab-9A, Aquaporin-7, Gamma-synuclein, Aspartate aminotransferase. cytoplasmic, Chloride intracellular channel protein 3, Destrin, GTPase HRas, Prostaglandin reductase 2, T-complex protein 1 subunit epsilon, Aldehyde dehydrogenase family 1 member A3, Annexin A3, Battenin, Cathepsin D, N(G).N(G)-dimethylarginine dimethylaminohydrolase 1, Neural proliferation differentiation and control protein 1, Proactivator polypeptide, Prostate-specific antigen, Protein lifeguard 3, Protein Niban, Protein spinster homolog 1, Ragulator complex protein LAMTOR2, Ragulator complex protein LAMTOR3, Synaptotagmin-7, Transmembrane protein 106B, Unconventional myosin-Vc, Vesicle-associated membrane protein 2, V-type proton ATPase subunit F, A-kinase anchor protein 7 isoforms alpha and beta, Arylsulfatase F, C-Jun-amino-terminal kinase-interacting protein 4, Deleted in malignant brain tumors 1 protein, Glia maturation factor beta, Glutamate carboxypeptidase 2, Glutathione synthetase, Hippocalcin-like protein 1, NAD(P)H-hydrate epimerase, Napsin-A, Phosphoacetylglucosamine mutase, Probable phospholipid-transporting ATPase IA, Quinone oxidoreductase, Retinol-binding protein 5, Serine incorporator 2, Solute carrier family 15 member 1, Sorcin, Sucrase-isomaltase. Intestinal, Actin-related protein ⅔ complex subunit 4, Actin-related protein ⅔ complex subunit 5, Adenine phosphoribosyltransferase, Costars family protein ABRACL, Ig alpha-1 chain C region, Interferon-induced transmembrane protein 3, Lactotransferrin, Purine nucleoside phosphorylase, Syntaxin-binding protein 4, Vacuolar protein-sorting-associated protein 25, Voltage-dependent anion-selective channel protein 1, CD9 antigen, Flotillin-1, Grancalcin, Mannose-1-phosphate guanyltransferase beta, Proteasome subunit alpha type-7, Ras-related protein Rab-18, Vacuolar protein sorting-associated protein 37C, Pancreatic secretory granule membrane major glycoprotein GP2, Lysosome-associated membrane glycoprotein 2, Ragulator complex protein LAMTOR4, Secretory carrier-associated membrane protein 1, Peptidyl-prolyl cis-trans isomerase FKBP1A, Transmembrane protein 176A, Thymosin beta-4, Haloacid dehalogenase-like hydrolase domain-containing protein 2, Cell division control protein 42 homolog, Ras-related protein Rab-17, Chloride intracellular channel protein 6, Choline transporter-like protein 4, Flavin reductase (NADPH), Ras-related protein Rab-10, Heme-binding protein 2, Fatty acid-binding protein. epidermal, Small integral membrane protein 5, Lysosomal-associated transmembrane protein 4A, Phytanoyl-CoA dioxygenase domain-containing protein 1, Proteasome subunit alpha type-5, Calmodulin-like protein 3, Presenilin-1, Ribosyldihydronicotinamide dehydrogenase [quinone], Translationally-controlled tumor protein, Lysosome-associated membrane glycoprotein 1, ADP-ribosyl cyclase 1, Myotrophin, Dynein light chain 2. cytoplasmic, Dihydropteridine reductase, Nicotinate-nucleotide pyrophosphorylase [carboxylating], Cell cycle control protein 50A, Eukaryotic translation initiation factor 4H, Protein tweety homolog 3, Sodium-dependent phosphate transport protein 2B, Ig lambda-2 chain C regions, Cellular retinoic acid-binding protein 2, Protein CutA, Proteasome subunit alpha type-4, Solute carrier family 35 member F6, Delta-aminolevulinic acid dehydratase, L-xylulose reductase, Uroplakin-1a, Comifin-A, Zinc finger protein 185, Transmembrane protein 8A, Prenylcysteine oxidase 1, Lysozyme C, Paralemmin-1, Carcinoembryonic antigen-related cell adhesion molecule 6, Sodium/glucose cotransporter 1, Prostaglandin reductase 1, Protein S100-A9, MICAL-like protein 1, Aquaporin-2, Glutathione S-transferase P, Ras-related protein Rab-8B, Transmembrane protease serine 2, Ras-related protein Rab-1B, Ras-related protein Rab-1A, Ras-related protein Rab-43, Synaptogyrin-2, HLA class II histocompatibility antigen. DRB1-15 beta chain, Lysosomal protein NCU-G1, CDC42 small effector protein 2, Ferritin heavy chain, Solute carrier family 35 member F2,Probable hydrolase PNKD, Cathepsin Z, Tubulin beta-2B chain, Mitogen-activated protein kinase 1, Alcohol dehydrogenase class-3, Low molecular weight phosphotyrosine protein phosphatase, Annexin A4, Septin-2, Glutathione S-transferase Mu 3, Protein BRICK1, Proteasome subunit beta type-2, Ubiquitin- conjugating enzyme E2 K, Protein S100-A1, Microtubule-associated protein 1A, Glutathione S-transferase Mu 1, Small integral membrane protein 22, Heparan-alpha-glucosaminide N-acetyltransferase, Specifically androgen-regulated gene protein, Abl interactor 1, Uncharacterized protein C6orf132, Isocitrate dehydrogenase [NADP] cytoplasmic, Glycolipid transfer protein, Tropomyosin alpha-4 chain and Natural resistance-associated macrophage protein 2 in comparison to a control level is indicative of prostate cancer in said subject; and/or
wherein a decreased level in said sample of one or more of said polypeptides selected from the group consisting of Protein S100-A6, Ras-related protein Rab-35, CD59 glycoprotein, Beta-2-microglobulin, 14-3-3 protein sigma, Src substrate cortactin, 14-3-3 protein theta, Inter-alpha-trypsin inhibitor heavy chain H4, Myristoylated alanine-rich C-kinase substrate, Tetraspanin-8, Collagen alpha-1(VI) chain, Signal transducing adapter molecule 1, Signal transducing adapter molecule 2, 1-phosphatidylinosito14.5-bisphosphate phosphodiesterase gamma-2, Cysteine-rich C-terminal protein 1, Plasma serine protease inhibitor, Tyrosine-protein kinase Lck, Phosphomevalonate kinase, Alpha/beta hydrolase domain-containing protein 14B, Probable almitoyltransferase ZDHHC1, Calcium-binding protein 39, Dynamin-2, Thiosulfate sulfurtransferase/rhodanese-like domain-containing protein 1, Matrix metalloproteinase-24 and ADP-ribosylation factor 5 in comparison to a control level is indicative of prostate cancer in said subject.

In one embodiment, the method comprises determining the level in a sample of one or more polypeptides selected from the group consisting of:
Transmembrane protein 256, Adipogenesis regulatory factor, Ragulator complex protein LAMTOR1, Plastin-2, Ras-related protein Rab-2A, Ras-related protein Rab-3B, Ras-related protein Rab-3D, Ras-related protein Rab-7a, V-type proton ATPase 16 kDa proteolipid subunit, Metalloreductase STEAP4, Protein DJ-1, Protein S100-P, Synaptotagmin-like protein 4, ADP-ribosylation factor-like protein 8B, Proton myo-inositol cotransporter, Ras-related protein Rab-6A, Tetraspanin-6, Claudin-10, Claudin-2, Claudin-3, GDP-mannose 4.6 dehydratase, Glucosamine-6-phosphate isomerase 1, Lysosome membrane protein 2, Major facilitator superfamily domain-containing protein 12, Melanophilin, Sepiapterin reductase, Thioredoxin domain-containing protein 17, 3-hydroxybutyrate dehydrogenase type 2, Calmodulin, Carboxypeptidase Q, Flotillin-2, Galectin-3-binding protein, P2X purinoceptor 4, Protein dopey-2, Protein S100-A6, Ras-related protein Rab-35, Serine/threonine-protein phosphatase 2A catalytic subunit alpha iso form, 2′-deoxynucleoside 5′-phosphate N-hydrolase 1, Acid ceramidase, Calbindin, CD59 glycoprotein, CD81 antigen, Cytochrome b561, Enolase-phosphatase E1, Golgi phosphoprotein 3, Nicastrin, Probable serine carboxypeptidase CPVL, Ragulator complex protein LAMTORS, Ras-related protein Rab-27B, Secretory carrier-associated membrane protein 2, Spermine synthase, S-phase kinase-associated protein 1, Transmembrane 7 superfamily member 3, Tumor protein D52, Ubiquitin-conjugating enzyme E2 variant 2, UDP-glucose 6-dehydrogenase, Zinc-alpha-2-glycoprotein and Glycerophosphodiester phosphodiesterase domain-containing protein 3.

In one embodiment, the method comprises determining the level in a sample of one or more polypeptides selected from the group consisting of:
Transmembrane protein 256, Adipogenesis regulatory factor, Ragulator complex protein LAMTOR1, Plastin-2, Ras-related protein Rab-2A, Ras-related protein Rab-3B, Ras-related protein Rab-3D, Ras-related protein Rab-7a, V-type proton ATPase 16 kDa proteolipid subunit, Metalloreductase STEAP4, Protein DJ-1, Protein S100-P, Synaptotagmin-like protein 4, ADP-ribosylation factor-like protein 8B, Proton myo-inositol cotransporter, Ras-related protein Rab-6A and Tetraspanin-6.

In one embodiment, the method comprises determining the level in a sample of one or more polypeptides selected from the group consisting of:
Transmembrane protein 256, Adipogenesis regulatory factor, Ragulator complex protein LAMTOR1, Plastin-2, Ras-related protein Rab-2A, Ras-related protein Rab-3B, Ras-related protein Rab-3D, Ras-related protein Rab-7a, V-type proton ATPase 16 kDa proteolipid subunit, Metalloreductase STEAP4, Protein DJ-1, Protein S100-P, Synaptotagmin-like protein 4, ADP-ribosylation factor-like protein 8B, Proton myo-inositol cotransporter, Ras-related protein Rab-6A, Tetraspanin-6, Claudin-3, Protein S100-A6 and UDP-glucose 6-dehydrogenase.

In one embodiment, the levels of the polypeptides described herein are determined by mass spectrometry.

In one embodiment, the levels of the polypeptides described herein are determined by an immunoassay, such as, but not limited to, Western blotting and ELISA.

In one embodiment, the method comprises determining the level in a sample of one or more polypeptides selected from the group consisting of: Transmembrane protein 256, Ragulator complex protein LAMTOR1, Ras-related protein Rab-3B, Flotillin-1, Flotillin-2 and Protein DJ-1. In one embodiment, the method comprises determining the level in a sample of one or more polypeptides selected from the group consisting of: Transmembrane protein 256, Ragulator complex protein LAMTOR1, Ras-related protein Rab-3B, Flotillin-1 and Flotillin-2. In some such embodiments the level in a sample is determined by Western blotting or another immunoassay based method, including ELISA.

In one embodiment, the method comprises determining the level in a sample of one or more polypeptides selected from the group consisting of:
Transmembrane protein 256, Adipogenesis regulatory factor, Ragulator complex protein LAMTOR1, Vesicle-associated membrane protein 2, V-type proton ATPase 16 kDa proteolipid subunit, Acid ceramidase, Prenylcysteine oxidase 1, Sorcin, Grancalcin, Ras-related protein Rab-7a, Tetraspanin-6, 3-hydroxybutyrate dehydrogenase type 2, EF-hand domain-containing protein D2, Flotillin-2, Ras-related protein Rab-3D, Adenine phosphoribosyltransferase, Calmodulin, Protein DJ-1, Retinol-binding protein 5, Ubiquitin-conjugating enzyme E2 variant 2, S-phase kinase-associated protein 1, ADP-ribosylation factor-like protein 8B, Cytochrome b561, GDP-mannose 4.6 dehydratase, Matrix metalloproteinase-24, CD59 glycoprotein, Claudin-2, Glutathione synthetase, Costars family protein ABRACL, Low molecular weight phosphotyrosine protein phosphatase, A-kinase anchor protein 7 iso forms alpha and beta, Actin-related protein ⅔ complex subunit 4, 2′-deoxynucleoside 5′-phosphate N-hydrolase 1, Dihydropteridine reductase, Ras-related protein Rab-3B, Vacuolar protein-sorting-associated protein 25, Cathepsin D, L-xylulose reductase, Mannose-1-phosphate guanyltransferase beta, Napsin-A, Purine nucleoside phosphorylase, Prostaglandin reductase 1, Ras-related protein Rab-2A, Protein S100-P, Small integral membrane protein 22, Synaptotagmin-7, Tetraspanin-8, Annexin A4, CD81 antigen, Flotillin-1, Interferon-induced transmembrane protein 3, Nicastrin, Lipid phosphate phosphohydrolase 1, Ras-related protein Rab-18, Transmembrane protein 63A, Transmembrane protease serine 2 and UDP-glucose 6-dehydrogenase.
These are polypeptides (proteins) which have a combined sensitivity and specificity of at least 160% (as set forth in Table 6 herein).

In one embodiment, the method comprises determining the level in a sample of one or more polypeptides selected from the group consisting of:
Transmembrane protein 256, Adipogenesis regulatory factor, Ragulator complex protein LAMTOR1, Vesicle-associated membrane protein 2, V-type proton ATPase 16 kDa proteolipid subunit, Acid ceramidase, Prenylcysteine oxidase 1, Sorcin, Grancalcin, Ras-related protein Rab-7a and Tetraspanin-6. These are polypeptides (proteins) which have a combined sensitivity and specificity of at least 170% (as set forth in Table 6 herein).

In another embodiment, the method comprises determining the level in a sample of one or more polypeptides set forth in Table 6 as having a combined sensitivity and specificity of at least 175% or 180%.

In another embodiment, the method comprises determining the level in a sample of one or more polypeptides selected from the group consisting of:

Vesicle-associated membrane protein 2, Prenylcysteine oxidase 1, Sorcin and Grancalcin.

In another embodiment, the method comprises determining the level in a sample of one or more polypeptides selected from the group consisting of:

Transmembrane protein 256, Ragulator complex protein LAMTOR1, V-type proton ATPase 16 kDa proteolipid subunit, Synaptotagmin-like protein 4, Claudin-3, Protein S100-A6, UDP-glucose 6-dehydrogenase, Adipogenesis regulatory factor, Ras-related protein Rab-2A, Ras-related protein Rab-3B, Ras-related protein Rab-7a, Protein DJ-1, Tetraspanin-6, Ras-related protein Rab-3D, Protein S100-P, Proton myo-inositol cotransporter, Plastin-2, Metalloreductase STEAP4, ADP-ribosylation factor-like protein 8B, Ras-related protein Rab-6A, Vesicle-associated membrane protein 2, Prenylcysteine oxidase 1, Sorcin and Grancalcin.

In one embodiment, the method comprises determining the level in a sample of one or more polypeptides that are identified in Table 2 herein as having a “Validated iBAQ ratio PAT:CTR” of at least 1.75 (e.g. at least 2, at least 3, at least 4, at least 5, at least 6, at least 7, at least 8, at least 9, at least 10, at least 15, at least 20, or at least 40).

In one embodiment, the method comprises determining the level in a sample of one or more polypeptides that are identified in Table 2 herein as having a “Validated iBAQ ratio PAT:CTR” of at least 1 (or more than 1). In another embodiment, the method comprises determining the level in a sample of one or more polypeptides that are identified in Table 2 herein as having a “Validated iBAQ ratio PAT:CTR” of less than 1.

In one embodiment, the method comprises determining the level in a sample of one or more polypeptides that are referred to above as being indicative of prostate cancer when their level is increased.

In another embodiment, the method comprises determining the level in a sample of one or more polypeptides that are referred to above as being indicative of prostate cancer when their level is decreased.

In some embodiments, the method comprises determining the level in a sample of one or more polypeptides (proteins) that are identified in Table 2 or Table 3 herein as having a “sensitivity” of at least 40%, at least 45%, at least 50%, at least 55%, at least 60%, at least 65%, at least 70%, at least 75%, at least 80%, at least 85% or at least 90%. In one embodiment, the level of a polypeptide (protein) identified in Table 2 herein as having a “sensitivity” of more than 40% is determined. In one embodiment, the level of a polypeptide (protein) identified in Table 2 herein as having a “sensitivity” of more than 50% is determined. In a preferred embodiment, the level of a polypeptide (protein) identified in Table 2 herein as having a “sensitivity” of more than 60% is determined. In another preferred embodiment, the level of a polypeptide (protein) identified in Table 2 herein as having a “sensitivity” of more than 70% is determined. In another preferred embodiment, the level of a polypeptide (protein) identified in Table 2 herein as having a “sensitivity” of more than 80% is determined.

In another embodiment, the method comprises determining the level in a sample of one or more polypeptides (proteins) that are identified in Table 2 herein as being detected in the validation study (validation analysis).

In another embodiment, the method comprises determining the level in a sample of one or more polypeptides (proteins) that are identified in Table 2 herein as being significantly altered in the validation study.

The Example herein describes certain preferred biomarkers that meet the following four criteria (see Table 2): (1) detected in validation study, (2) significantly altered in validation study, (3) sensitivity of above 40% and (4) ratio PAT versus CTR above 1.75. In relation to criteria (4), it is biomarkers whose level is increased in prostate cancer patients (samples) versus control that can have a PAT (patient) versus CTR (control) ratio of above 1.75. Analogously, for biomarkers whose level is decreased in prostate cancer patients (samples) versus control, an analogous criteria (4) may be applied, in which there is at least 1.75 times less expression of the biomarker in PAT versus CTR. In certain embodiments, the determination of the level of one or more polypeptides which meet (pass) all four of these criteria and which have a sensitivity of at least 50%, at least 55%, at least 60%, at least 65%, at least 70%, at least 75%, or at least 80% is preferred. In some embodiments , the determination of the level of one or more polypeptides which meet (pass) all four of these criteria and which have a sensitivity of at least 60% (e.g. at least 65%) is preferred. In some embodiments, the determination of the level of one or more polypeptides which meet (pass) all four of these criteria and which have a sensitivity of at least 75% is preferred. In some embodiments , the determination of the level of one or more polypeptides which meet (pass) all four of these criteria and which have a sensitivity of at least 80% is preferred.

In a preferred embodiment, the method comprises determining the level of Transmembrane protein 256.

In one embodiment, the method comprises determining the level of Ragulator complex protein LAMTOR1.

In one embodiment, the method comprises determining the level of V-type proton ATPase 16 kDa proteolipid subunit.

In one embodiment, the method comprises determining the level of Synaptotagmin-like protein 4.

In one embodiment, the method comprises determining the level of Claudin-3.

In one embodiment, the method comprises determining the level of Protein S100-A6.

In one embodiment, the method comprises determining the level of UDP-glucose 6-dehydrogenase.

In one embodiment, the method comprises determining the level of Adipogenesis regulatory factor.

In one embodiment, the method comprises determining the level of Ras-related protein Rab-2A.

In one embodiment, the method comprises determining the level of Ras-related protein Rab-3B.

In one embodiment, the method comprises determining the level of Ras-related protein Rab-7a.

In one embodiment, the method comprises determining the level of Protein DJ-1.

In one embodiment, the method comprises determining the level of Tetraspanin-6.

In one embodiment, the method comprises determining the level of Ras-related protein Rab-3D.

In one embodiment, the method comprises determining the level of Protein S100-P.

In one embodiment, the method comprises determining the level of Proton myo-inositol cotransporter.

In one embodiment, the method comprises determining the level of Plastin-2.

In one embodiment, the method comprises determining the level of Metalloreductase STEAP4.

In one embodiment, the method comprises determining the level of ADP-ribosylation factor-like protein 8B.

In one embodiment, the method comprises determining the level of Ras-related protein Rab-6A.

In one embodiment, the method comprises determining the level of Vesicle-associated membrane protein 2.

In one embodiment, the method comprises determining the level of Prenylcysteine oxidase 1.

In one embodiment, the method comprises determining the level of Sorcin.

In one embodiment, the method comprises determining the level of Grancalcin.

In one embodiment, the method comprises determining the level of Flotillin-1.

In one embodiment, the method comprises determining the level of Flotillin-2.

In some embodiments, the level of a single polypeptide (protein) is determined. In other embodiments, the level of more than one of the polypeptides is determined (e.g. the level of two or more polypeptides, or three or more polypeptides, or four or more polypeptides is determined). By “more than one” is meant 2, 3, 4, 5, 6, 7, 8, 9, 10 etc. . . . 246 (including all integers between 2 and 246). A determination of the level of each and every possible combination of the polypeptides can be performed.

Thus, in some embodiments multi-marker methods are performed. Determining the level of multiple of the polypeptides (biomarker multiplexing) may improve screening (e.g. diagnostic) accuracy.

In a preferred embodiment, the level of two of the stated polypeptides is determined. In another preferred embodiment, the level of three of the stated polypeptides is determined. In yet another preferred embodiment, the level of four of the stated polypeptides is determined.

In some embodiments, the level of a polypeptide selected from the group consisting of Transmembrane protein 256, Adipogenesis regulatory factor, Ragulator complex protein LAMTOR1, Ras-related protein Rab-2A, Ras-related protein Rab-3B, Ras-related protein Rab-3D, Ras-related protein Rab-7a, V-type proton ATPase 16 kDa proteolipid subunit, Metalloreductase STEAP4, Protein DJ-1, Protein S100-P, Synaptotagmin-like protein 4, Proton myo-inositol cotransporter and Tetraspanin-6 is determined in combination with determining the level of one of the other polypeptides set forth in Table 3 herein. Such a method is an example of a two-marker test. However, these combinations of two-markers can also be used in tests where a greater number of markers are determined.

In some embodiments, the level of a polypeptide selected from the group consisting of Plastin-2, ADP-ribosylation factor-like protein 8B and Ras-related protein Rab-6A is determined in combination with determining the level of two further of the stated polypeptides (e.g. two of the other polypeptides set forth in Table 3). Such a method is an example of a three-marker test. However, these combinations of three-markers can also be used in tests where a greater number of markers are determined.

Some preferred two-, three- and four-marker (polypeptide) combinations are described in Table 5 and are set out below:
Transmembrane protein 256 and Ragulator complex protein LAMTOR1
Transmembrane protein 256 and V-type proton ATPase 16 kDa proteolipid subunit
Transmembrane protein 256 and Synaptotagmin-like protein 4
Transmembrane protein 256 and Claudin-3
Transmembrane protein 256 and Protein S100-A6
Transmembrane protein 256 and UDP-glucose 6-dehydrogenase
Adipogenesis regulatory factor and Ragulator complex protein LAMTOR1
Ragulator complex protein LAMTOR1 and Ras-related protein Rab-2A
Ragulator complex protein LAMTOR1 and Ras-related protein Rab-3B
Ragulator complex protein LAMTOR1 and Ras-related protein Rab-7a
Ragulator complex protein LAMTOR1 and Protein DJ-1
Ragulator complex protein LAMTOR1 and Synaptotagmin-like protein 4
Ragulator complex protein LAMTOR1 and Tetraspanin-6
V-type proton ATPase 16 kDa proteolipid subunit and Ras-related protein Rab-3B
V-type proton ATPase 16 kDa proteolipid subunit and Ras-related protein Rab-3D
V-type proton ATPase 16 kDa proteolipid subunit and Protein DJ-1
V-type proton ATPase 16 kDa proteolipid subunit and Protein S100-P
Synaptotagmin-like protein 4 and Proton myo-inositol cotransporter
Ragulator complex protein LAMTOR1 and Plastin-2 and Metalloreductase STEAP4
Ragulator complex protein LAMTOR1 and ADP-ribosylation factor-like protein 8B and Ras-related protein Rab-6A
Ragulator complex protein LAMTOR1 and Metalloreductase STEAP4 and Protein S100-P
Transmembrane protein 256 and Adipogenesis regulatory factor and
Ragulator complex protein LAMTOR1 and V-type proton ATPase 16 kDa proteolipid subunit

However, these combinations of two-, three- and four-markers can also be used in tests where a greater number of markers are determined.

Thus, in preferred methods of the present invention, the level of both of the polypeptides set forth above in the specific two marker combinations is determined. In other preferred methods the level of all three of the polypeptides set forth above in the specific three- marker combinations is determined. In another preferred method, the level of all four of the polypeptides set forth above in the specific four- marker combination is determined.

In another embodiment, the method comprises determining the level of Transmembrane protein 256 in combination with (i.e. and) determining the level of at least one (e.g. 1, 2 or 3) of the other polypeptides (proteins) set forth in Tables 1, 2 or 3 herein. In a particular embodiment, the method comprises determining the level of Transmembrane protein 256 in combination with (i.e. and) determining the level of at least one (e.g. 1, 2 or 3) of the other polypeptides (proteins) identified in Table 2 (or Table 3) herein as having a “sensitivity” of more than 60%.

Other appropriate combinations of markers can be derived from Table 3 by combining two or more of the markers in Table 3 (e.g. 2, 3, 4, 5 or 6 markers, preferably 2, 3 or 4 markers) that results in one or more of the patients (P) (preferably the majority of the patients, e.g. 9, 10, 11, 12, 13, 14, 15 or 16 of the patients, more preferably all of the patients) being associated with a positive call (as indicated by a “1” in Table 3) from at least one marker in the combination. Put another way, other appropriate combinations of markers (sequences/polypeptides) can be derived from Table 3 by combining two or more of the sequence rows (e.g. 2, 3, 4, 5 or 6 sequence rows, preferably 2, 3 or 4 sequence rows) such that the combination of said two or more sequence rows has at least one positive call (as indicated by “1”) in one or more patient columns (P) (preferably the majority of the patient columns, e.g. 9, 10, 11, 12, 13, 14, 15 or 16 of the patient columns, more preferably all of the patient columns). By way of example, sequences (markers) 12, 13 and 14 would be an appropriate three-marker combination as when sequence rows 12, 13 and 14 are combined there is at least one positive call (“1”) in all of the patient columns (P).

In some embodiments, the method comprises determining the level of one or more of the polypeptides (proteins) as set forth in Table 6 herein (e.g. Prenylcysteine oxidase 1) in combination with (“and”) determining the level of one or more of the other polypeptides mentioned herein (for example in combination with determining the level of one or more of Vesicle-associated membrane protein 2, Prenylcysteine oxidase 1, Sorcin or Grancalcin, or for example in combination with determining the level of one or more of the other polypeptides in Table 3, or for example in combination with determining the level of one or more of the other polypeptides in Table 6).

In some embodiments, the method comprises determining the level of one or more (1, 2, 3, 4, 5 or 6) of the polypeptides selected from the group consisting of: Transmembrane protein 256, Ragulator complex protein LAMTOR1, Ras-related protein Rab-3B, Flotillin-1, Flotillin-2 and Protein DJ-1 in combination with (“and”) determining the level of one or more of the other polypeptides mentioned herein (for example in combination with determining the level of one or more of the other polypeptides in Table 3, or for example in combination with determining the level of one or more of the other polypeptides in Table 6).

In some embodiments, the method comprises determining the level of one or more (1, 2, 3, 4 or 5) of the polypeptides selected from the group consisting of: Transmembrane protein 256, Ragulator complex protein LAMTOR1, Ras-related protein Rab-3B, Flotillin-1 and Flotillin-2 in combination with (“and”) determining the level of one or more of the other polypeptides mentioned herein (for example in combination with determining the level of one or more of the other polypeptides in Table 3, or for example in combination with determining the level of one or more of the other polypeptides in Table 6).

In some embodiments of the present invention the level of one or more (or all) of the following polypeptides (proteins) is not determined: 14-3-3 protein sigma, 14-3-3 protein theta, Actin-related protein ⅔ complex subunit 4, Actin-related protein ⅔ complex subunit 5, ADP-ribosylation factor-like protein 8B, Annexin A3, Beta-2-microglobulin, Calcium-binding protein 39, Calmodulin, CD81 antigen, CD9 antigen, Claudin-3, Destrin, Ferritin heavy chain, Flotillin-1, Myristoylated alanine-rich C-kinase substrate, Plastin-2, Protein DJ-1, Ras-related protein Rab-10, Ras-related protein Rab-12, Ras-related protein Rab-14, Ras-related protein Rab-1A, Ras-related protein Rab-1B, Ras-related protein Rab-7a, Ras-related protein Rab-8A, Ras-related protein Rab-8B, Septin-2, Translationally-controlled tumor protein, Vesicle-associated membrane protein 2.

In some embodiments of the present invention the level of one or more (or all) of the following polypeptides (proteins) is not determined: ADP-ribosylation factor-like protein 8B, Calmodulin, CD81 antigen, Claudin-3, Plastin-2, Protein DJ-1, Ras-related protein Rab-7a.

In some embodiments of the present invention the level of one or more (or all) of the following polypeptides (proteins) is not determined: Septin-2, CD81 antigen, Myristoylated alanine-rich C-kinase substrate, Ras-related protein Rab-14, Peptidyl-prolyl cis-trans isomerase FKBP1A.

In some embodiments of the present invention the level of transmembrane protease serine 2 is not determined.

In some embodiments of the present invention the level of prostate-specific antigen is not determined.

In some embodiments of the present invention the level of one or more (or all) of the following polypeptides (proteins) is not determined: Adipogenesis regulatory factor, Plastin-2, Ras-related protein Rab-2A, Ras-related protein Rab-3B, Ras-related protein Rab-3D, Metalloreductase STEAP4, Protein DJ-1, Protein S100-P, GDP-mannose 4.6 dehydratase, Lysosome membrane protein 2, 3-hydroxybutyrate dehydrogenase type 2, Protein S100-A6, 2′-deoxynucleoside 5′-phosphate N-hydrolase 1, Acid ceramidase, CD59 glycoprotein, CD81 antigen, Ragulator complex protein LAMTORS, Spermine synthase, Tumor protein D52, Zinc-alpha-2-glycoprotein, Alpha-actinin-1, Beta-2-microglobulin, Lipid phosphate phosphohydro lase 1, 14-3-3 protein sigma, Gamma-synuclein, Inter-alpha-trypsin inhibitor heavy chain H4, Aldehyde dehydrogenase family 1 member A3, Annexin A3, Cathepsin D, N(G).N(G)-dimethylarginine dimethylaminohydrolase 1, Proactivator polypeptide, Prostate-specific antigen, Protein Niban, Glutamate carboxypeptidase 2, Glutathione synthetase, Myristoylated alanine-rich C-kinase substrate, NAD(P)H-hydrate epimerase, Phosphoacetylglucosamine mutase, Sorcin, Adenine phosphoribosyltransferase, Costars family protein ABRACL, Lactotransferrin, Purine nucleoside phosphorylase, Voltage-dependent anion-selective channel protein 1, Collagen alpha-1(VI) chain, CD9 antigen, Flotillin-1, Mannose-1-phosphate guanyltransferase beta, Proteasome subunit alpha type-7, Pancreatic secretory granule membrane major glycoprotein GP2, Peptidyl-prolyl cis-trans isomerase FKBP1A, Flavin reductase (NADPH), Ras-related protein Rab-10, Heme-binding protein 2, Fatty acid-binding protein. epidermal, Proteasome subunit alpha type-5, Eukaryotic translation initiation factor 4H, Cellular retinoic acid-binding protein 2, L-xylulose reductase, Protein S100-A9, Alpha/beta hydrolase domain-containing protein 14B, Glutathione S-transferase P, Transmembrane protease serine 2, Ferritin heavy chain, Cathepsin Z, Annexin A4, Septin-2, Glutathione S-transferase Mu 3, Proteasome subunit beta type-2, Glutathione S-transferase Mu 1, Specifically androgen-regulated gene protein, ADP-ribosylation factor 5, Isocitrate dehydrogenase [NADP] cytoplasmic.

Exemplary amino acid sequences of the above named polypeptides are provided herein by reference to the corresponding Uniprot Accession Number (see e.g. Table 1 herein) (http://www.uniprot.org/).

As discussed above, the present invention provides a method for screening for prostate cancer in a subject. Alternatively viewed, the present invention provides a method of diagnosing prostate cancer in a subject. Alternatively viewed, the present invention provides a method for the prognosis of prostate cancer in a subject (prognosis of the future severity, course and/or outcome of prostate cancer). Alternatively viewed, the present invention provides a method of determining the clinical severity of prostate cancer in a subject. Alternatively viewed, the present invention provides a method for predicting the response of a subject to therapy. Alternatively viewed, the present invention provides a method for detecting the recurrence of prostate cancer. Alternatively viewed, the present invention provides a method of assessing qualification of a subject for a first (or follow-up) biopsy (prostate biopsy). Alternatively viewed, the present invention provides a method for determining the aggresiveness of prostate cancer, e.g. distinguishing between indolent and aggressive cancer (and thus may e.g. inform a decision between active surveillance and treatment). Alternatively viewed, the present invention provides a method of monitoring a subject (patient) under active surveillance.

Thus, the method of screening for prostate cancer in accordance with the present invention can be used, for example, for diagnosing prostate cancer, for the prognosis of prostate cancer, for monitoring the progression of prostate cancer, for determining the clinical severity of prostate cancer, for predicting the response of a subject to therapy, for determining the efficacy of a therapeutic regime being used to treat prostate cancer, for detecting the recurrence of prostate cancer, for assessing qualification of a subject for a first (or follow-up) biopsy (prostate biopsy), for distinguishing between indolent and aggressive cancer, or for monitoring a subject (patient) under active surveillance.

Thus, in one aspect the present invention provides a method for diagnosing prostate cancer in a subject. In some embodiments, a positive diagnosis is made if the level of one or more of the polypeptides (proteins/biomarkers) in the sample is altered (increased or decreased as the case may be) in comparison to a control level. Polypeptides for which an increased level is indicative of (e.g. diagnostic of) prostate cancer are described herein. Polypeptides for which a decreased level is indicative of (e.g. diagnostic of) prostate cancer are described herein.

In another aspect, the present invention provides a method for selecting patients suspected of having prostate cancer for further diagnosis, such as a first or a follow-up biopsy procedure. In some embodiments, a positive indication is made if the level of one or more of the polypeptides (proteins/biomarkers) in the sample is altered (increased or decreased as the case may be) in comparison to a control level. Polypeptides for which an increased level is indicative of (e.g. diagnostic of) prostate cancer are described herein. Polypeptides for which a decreased level is indicative of (e.g. diagnostic of) prostate cancer are described herein.

In another aspect, the present invention provides a method for determining whether a patient is likely to have an indolent or aggressive form of prostate cancer. In some embodiments, the prostate cancer is designated as aggressive if the level of one or more of the polypeptides (proteins/biomarkers) in the sample is altered (increased or decreased as the case may be) in comparison to a control level.

In another aspect, the present invention provides a method for the prognosis of prostate cancer in a subject. In such methods the level of one or more of polypeptides (proteins/biomarkers) discussed above in the sample is indicative of the future severity, course and/or outcome of prostate cancer. For example, an alteration (increase or decrease as the case may be) in the level of one or more of the polypeptides (proteins/biomarkers) in the sample in comparison to a control level may indicate a poor prognosis. A highly altered level may indicate a particularly poor prognosis.

Thus, in some embodiments, an increased level of one or more of the polypeptides for which an increased level is indicative of prostate cancer is suggestive of (i.e. indicative of) a poor prognosis. In some embodiments, a decreased level of one or more of the polypeptides for which a decreased level is indicative of prostate cancer is suggestive of (i.e. indicative of) a poor prognosis. Conversely, if one or more polypeptides has an unaltered level (or an essentially unaltered level) that can be indicative of a good prognosis.

Serial (periodic) measuring of the level of one or more of the polypeptides (proteins/biomarkers) may also be used for prognostic purposes looking for either increasing or decreasing levels over time. In some embodiments, an altering level (increase or decrease) of one or more of the polypeptides over time (in comparison to a control level) may indicate a worsening prognosis. In some embodiments, an altering level (increase or decrease) of one or more of the polypeptides over time (in comparison to a control level) may indicate an improving prognosis. Thus, the methods of the present invention can be used to monitor disease progression. Such monitoring can take place before, during or after treatment of prostate cancer by surgery or therapy. Thus, in one aspect the present invention provides a method for monitoring the progression of prostate cancer in a subject.

Methods of the present invention can be used in the active monitoring of patients which have not been subjected to surgery or therapy, e.g. to monitor the progress of prostate cancer in untreated patients. Again, serial measurements can allow an assessment of whether or not, or the extent to which, the prostate cancer is worsening, thus, for example, allowing a more reasoned decision to be made as to whether therapeutic intervention is necessary or advisable.

Monitoring can also be carried out, for example, in an individual who is thought to be at risk of developing prostate cancer, in order to obtain an early, and ideally pre-clinical, indication of prostate cancer.

In another aspect, the present invention provides a method for determining the clinical severity of prostate cancer in a subject. In such methods the level of one or more of the polypeptides (proteins/biomarkers) in the sample shows an association with the severity of the prostate cancer. Thus, the level of one or more of polypeptides is indicative of the severity of the prostate cancer In some embodiments, the more altered (more increased or more decreased as the case may be) the level of one or more of the polypeptides in comparison to a control level, the greater the likelihood of a more severe form of prostate cancer. In some embodiments the methods of the invention can thus be used in the selection of patients for therapy.

Serial (periodical) measuring of the level of one or more of the polypeptides (proteins/biomarkers) may also be used to monitor the severity of prostate cancer looking for either increasing or decreasing levels over time. Observation of altered levels (increase or decrease as the case may be) may also be used to guide and monitor therapy, both in the setting of subclinical disease, i.e. in the situation of “watchful waiting” (also known as “active surveillance”) before treatment or surgery, e.g. before initiation of pharmaceutical therapy, or during or after treatment to evaluate the effect of treatment and look for signs of therapy failure.

The present invention also provides a method for predicting the response of a subject to therapy. In such methods the choice of therapy may be guided by knowledge of the level of one or more of the polypeptides in the sample.

The present invention also provides a method of determining (or monitoring) the efficacy of a therapeutic regime being used to treat prostate cancer. In such methods, an alteration (increase or decrease as the case may be) in the level of one or more of the polypeptides indicates the efficacy of the therapeutic regime being used. For example, if the level of one or more of the polypeptides for which an increased level is indicative of prostate cancer is reduced during (or after) therapy, this is indicative of an effective therapeutic regime. Conversely, for example, if the level of one or more of the polypeptides for which a decreased level is indicative of prostate cancer is increased during (or after) therapy, this is indicative of an effective therapeutic regime. In such methods, serial (periodical) measuring of the level of one or more of the polypeptides (proteins/biomarkers) over time can also be used to determine the efficacy of a therapeutic regime being used.

The present invention also provides a method for detecting the recurrence of prostate cancer.

The features and discussion herein in relation to the method of screening for prostate cancer (e.g. in relation to preferred polypeptides or combinations thereof discussed above) apply, mutatis mutandis, to the other related methods of present invention (e.g. to a method of diagnosing prostate cancer).

In one embodiment, the invention provides the use of the methods (e.g. screening, diagnostic or prognostic methods) in conjunction other known screening, diagnostic or prognostic methods (e.g. the PSA test). Thus, for example, the methods of the invention can be used to confirm a diagnosis of prostate cancer in a subject. In some embodiments the methods of the present invention are used alone.

A yet further aspect provides a kit for the screening (e.g. diagnosis or prognosis) of prostate cancer which comprises an agent suitable for determining the level of one or more of the polypeptides (proteins/biomarkers) described above, or fragments thereof, in a sample. Preferred agents are antibodies. In preferred aspects said kits are for use in the methods of the invention as described herein. Preferably, said kits comprise instructions for use of the kit components, for example in diagnosis. In some embodiments, the kit is a multimarker kit. Thus, in some embodiments the kit comprises more than one agent (e.g. two, three or four distinct agents), each agent being suitable for determining the level of one of the polypeptides (proteins/biomarkers) described above, or fragments thereof, in a sample. Using such kits (multimarker kits) the level of multiple (e.g. two, three or four) polypeptides may be determined. Exemplary groups (combinations) of polypeptides (markers) whose level may be determined using such multimarker kits are discussed elsewhere herein in relation to other aspects of the invention. In a preferred embodiment of such multimarker kits, the agent suitable for determining the level of a polypeptide is an antibody.

The level of the polypeptide (protein) in question can be determined by analysing the sample which has been obtained from or removed from the subject by an appropriate means. The determination is typically carried out in vitro.

Levels of one or more of the polypeptides in the sample can be measured (determined) by any appropriate assay, a number of which are well known and documented in the art and some of which are commercially available. The level of one or more of the polypeptides (proteins/biomarkers) can be determined e.g. by an immunoassay such as a radioimmunoassay (RIA) or fluorescence immunoassay, immunoprecipitation and immunoblotting (e.g. Western blotting) or Enzyme-Linked ImmunoSorbent Assay (ELISA). Immunoassays are a preferred technique for determining the levels of one or more of the polypeptides in accordance with the present invention.

Preferred assays are ELISA-based assays, although RIA-based assays can also be used effectively. Both ELISA- and RIA-based methods can be carried out by methods which are standard in the art and would be well known to a skilled person. Such methods generally involve the use of an antibody to a relevant polypeptide under investigation, or fragment thereof, which is incubated with the sample to allow detection of said polypeptide (or fragment thereof) in the sample. Any appropriate antibodies can be used and examples of these are described in the prior art. For example, an appropriate antibody to a polypeptide under investigation, or an antibody which recognises particular epitopes of said polypeptide, can be prepared by standard techniques, e.g. by immunization of experimental animals, which are know to a person skilled in the art. The same antibody to a given polypeptide under investigation or fragments thereof can generally be used to detect said polypeptide in either a RIA-based assay or an ELISA-based assay, with the appropriate modifications made to the antibody in terms of labelling etc., e.g. in an ELISA assay the antibodies would generally be linked to an enzyme to enable detection. Any appropriate form of assay can be used, for example the assay may be a sandwich type assay or a competitive assay.

In simple terms, in ELISA an unknown amount of antigen is affixed to a surface, and then a specific antibody is washed over the surface so that it can bind to the antigen. This antibody is linked to an enzyme, and in the final step a substance is added that the enzyme can convert to some detectable signal. Thus in the case of fluorescence ELISA, when light of the appropriate wavelength is shone upon the sample, any antigen/antibody complexes will fluoresce so that the amount of antigen in the sample can be determined through the magnitude of the fluorescence. For RIA, a known quantity of an antigen is made radioactive, frequently by labeling it with gamma-radioactive isotopes of iodine attached to tyrosine. This radiolabeled antigen is then mixed with a known amount of antibody for that antigen, and as a result, the two chemically bind to one another. Then, a sample from a patient containing an unknown quantity of that same antigen is added. This causes the unlabeled (or “cold”) antigen from the sample to compete with the radiolabeled antigen for antibody binding sites. As the concentration of “cold” antigen is increased, more of it binds to the antibody, displacing the radiolabeled variant, and reducing the ratio of antibody-bound radiolabeled antigen to free radiolabeled antigen. The bound antigens are then separated from the unbound ones, and the radioactivity of the free antigen remaining in the supernatant is measured. A binding curve can then be plotted, and the exact amount of antigen in the patient's sample can be determined. Measurements are usually also carried out on standard samples with known concentrations of marker (antigen) for comparison.

In some embodiments, the level of Flotillin-2 is determined by an ELISA-based assay.

In some embodiments, the level of Protein DJ-1 is determined by an ELISA-based assay.

In some embodiments, immunohistochemistry with appropriate antibodies could be carried out.

The use of immunoblotting (e.g. Western blotting) can also be used for measuring the level of one or more of the polypeptides in accordance with the present invention.

Preferred agents for use in determining the level of one or more of the polypeptides in accordance with the present invention are antibodies (antibodies to the polypeptide whose level is to be determined).

In other preferred embodiments, the level of one or more of the polypeptides in the sample can be measured (determined) by mass spectrometry. Suitable mass spectrometry methods (and associated data processing techniques) are well known and documented in the art. A particularly preferred mass spectrometry method (and associated data processing techniques) for determining the level of one or more of the polypeptides in the sample is described herein in the Example. In some embodiments mass spectrometry (and associated data processing techniques) is used to obtain a ratio of the level of a polypeptide in the sample in comparison to a control.

In accordance with the present invention, a quantitative, semi-quantitative or qualitative assessment (determination) of the level of one or more of the polypeptides can be made.

It is well understood in the art that when detecting the presence of a protein in a sample, it is not necessary to detect the presence of the full-length protein (i.e. the entire protein sequence); detecting the presence of a fragment of a protein can be indicative of the presence of the entire protein.

Thus, in certain embodiments of the methods of the invention described herein, any fragments of the polypeptides, in particular naturally occurring fragments, can be analysed as an alternative to the polypeptides themselves (full length polypeptides). Suitable fragments for analysis should be characteristic of the full-length protein. Suitable fragments can be at least 6 consecutive amino acids in length. For example, at least 7, at least 8, at least 9, at least 10, at least 15, at least 20, at least 25, at least 50, at least 75, at least 100, at least 150, at least 200 or at least 500 consecutive amino acids in length. Suitable fragments can represent at least 5%, at least 10%, at least 20%, at least 30%, at least 40%, at least 50%, at least 60%, at least 70%, at least 80%, or at least 90% of the length of the full-length polypeptide (protein).

In some embodiments the level of the full-length polypeptide is determined.

Reference herein to the “polypeptides” whose level is to be determined in accordance with the invention includes reference to all forms of said polypeptides (as appropriate) which might be present in a subject, including derivatives, mutants and analogs thereof, in particular fragments thereof or modified forms of the polypeptides or their fragments. Exemplary and preferred modified forms include forms of these molecules which have been subjected to post translational modifications such as glycosylation or phosphorylation. In some embodiments, the level of unmodified forms of the polypeptides (or their fragments) is determined.

The “increase” in the level or “increased” level of one or more of the polypeptides as described herein includes any measurable increase or elevation of the polypeptide (protein/biomarker) in question when the polypeptide in question is compared with a control level. Preferably, the level is significantly increased, compared to the level found in an appropriate control sample or subject. More preferably, the significantly increased levels are statistically significant, preferably with a probability value of <0.05. Viewed alternatively, an increase in level of the polypeptide of ≧2%, ≧3%, ≧5%, ≧10%, ≧25%, ≧50%, ≧75%, ≧100%, ≧200%, ≧300%, ≧400%, ≧500%, ≧600%, ≧700%, ≧800%, ≧900%, ≧1000%, ≧2000%, ≧5000%, or ≧10,000% compared to the level found in an appropriate control sample or subject (i.e. when compared to a control level) is indicative of the presence of prostate cancer. In a preferred embodiment the increase is ≧75% compared to the level found in an appropriate control sample or subject.

In some embodiments, the increase in level (e.g. of Transmembrane protein 256, Ragulator complex protein LAMTOR1, Ras-related protein Rab-3B, Flotillin-1 or Flotillin-2) is ≧50%, ≧75%, ≧100%, ≧150%, ≧200%, ≧250% or ≧500% compared to the level found in an appropriate control sample or subject, for example as determined by Western blotting.

In some embodiments, the increase in level (e.g. of Flotillin-2 or Protein DJ-1) is ≧50%, ≧75%, ≧100%, ≧150%, ≧200%, ≧250% or ≧500% compared to the level found in an appropriate control sample or subject, for example as determined by an ELISA-based assay.

In some embodiments, for those polypeptides described herein whose level is increased in prostate cancer samples in comparison to a control level, a level (concentration) of at least 10 pg polypeptide/μg (total) exosomal protein, at least 25 pg polypeptide/μg (total) exosomal protein, at least 50 pg polypeptide/μg (total) exosomal protein, at least 100 pg polypeptide/μg (total) exosomal protein, at least 200 pg polypeptide/μg (total) exosomal protein, at least 300 pg polypeptide/μg (total) exosomal protein, at least 400 pg polypeptide/μg (total) exosomal protein, at least 0.5 ng polypeptide/μg (total) exosomal protein, at least 0.75 ng polypeptide/μg (total) exosomal protein, at least 1 ng polypeptide/μg (total) exosomal protein, at least 1.5 ng polypeptide/μg (total) exosomal protein, at least 2 ng polypeptide/μg (total) exosomal protein, at least 3 ng polypeptide/μg (total) exosomal protein, at least 4 ng polypeptide/μg (total) exosomal protein, at least 5 ng polypeptide/μg (total) exosomal protein, at least 10 ng polypeptide/μg (total) exosomal protein, at least 25 ng polypeptide/μg (total) exosomal protein, at least 50 ng polypeptide/μg (total) exosomal protein or at least 100 ng polypeptide/μg (total) exosomal protein in a sample is indicative of prostate cancer in a subject. In some embodiments, concentrations may be determined by an ELISA-based assay.

The “decrease” in the level or “decreased” level of one or more of the polypeptides as described herein includes any measurable decrease or reduction of the polypeptide (protein/biomarker) in question when the polypeptide in question is compared with a control level. Preferably, the level is significantly decreased, compared to the level found in an appropriate control sample or subject. More preferably, the significantly decreased levels are statistically significant, preferably with a probability value of <0.05. Viewed alternatively, a decrease in level of the polypeptide of ≧2%, ≧3%, ≧5%, ≧10%, ≧25%, ≧50%, ≧75%, ≧100%, ≧200%, ≧300%, ≧400%, ≧500%, ≧600%, ≧700%, ≧800%, ≧900%, ≧1000%, ≧2000%, ≧5000%, or ≧10,000% compared to the level found in an appropriate control sample or subject (i.e. when compared to a control level) is indicative of the presence of prostate cancer. In a preferred embodiment the decrease is ≧50% compared to the level found in an appropriate control sample or subject.

In some embodiments, for those polypeptides described herein whose level is decreased in prostate cancer samples in comparison to a control level, a level (concentration) of less than 10 pg polypeptide/μg (total) exosomal protein, less than 25 pg polypeptide/μg (total) exosomal protein, less than 50 pg polypeptide/μg (total) exosomal protein, less than 100 pg polypeptide/μg (total) exosomal protein, less than 200 pg polypeptide/μg (total) exosomal protein, less than 300 pg polypeptide/μg (total) exosomal protein, less than 400 pg polypeptide/μg (total) exosomal protein, less than 0.5 ng polypeptide/μg (total) exosomal protein, less than 0.75 ng polypeptide/μg (total) exosomal protein, less than 1 ng polypeptide/μg (total) exosomal protein, less than 1.5 ng polypeptide/μg (total) exosomal protein, less than 2 ng polypeptide/μg (total) exosomal protein, less than 3 ng polypeptide/μg (total) exosomal protein, less than 4 ng polypeptide/μg (total) exosomal protein, less than 5 ng polypeptide/μg (total) exosomal protein, less than 10 ng polypeptide/μg (total) exosomal protein, less than 25 ng polypeptide/μg (total) exosomal protein, less than 50 ng polypeptide/μg (total) exosomal protein or less than 100 ng polypeptide/μg (total) exosomal protein in a sample is indicative of prostate cancer in a subject. In some embodiments, concentrations may be determined by an ELISA-based assay.

A “control level” is the level of a polypeptide in a control subject (e.g. in a sample that has been obtained from a control subject). Appropriate control subjects or samples for use in the methods of the invention would be readily identified by a person skilled in the art. Such subjects might also be referred to as “normal” subjects or as a reference population. Examples of appropriate control subjects would include healthy subjects, for example, individuals who have no history of any form of prostate disease (e.g. prostate cancer) and no other concurrent disease, or subjects who are not suffering from, and preferably have no history of suffering from, any form of prostate disease, in particular individuals who are not suffering from, and preferably have no history of suffering from, prostate cancer. Preferably control subjects are not regular users of any medication. In a preferred embodiment control subjects are healthy subjects.

The control level may correspond to the level of the equivalent polypeptide in appropriate control subjects or samples, e.g. may correspond to a cut-off level or range found in a control or reference population. Alternatively, said control level may correspond to the level of the marker (polypeptide) in question in the same individual subject, or a sample from said subject, measured at an earlier time point (e.g. comparison with a “baseline” level in that subject). This type of control level (i.e. a control level from an individual subject) is particularly useful for embodiments of the invention where serial or periodic measurements of polypeptide levels in individuals, either healthy or ill, are taken looking for changes in the levels of the polypeptide(s). In this regard, an appropriate control level will be the individual's own baseline, stable, nil, previous or dry value (as appropriate) as opposed to a control or cutoff level found in the general population. Control levels may also be referred to as “normal” levels or “reference” levels. The control level may be a discrete figure or a range.

Although the control level for comparison could be derived by testing an appropriate set of control subjects, the methods of the invention would not necessarily involve carrying out active tests on control subjects as part of the methods of the present invention but would generally involve a comparison with a control level which had been determined previously from control subjects and was known to the person carrying out the methods of the invention.

The sample which is tested according to the methods of the invention is a sample comprising urinary exosomes. In accordance with the present invention urinary exosomes can comprise (contain), or be suspected of comprising (containing), the polypeptide(s) (exosomal polypeptides/ exosomal proteins) whose level is to be determined. In other words, the methods of the invention involve the determination of levels of one or more polypeptides that are present in urinary exosomes (exosomes present in the urine). Exosomes are typically 30-150nm vesicles released by cells. Typically the sample has been obtained from (removed from) a subject, preferably a human male subject. In other aspects, the method further comprises a step of obtaining a sample from the subject.

In some embodiments the sample is a urine sample. In some embodiments the sample is derived from urine. Urine (and samples derived from urine e.g. isolated or partially isolated urinary exosomes) represents an attractive type of sample because it is easy to obtain (non-invasively) and its composition can reflect changes in the functioning of the prostate and other organs of the urogenital tract. In addition, the composition of urine is less complex than the composition of some other sample types, e.g. blood. In some embodiments the urine sample is used (processed) within 2 hours of having being collected from the subject. In some embodiments the urine sample is collected in the morning. In some embodiments, the urine sample may be a urine sample that has been collected without performing prostatic massage prior to urine collection. In some embodiments, the sample may be a sample derived from urine (e.g. isolated or partially isolated urinary exosomes), wherein said urine has been collected without performing prostatic massage prior to urine collection.

The term “sample” also encompasses any material derived by processing a biological sample (e.g. derived by processing a urine sample). Derived materials include isolated (or substantially or partially isolated) urinary exosomes from the sample. Processing of biological samples to obtain a test sample may involve one or more of: filtration, distillation, centrifugation, extraction, concentration, dilution, purification, inactivation of interfering components, addition of reagents, and the like. In some methods of the present invention, a sample comprising urinary exosomes (e.g. a urine sample) is subjected to a processing step, e.g. to isolate or partially isolate urinary exosomes, e.g. as described elsewhere herein.

In a preferred embodiment the sample comprises (or consists of or consists essentially of) isolated urinary exosomes. By isolated urinary exosomes is meant that the urinary exosomes are free from (or substantially free from) other urine components. Thus, in a preferred embodiment the sample is an isolated (or purified) sample of urinary exosomes. Isolated (e.g. purified) urinary exosomes can be resuspended in (or mixed with) an appropriate buffer (e.g. PBS) prior to analysis. Samples can contain urinary exosomes (e.g. isolated or purified urinary exosomes) and other non-urine components.

Any suitable method for isolating urinary exosomes may be employed. Urinary exosomes may be isolated from urine by serial centrifugation. A suitable method for isolating urinary exosomes by serial centrifugation is described herein in the Example. In this exemplary method, urine is centrifuged at 2,000 g for 15 min, and then at 10,000 g for 30 min discarding the pellet at each step. The exosomes present in the supernatant are then pelleted at 100,000 g for 70 min and washed with PBS. Exosomes are then resuspended again in PBS, filtrated through a 200 nm pore filter and pelleted at 100,000 g for 70 min. The pellet is resuspended in 50-100 μ1PBS and stored at -80 ° C. Thus, urinary exosomes for use in the methods of the present invention are capable of being isolated by such a serial centrifugation method.

Another suitable method for isolating urinary exosomes is to use antibody capture with an antibody that specifically binds to exosomal membrane proteins. Moreover, to specifically isolate urinary exosomes that originate from prostate cells, an antibody against a prostate-specific protein could be used. Antibodies can be bound to a bead or particle that facilitates isolation of urinary exosomes.

Commercially available kits may be used for the isolation of exosomes. Such kits include, but are not limited to, kits from Life Technologies (Catalogue number #4484452), Exiqon (Catalogue number #300102), Norgen Biotek Corp (Catalogue number #47200), System Biosciences (Catalogue number #EXOTC 50A-1), Cell Guidance Systems (Catalogue number #EX01) and 101 Bio (Catalogue number #P120).

In some embodiments, urinary exosomes are enzymatically (e.g. trypsin) digested (e.g. in solution digestion) prior to analysis of the levels of polypeptides therein. Such enzymatic digestion of urinary exosomes is typically performed when the level of one or more of the polypeptides therein is to be determined using mass spectrometry. An appropriate protocol for the enzymatic digestion of urinary exosomes prior to mass spectrometry analysis is provided herein in the Example.

In some embodiments, the urinary exosomes are disrupted (e.g. denatured) prior to determination of the level of one or more of the polypeptides therein.

Samples can be used immediately or can be stored for later use (e.g. at −80° C.).

In some embodiments, relatively low amounts of urinary exosomes are required in order to detect (e.g. by Western blot) a polypeptide whose level is to be determined. Thus, in some embodiments, the sample may comprise less than 10 μg exosomal protein, less than 5 μg exosomal protein, less than 2 μg exosomal protein, less than 1 iug exosomal protein, less than 0.5 μg exosomal protein, less than 0.25 μg exosomal protein, less than 100 ng exosomal protein, less than 50 ng exosomal protein or less than 25 ng exosomal protein. In some embodiments, the sample may comprise at least 25 ng exosomal protein, at least 50 ng exosomal protein, at least 100 ng exosomal protein, at least 0.25 μg exosomal protein, at least 0.5 μg exosomal protein, at least 1 μg exosomal protein, at least 2 μg exosomal protein, at least 5 μg exosomal protein or at least 10 μg exosomal protein. Exosomal protein may be total exosomal protein.

The methods of the invention as described herein can be carried out on any type of subject which is capable of suffering from prostate cancer. The methods are generally carried out on mammals, for example humans, primates (e.g. monkeys), laboratory mammals (e.g. mice, rats, rabbits, guinea pigs), livestock mammals (e.g. horses, cattle, sheep, pigs) or domestic pets (e.g. cats, dogs). Preferably the subject is a human.

In one embodiment, the subject (e.g. a human) is a subject at risk of developing prostate cancer or at risk of the occurrence of prostate cancer (e.g. a healthy subject or a subject not displaying any symptoms of prostate cancer or any other appropriate “at risk” subject). In another embodiment the subject is a subject having, or suspected of having (or developing), prostate cancer.

In some aspects, a method of the invention may further comprise an initial step of selecting a subject (e.g. a human subject) at risk of developing prostate cancer or having, or suspected of having (or developing), prostate cancer. The subsequent method steps can be performed on a sample from such a selected subject.

In another aspect, the present invention provides method of screening for prostate cancer in a subject, said method comprising

determining the level in a sample of one or more polypeptides selected from the group consisting of:
Transmembrane protein 256, Adipogenesis regulatory factor, Ragulator complex protein LAMTOR1, Plastin-2, Ras-related protein Rab-2A, Ras-related protein Rab-3B, Ras-related protein Rab-3D, Ras-related protein Rab-7a, V-type proton ATPase 16 kDa proteolipid subunit, Metalloreductase STEAP4, Protein DJ-1, Protein S100-P, Synaptotagmin-like protein 4, ADP-ribosylation factor-like protein 8B, Proton myo-inositol cotransporter, Ras-related protein Rab-6A, Tetraspanin-6, Claudin-10, Claudin-2, Claudin-3, GDP-mannose 4.6 dehydratase, Glucosamine-6-phosphate isomerase 1, Lysosome membrane protein 2, Major facilitator superfamily domain-containing protein 12, Melanophilin, Sepiapterin reductase, Thioredoxin domain-containing protein 17, 3-hydroxybutyrate dehydrogenase type 2, Calmodulin, Carboxypeptidase Q, Flotillin-2, Galectin-3-binding protein, P2X purinoceptor 4, Protein dopey-2, Protein S100-A6, Ras-related protein Rab-35, Serine/threonine-protein phosphatase 2A catalytic subunit alpha iso form, 2′-deoxynucleoside 5′-phosphate N-hydrolase 1, Acid ceramidase, Calbindin, CD59 glycoprotein, CD81 antigen, Cytochrome b561, Enolase-phosphatase E1, Golgi phosphoprotein 3, Nicastrin, Probable serine carboxypeptidase CPVL, Ragulator complex protein LAMTORS, Ras-related protein Rab-27B, Secretory carrier-associated membrane protein 2, Spermine synthase, S-phase kinase-associated protein 1, Transmembrane 7 superfamily member 3, Tumor protein D52, Ubiquitin-conjugating enzyme E2 variant 2, UDP-glucose 6-dehydrogenase, Zinc-alpha-2-glycoprotein, Glycerophosphodiester phosphodiesterase domain-containing protein 3, EF-hand domain-containing protein D2, Ras-related protein Rab-14, Omega-amidase NIT2, Alpha-actinin-1, Monocarboxylate transporter 5, Ras-related protein Rab-12, Ras-related protein Rab-8A, Transmembrane protein 63A, Beta-2-microglobulin, V-type proton ATPase subunit d 1, Lipid phosphate phosphohydrolase 1, Integral membrane protein GPR155, 14-3-3 protein sigma, E3 ubiquitin-protein ligase LRSAM1, HLA class II histocompatibility antigen. DM alpha chain, Ras-related protein Rab-9A, Src substrate cortactin, Aquaporin-7, Gamma-synuclein, 14-3-3 protein theta, Aspartate aminotransferase. cytoplasmic, Chloride intracellular channel protein 3, Destrin, GTPase HRas, Prostaglandin reductase 2, T-complex protein 1 subunit epsilon, Inter-alpha-trypsin inhibitor heavy chain H4, Aldehyde dehydrogenase family 1 member A3, Annexin A3, Battenin, Cathepsin D, N(G).N(G)-dimethylarginine dimethylaminohydrolase 1, Neural proliferation differentiation and control protein 1, Proactivator polypeptide, Prostate-specific antigen, Protein lifeguard 3, Protein Niban, Protein spinster homolog 1, Ragulator complex protein LAMTOR2, Ragulator complex protein LAMTOR3, Synaptotagmin-7, Transmembrane protein 106B, Unconventional myosin-Vc, Vesicle-associated membrane protein 2, V-type proton ATPase subunit F, A-kinase anchor protein 7 isoforms alpha and beta, Arylsulfatase F, C-Jun-amino-terminal kinase-interacting protein 4, Deleted in malignant brain tumors 1 protein, Glia maturation factor beta, Glutamate carboxypeptidase 2, Glutathione synthetase, Hippocalcin-like protein 1, Myristoylated alanine-rich C-kinase substrate, NAD(P)H-hydrate epimerase, Napsin-A, Phosphoacetylglucosamine mutase, Probable phospholipid-transporting ATPase IA, Quinone oxidoreductase, Retinol-binding protein 5, Serine incorporator 2, Solute carrier family 15 member 1, Sorcin, Sucrase-isomaltase. Intestinal, Actin-related protein ⅔ complex subunit 4, Actin-related protein ⅔ complex subunit 5, Adenine phosphoribosyltransferase, Costars family protein ABRACL, Ig alpha-1 chain C region, Interferon-induced transmembrane protein 3, Lactotransferrin, Purine nucleoside phosphorylase, Syntaxin-binding protein 4, Tetraspanin-8, Vacuolar protein-sorting-associated protein 25, Voltage-dependent anion-selective channel protein 1, Collagen alpha-1(VI) chain, Signal transducing adapter molecule 1, CD9 antigen, Flotillin-1, Grancalcin, Mannose-1-phosphate guanyltransferase beta, Proteasome subunit alpha type-7, Ras-related protein Rab-18, Vacuolar protein sorting-associated protein 37C, Signal transducing adapter molecule 2, Pancreatic secretory granule membrane major glycoprotein GP2, 1-phosphatidylinosito14.5-bisphosphate phosphodiesterase gamma-2, Lysosome-associated membrane glycoprotein 2, Cysteine-rich C-terminal protein 1, Plasma serine protease inhibitor, Tyrosine-protein kinase Lck, Ragulator complex protein LAMTOR4, Secretory carrier-associated membrane protein 1, Peptidyl-prolyl cis-trans isomerase FKBP1A, Transmembrane protein 176A, Thymosin beta-4, Haloacid dehalogenase-like hydrolase domain-containing protein 2, Cell division control protein 42 homolog, Ras-related protein Rab-17, Chloride intracellular channel protein 6, Choline transporter-like protein 4, Flavin reductase (NADPH), Ras-related protein Rab-10, Heme-binding protein 2, Fatty acid-binding protein. epidermal, Small integral membrane protein 5, Lysosomal-associated transmembrane protein 4A, Phytanoyl-CoA dioxygenase domain-containing protein 1, Proteasome subunit alpha type-5, Calmodulin-like protein 3, Presenilin-1, Ribosyldihydronicotinamide dehydrogenase [quinone], Translationally-controlled tumor protein, Lysosome-associated membrane glycoprotein 1, ADP-ribosyl cyclase 1, Myotrophin, Dynein light chain 2. cytoplasmic, Dihydropteridine reductase, Nicotinate-nucleotide pyrophosphorylase [carboxylating], Cell cycle control protein 50A, Phosphomevalonate kinase, Eukaryotic translation initiation factor 4H, Protein tweety homo log 3, Sodium-dependent phosphate transport protein 2B, Ig lambda-2 chain C regions, Cellular retinoic acid-binding protein 2, Protein CutA, Proteasome subunit alpha type-4, Solute carrier family 35 member F6, Delta-aminolevulinic acid dehydratase, L-xylulose reductase, Uroplakin-1a, Cornifin-A, Zinc finger protein 185, Transmembrane protein 8A, Prenylcysteine oxidase 1, Lysozyme C, Paralemmin-1, Carcinoembryonic antigen-related cell adhesion molecule 6, Sodium/glucose cotransporter 1, Prostaglandin reductase 1, Protein S100-A9, MICAL-like protein 1, Alpha/beta hydrolase domain-containing protein 14B, Aquaporin-2, Glutathione S-transferase P, Probable almitoyltransferase ZDHHC1, Ras-related protein Rab-8B, Transmembrane protease serine 2, Ras-related protein Rab-1B, Ras-related protein Rab-1A, Ras-related protein Rab-43, Synaptogyrin-2, HLA class II histocompatibility antigen. DRB1-15 beta chain, Lysosomal protein NCU-G1, Calcium-binding protein 39, Dynamin-2, CDC42 small effector protein 2, Ferritin heavy chain, Solute carrier family 35 member F2,Probable hydrolase PNKD, Cathepsin Z, Tubulin beta-2B chain, Thiosulfate sulfurtransferase/rhodanese-like domain-containing protein 1, Mitogen-activated protein kinase 1, Alcohol dehydrogenase class-3, Low molecular weight phosphotyrosine protein phosphatase, Annexin A4, Septin-2, Glutathione S-transferase Mu 3, Protein BRICK1, Proteasome subunit beta type-2, Ubiquitin- conjugating enzyme E2 K, Protein S100-A1, Microtubule-associated protein 1A, Glutathione S-transferase Mu 1, Matrix metalloproteinase-24, Small integral membrane protein 22, Heparan-alpha-glucosaminide N-acetyltransferase, Specifically androgen-regulated gene protein, Abl interactor 1, Uncharacterized protein C6orf132, ADP-ribosylation factor 5, Isocitrate dehydrogenase [NADP] cytoplasmic, Glycolipid transfer protein, Tropomyosin alpha-4 chain and Natural resistance-associated macrophage protein 2;
wherein said sample comprises urinary exosomes and wherein said sample has been obtained from said subject; and
wherein an altered level in said sample of one or more of said polypeptides in comparison to a control level is indicative of prostate cancer in said subject.

The features and discussion herein in relation to other aspects of the invention (e.g. in relation to preferred polypeptides or combinations thereof discussed above) apply, mutatis mutandis, to this aspect of the invention.

An altered level of one or more of the polypeptides as described herein includes any measurable alteration or change of the polypeptide (protein/biomarker) in question when the polypeptide in question is compared with a control level. An altered level includes an increased or decreased level. Preferably, the level is significantly altered, compared to the level found in an appropriate control sample or subject. More preferably, the significantly altered levels are statistically significant, preferably with a probability value of <0.05. Exemplary altered levels are discussed above in relation to “increased” and “decreased” levels.

In some aspects, methods of the invention are provided which further comprise a step of treating prostate cancer by therapy (e.g. pharmaceutical therapy) or surgery (e.g. prostatectomy). For example, if the result of a method of the invention is indicative of the prostate cancer in the subject (e.g. a postive diagnosis of prostate cancer is made), then an additional step of treating prostate cancer by therapy or surgery can be performed. Methods of treating prostate cancer by therapy or surgery are known in the art.

The invention will be further described with reference to the following non-limiting Example with reference to the following drawings in which:

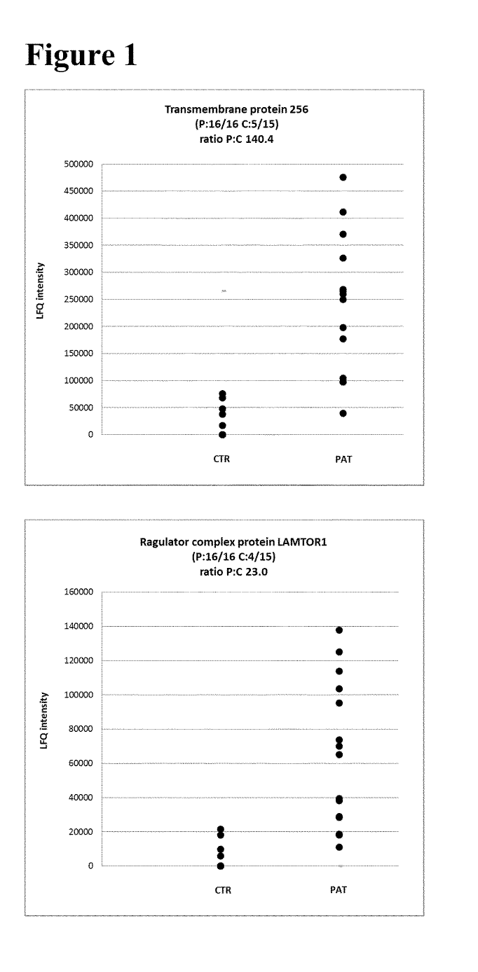

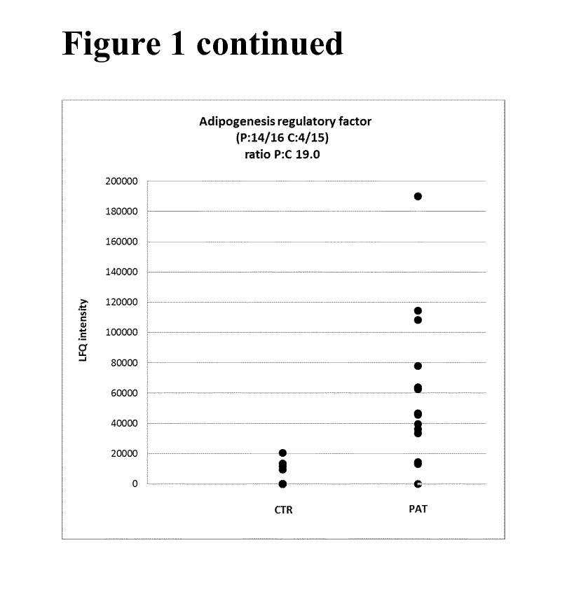

FIG. 1: Scatterplots, displaying the range of values observed in the PAT and CTR samples, are shown for illustrative purposes for the three biomarkers with the highest individual sensitivities at the chosen threshold (FIG. 1). For each sample type, PAT and CTR, the intensity (TOP3TIC) was plotted displaying the distribution along the y-axis. The iBAQ ratio and the number of samples (PAT and CTR) in which the protein was detected are also shown. LFQ: Label Free Quantification.

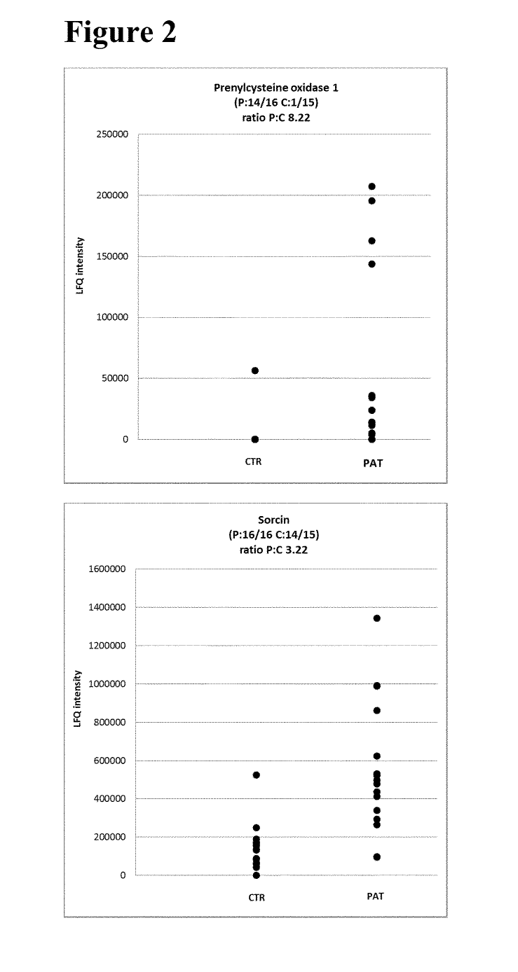

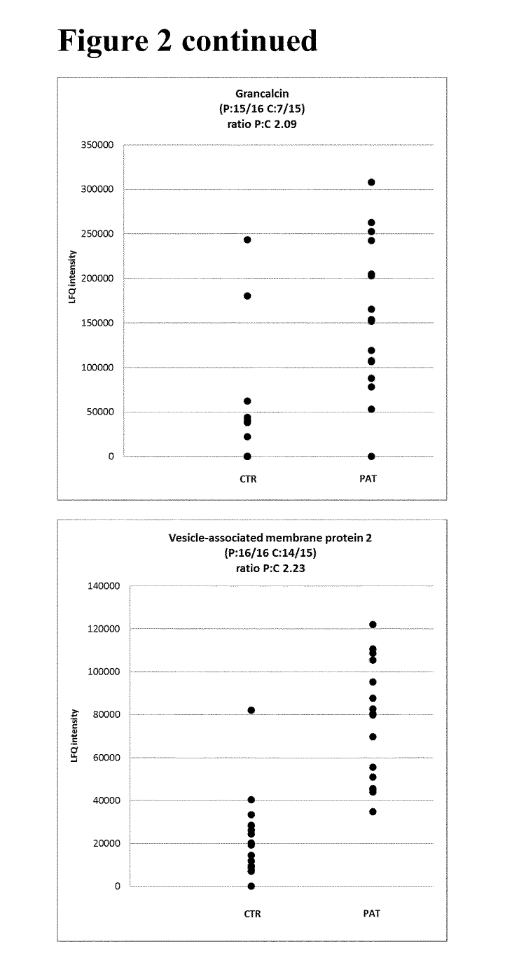

FIG. 2: Scatterplots, displaying the range of values observed in the PAT and CTR samples for Vesicle-associated membrane protein 2, Prenylcysteine oxidase 1, Sorcin and Grancalcin, are shown.

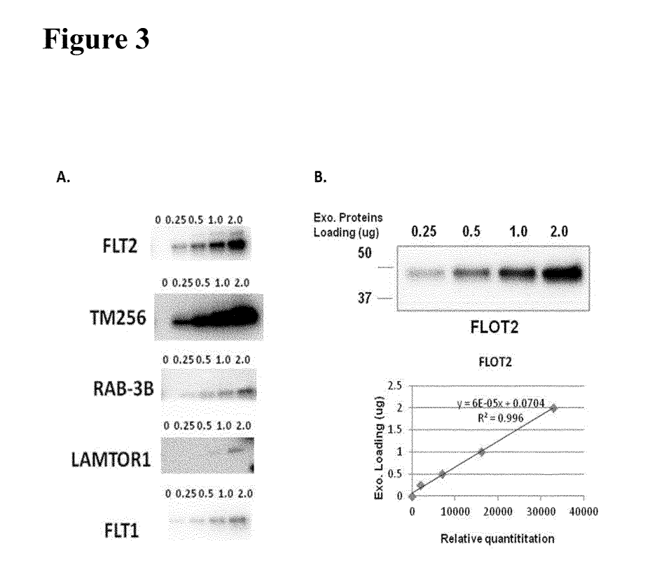

FIG. 3: Amount of exosomal protein required to detect specific biomarkers by Western blot. A. Different amounts of exosmal proteins were loaded on gels and specific proteins were detected by Western blot. B. Example where the quantity of the protein band (flotillin2 used as an example) is related to the amount of loaded urinary exosomes. FLOT2: flotillin2.

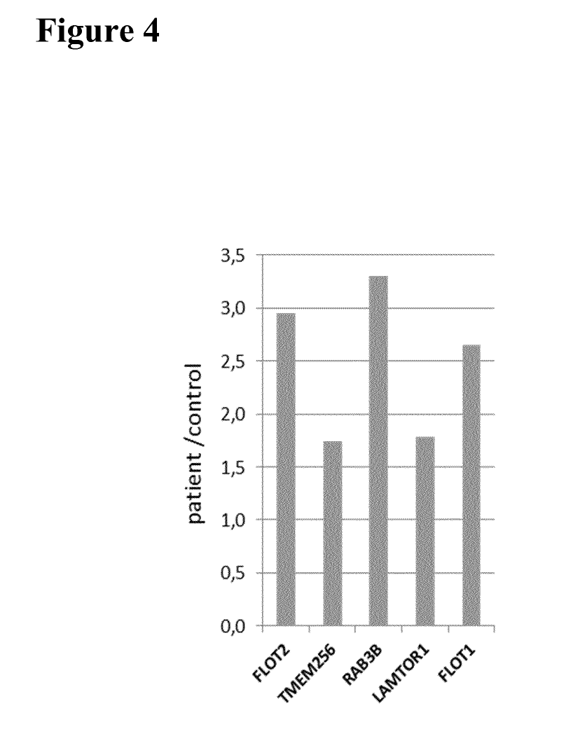

FIG. 4: Patient (prostate cancer patient) to control ratios for the different proteins (biomarkers) based on Western blot detection of specific biomarkers in urinary exosomes of healthy males and prostate cancer patients.

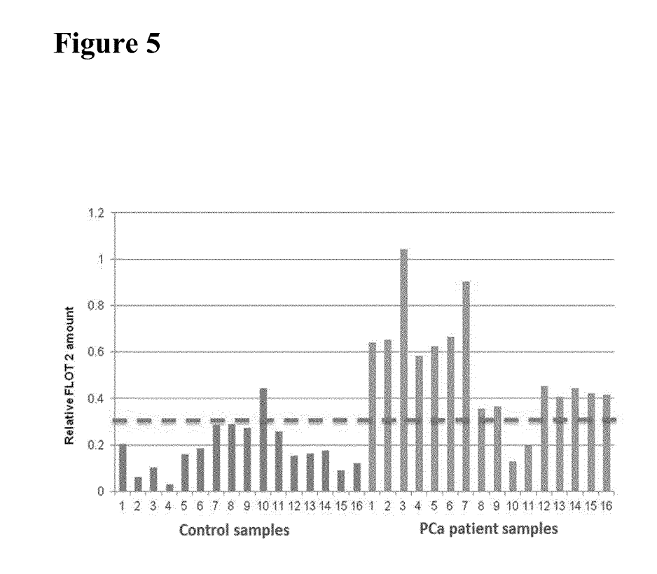

FIG. 5: Analysis of the diagnostic properties of flotillin2 based on Western blot detection. FLOT2: flotillin2.

### EXAMPLES

### Example 1

Results

Urinary exosomes from 15 healthy controls (CTR, C) and 17 prostate cancer patients (PAT, P) were isolated by serial centrifugation. In order to find exosomal proteins differently expressed in control versus prostate cancer patients, urinary exosomes were in-solution digested and analyzed using nanocapillary liquid chromatography-tandem mass spectrometry (nano-LC-MS/MS). This approach (termed “discovery analysis”) identified on average 1090 proteins per sample with 1% FDR. One patient sample, P11, was excluded from further analysis based on a much lower level of detectable proteins than in the other samples. Thus the comparison was performed on 15 control and 16 prostate cancer samples. The significantly differentially expressed proteins are summarized in Table 1, detailing the different annotations for the proteins. The vast majority of these proteins (221) were up-regulated in exosomes from prostate cancer, while a few (25) were down-regulated.

A ratio based on precursor ion intensity for top 3 total ion chromatograms (TOP3TIC) showing the enrichment of proteins in prostate cancer samples was calculated. The protein level of the samples was validated by pooling them into three sets of patient exosomes (PAT) and three sets of control exosomes (CTR) that were then subjected to LC/MS/MS with internal standard (iBAQ-intensity based absolute quantification) (Rosenberger G, Ludwig C, Rost H L, Aebersold R, & Malmstrom L (2014) aLFQ: an R-package for estimating absolute protein quantities from label-free LC-MS/MS proteomics data. Bioinformatics., 30, 2511-2513). The ratios PAT versus CTR (iBAQ) are presented in Table 2 (bold: significant difference p<0.05 in both analyses; normal font: significant only in discovery analysis). For the 21 proteins that were not detected in the validation study, the ratio obtained in the discovery analysis (TOP3TIC ratio) is shown in italics. The abundance of the proteins (ppm of total proteome) is also shown in Table 2, along with the number of patient or control samples in which the protein was confidently detected. Abundance values denote amounts found in the highest expression group (PAT for proteins overexpressed in prostate cancer, CTR for proteins underexpressed in prostate cancer).

A particularly good biomarker is characterized by having a high specificity and sensitivity for a specific condition. To identify the most promising biomarker candidates among the differentially expressed proteins, a tentative diagnostic call threshold was set for each individual protein to ensure 100% specificity (no erroneous positive call for CTR samples). The associated sensitivity levels were then calculated, and are detailed in Table 2 for each biomarker candidate. The proteins were analysed according to how many of the following criteria they met: (1) detected in validation study, (2) significantly altered in the validation study, (3) sensitivity above 40%, and (4) ratio PAT versus CTR above 1.75. The proteins found in Table 2 are first sorted by the number of criteria that they passed (more to less) and then by sensitivity (high to low). In relation to criteria (4), it is biomarkers whose level is increased in prostate cancer patients (samples) versus control that can have a PAT (patient) versus CTR (control) ratio of above 1.75. Analogously, for biomarkers whose level is decreased in prostate cancer patients (samples) versus control, an analogous criteria (4) may be applied, in which there is at least 1.75 times less expression of the biomarker in PAT versus CTR.

It was found that 58 proteins passed all 4 criteria. Data displaying the diagnostic call associated with each marker and patient sample for this focus list of promising biomarkers, based on the abovementioned specificity-driven diagnostic threshold, is presented in Table 3. Interestingly, 17 of the biomarkers displayed individual sensitivities above 60%, of which the highest sensitivity, at 94%, was observed for Sequence 1 (Uniprot entry name TM256_HUMAN—see Table 2).

Scatterplots, displaying the range of values observed in the PAT and CTR samples, are shown for illustrative purposes for the three biomarkers with the highest individual sensitivities at the chosen threshold (FIG. 1). For each sample type, PAT and CTR, the intensity (TOP3TIC) was plotted displaying the distribution along the y-axis. The iBAQ ratio and the number of samples (PAT and CTR) in which the protein was detected are also shown.

The proteomic profile of exosomes from the prostate cancer cell line PC-3 has previously been described (Sandvig K & Llorente A (2012) Proteomic analysis of microvesicles released by the human prostate cancer cell line PC-3. Mol. Cell Proteomics., 11, M111.012914). Only 29 proteins from Table 2 and 7 proteins from the more focused biomarker candidate list in Table 3 were common to the previously defined list of PC-3 exosomal proteins. These common proteins are summarised in Table 4.

All the biomarkers with individual diagnostic sensitivity above 60% (Sequences 1-17, see Table 2)), with the exception of Sequences 4, 14 and 16, could be combined with one other biomarker from Table 3 to provide full sensitivity and specificity for a two-marker test (assuming a positive call for at least one marker is sufficient for an overall positive diagnostic call). Sequences 20, 35 and 56, although of lower individual sensitivity, could also be combined with only one more marker (Sequence 1) to provide the same full diagnostic accuracy. The same could be achieved for Sequences 4, 14 and 16 in various three-marker combinations. Some examples of the 2- and 3-marker combinations providing full differentiation between prostate cancer and control samples are listed in Table 5. Other appropriate combinations are easily derived from the data in Table 3.

Other appropriate combinations of markers can be derived from Table 3 by combining two or more of the markers in Table 3 (e.g. 2, 3, 4, 5 or 6 markers, preferably 2, 3 or 4 markers) that results in one or more of the patients (P) (preferably the majority of the patients, e.g. 9, 10, 11, 12, 13, 14, 15 or 16 of the patients, more preferably all of the patients) being associated with a positive call (as indicated by a “1” in Table 3) from at least one marker in the combination. Put another way, other appropriate combinations of markers (sequences/polypeptides) can be derived from Table 3 by combining two or more of the sequence rows (e.g. 2, 3, 4, 5 or 6 sequence rows, preferably 2, 3 or 4 sequence rows) such that the combination of said two or more sequence rows has at least one positive call (as indicated by “1”) in one or more patient columns (P) (preferably the majority of the patient columns, e.g. 9, 10, 11, 12, 13, 14, 15 or 16 of the patient columns, more preferably all of the patient columns). By way of example, sequences (markers) 12, 13 and 14 would be an appropriate three-marker combination as when sequence rows 12, 13 and 14 are combined there is at least one positive call (“1”) in all of the patient columns (P).

Although as few as 1, 2 or 3 of the abovementioned biomarkers may be sufficient, more markers may be added to increase technical robustness. Furthermore, for some applications, the overall diagnostic call threshold for an expanded panel comprising the abovementioned markers may be set to require more than one positive call for the individual markers within the panel. This will reduce the rate of false positive diagnostic calls. A test requiring two independently positive markers for an overall positive diagnostic call, can still achieve full sensitivity with a combination of only four markers (an illustrative example, combining Sequences 1, 2, 3 and 9, is shown in Table 5). The diagnostic input from the individual markers in a panel may also be incorporated in an algorithm to provide a score, to be compared to a diagnostic threshold score.

The abovementioned analysis was based on setting a diagnostic threshold to provide 100% specificity (i.e. all control patient samples would be below the set threshold). One could also envision setting the appropriate diagnostic threshold to ensure maximum combined sensitivity and specificity. Table 6 shows the top ranking protein markers (those with a combined sensitivity and specificity of at least 160%) when performing such analysis. This alternative focus list of potential biomarkers displays some differences from the focus list of Table 3, which was developed based on specificity-driven diagnostic thresholds. Among 11 proteins in table 6 with a combined sensitivity and specificity above 170%, four were not included in table 3; Vesicle-associated membrane protein 2, Prenylcysteine oxidase 1, Sorcin and Grancalcin.

Scatterplots, displaying the range of values observed in the PAT and CTR samples for these proteins, are provided in FIG. 2. Particularly noteworthy among these is Prenylcysteine oxidase 1, which has an extreme outlier among the control samples, which dramatically reduces the sensitivity when setting a specificity-driven threshold. Discarding this outlier, which could very well be a non-diagnosed case with underlying disease, the protein is a very good discriminator between control samples and confirmed prostate cancer patients. Thus, Prenylcysteine oxidase 1 as well as other proteins in table 6 can be used as diagnostic biomarkers, and may be combined in a diagnostic test with any of the abovementioned proteins.

Experimental Procedures

Urine Samples

Urine samples were collected either from healthy control (15 samples) or from prostate cancer patients (17 samples) the day before prostatectomy. Samples were collected during the morning and were processed within 2 hours. The urine pH and the presence of leukocytes, nitrites, proteins, glucose, ketones and blood were analyzed with a Combur⁷ strip-Test strip in an Urysis1100 urine analyzer (Roche Diagnostics). Creatinine was measured with a creatinine urinary detection kit (Arbor assays). The collection of urine samples was approved by the Norwegian Regional Committees for medical and health research ethics.

Exosome Isolation

Urinary exosomes were isolated by serial centrifugation. Briefly, urine was centrifuged at 2,000 g for 15 min, and then at 10,000 g for 30 min discarding the pellet at each step. The exosomes present in the supernatant were then pelleted at 100,000 g for 70 min and washed with PBS. Exosomes were then resuspended again in PBS, filtrated through a 200 nm pore filter and pelleted at 100,000 g for 70 min. The pellet was resuspended in 50-100 μ1 PBS and stored at −80 ° C.

Protein Measurements

The amount of protein in exosomes was determined using a BCA assay kit (Pierce, Thermo Scientific) according to the manufacturer's instructions. BSA was used as standard protein.

In-solution Digestion of Exosomes

Exosomes (2 μg) in one volume of PBS were mixed with four volumes of cold acetone (with 1M HCl) and methanol at −20 ° C. The samples were centrifuged at 15,000× g for 15 min and the pellets were dried in a Speed-Vac instrument. Then, the pellets were dissolved in 50 μl of a fresh solution of 100 mM ammonium bicarbonate with 6 M urea, and subsequently reduced with 10 mM dithiothreitol at 30° C. for 30 min. The samples were then incubated with 25 mM iodoacetamide to alkylate exposed side chains for 1 h at room temperature away from light. The enzymatic digestion was initiated by adding 1 μg Lys-C to the samples and incubating them at 37° C. for 2 hours. Finally, 240 μl 50 mM ammonium bicarbonate with 10 μg trypsin was added and the samples were first incubated for 1 h at 37 ° C., followed by 15 h at 30° C. Prior to LC-MS analysis, formic acid (5 μl ) was added to the digested exosomes.

Mass Spectrometry Analyses

For MS analyses, the samples (one quarter of the volume, 0.5 μg) were injected into an Ultimate 3000 nanoLC system (Dionex, Sunnyvale Calif., USA) connected to a linear quadrupole ion trap-orbitrap (LTQ-Orbitrap XL) mass spectrometer (ThermoScientific, Bremen, Germany) equipped with a nanoelectrospray ion source. An Acclaim PepMap 100 column (C18, 3 μm, 100 Å) (Dionex) with a capillary of 25 cm bed length was used for separation by liquid chromatography. A flow rate of 300 nl/min was employed with a solvent gradient of 4% B to 60% B in 230 min. Solvent A was 0.1% formic acid, whereas aqueous 90% acetonitrile in 0.1% formic acid was used as solvent B. The mass spectrometer was operated in the data-dependent mode to automatically switch between Orbitrap-MS and. LTQ-MS/MS acquisition. Survey full scan MS spectra (from m/z 300 to 2000) were acquired in the Orbitrap with resolution R=60,000 at m/z 400 (after accumulation to a target of 500,000 charges in the LTQ). The method used allowed sequential isolation of the most intense ions, up to six, depending on signal intensity, for fragmentation on the linear ion trap using collision induced dissociation at a target value of 10,000 charges.

To validate the quantitative analyses for the complete data set, the samples (aliquots of the digested exosomes that were used in the previous analysis) were pooled into three sets of patient exosomes and three sets of controls (aliquots of digested exosomes and subjected to LC/MS/MS with internal standard (iBAQ-intensity based absolute quantification (Rosenberger G, Ludwig C, Rost H L, Aebersold R, & Malmstrom L (2014) aLFQ: an R-package for estimating absolute protein quantities from label-free LC-MS/MS proteomics data. Bioinformatics., 30, 2511-2513). The samples were separated on the Dionex U3000 capillary/nano-HPLC system (Dionex, Sunnyvale, Calif.), which was directly interfaced with a Thermo Fisher Q Exactive Orbitrap mass spectrometer. The mass spectrometer was operated in the data-dependent acquisition mode using the Xcalibur 2.2 software. Single MS full-scan in the Orbitrap (300-1750 m/z, 70,000 resolution at m/z 200, AGC target 1e6, maximum injection time 20 ms) was followed by 10 data-dependent MS/MS scans in the Orbitrap after accumulation of 1e6 ions in the C-trap or an injection time of 120 ms (fixed injection time method) at 35,000 resolution (isolation width 2.0 or 3.0 mlz, underfill ratio 0.1%, dynamic exclusion 20 or 45 s) at 25 or 30% normalized collision energy. Proteins that were present only in 1 of the 3 sets were considered invalid.

Data Processing

Tandem mass spectra were extracted, charge state deconvoluted and deisotoped by [Peptide Finder] version [1.8.1]. All MS/MS samples were analyzed using Mascot (Matrix Science, London, UK; version 2.4.0). Mascot was set up to search the UniProt database (selected for Homo sapiens, ver 14.05.2014 version, 20279 entries) assuming the digestion enzyme trypsin. Mascot was searched with a fragment ion mass tolerance of 0.60 Da and a parent ion tolerance of 10.0 ppm. Carbamidomethyl of cysteine was specified in Mascot as a fixed modification. Oxidation of methionine, acetylation of the N-terminus and phosphorylation of serine, threonine and tyrosine were specified in Mascot as variable modifications. Scaffold (version Scaffold 4.3.2, Proteome Software Inc., Portland, Oreg.) was used to validate MS/MS based peptide and protein identifications. Peptide identifications were accepted if they could be established at greater than 95.0% probability by the Peptide Prophet algorithm (Keller A, Nesvizhskii A I, Kolker E, & Aebersold R (2002) Empirical statistical model to estimate the accuracy of peptide identifications made by MS/MS and database search. Anal. Chem., 74, 5383-5392) with Scaffold delta-mass correction. Protein identifications were accepted if they could be established at greater than 99.0% probability and contained at least 1 identified peptide. Protein probabilities were assigned by the Protein Prophet algorithm (Nesvizhskii, Al et al Anal. Chem. 2003;75(17):4646-58). Proteins that contained similar peptides and could not be differentiated based on MS/MS analysis alone were grouped to satisfy the principles of parsimony. MS/MS spectra from protein hits identified with only 1 peptide were investigated manually.

Statistics

For comparing datasets Fisher's exact test (CI 95%) was used to determine significant changes between the subproteomes of exosomes from patients and healthy controls. The label-free quantitative measurement of individual samples used both PSM (peptide spectra match) and TOP3TIC (top 3 precursor intensities from total ion chromatogram) and only protein hits significantly altered (p<0.05) for both were considered. The suitability as biomarker for the candidate proteins were addressed by determining an intensity threshold in every sample. The intensity threshold was optimized to give maximum specificity and sensitivity of the test, i.e. to maximize the difference between true positives (TP>FN) and false negatives (TN>FP). This enabled us to produce a table displaying most promising candidates within the cohort.

TABLE 1
List of proteins found to be differently expressed in urinary exosomes from prostate cancer patients
compared to normal controls

| | Uniprot | Uniprot | |
|---|---|---|---|
| Sequence | Entry Name | Acc. No. | Full Protein Name |
| 1 | TM256_HUMAN | Q8N2U0 | Transmembrane protein 256 |
| 2 | ADIRF_HUMAN | Q15847 | Adipogenesis regulatory factor |
| 3 | LTOR1_HUMAN | Q6IAA8 | Ragulator complex protein LAMTOR1 |
| 4 | PLSL_HUMAN | P13796 | Plastin-2 |
| 5 | RAB2A_HUMAN | P61019 | Ras-related protein Rab-2A |
| 6 | RAB3B_HUMAN | P20337 | Ras-related protein Rab-3B |
| 7 | RAB3D_HUMAN | O95716 | Ras-related protein Rab-3D |
| 8 | RAB7A_HUMAN | P51149 | Ras-related protein Rab-7a |
| 9 | VATL_HUMAN | P27449 | V-type proton ATPase 16 kDa proteolipid subunit |
| 10 | STEA4_HUMAN | Q687X5 | Metalloreductase STEAP4 |
| 11 | PARK7_HUMAN | Q99497 | Protein DJ-1 |
| 12 | S1OOP_HUMAN | P25815 | Protein S100-P |
| 13 | SYTL4_HUMAN | Q96C24 | Synaptotagmin-like protein 4 |
| 14 | ARL8B_HUMAN | Q9NVJ2 | ADP-ribosylation factor-like protein 8B |
| 15 | MYCT_HUMAN | Q96QE2 | Proton myo-inositol cotransporter |
| 16 | RAB6A_HUMAN | P20340 | Ras-related protein Rab-6A |
| 17 | TSN6_HUMAN | O43657 | Tetraspanin-6 |
| 18 | CLD10_HUMAN | P78369 | Claudin-10 |
| 19 | CLD2_HUMAN | P57739 | Claudin-2 |
| 20 | CLD3_HUMAN | O15551 | Claudin-3 |
| 21 | GMDS_HUMAN | O60547 | GDP-mannose 4.6 dehydratase |
| 22 | GNPI1_HUMAN | P46926 | Glucosamine-6-phosphate isomerase 1 |
| 23 | SCRB2_HUMAN | Q14108 | Lysosome membrane protein 2 |
| 24 | MFS12_HUMAN | Q6NUT3 | Major facilitator superfamily domain-containing protein 12 |
| 25 | MELPH_HUMAN | Q9BV36 | Melanophilin |
| 26 | SPRE_HUMAN | P35270 | Sepiapterin reductase |
| 27 | TXD17_HUMAN | Q9BRA2 | Thioredoxin domain-containing protein 17 |
| 28 | BDH2_HUMAN | Q9BUT1 | 3-hydroxybutyrate dehydrogenase type 2 |
| 29 | CALM_HUMAN | P62158 | Calmodulin |
| 30 | CBPQ_HUMAN | Q9Y646 | Carboxypeptidase Q |
| 31 | FLOT2_HUMAN | Q14254 | Flotillin-2 |
| 32 | LG3BP_HUMAN | Q08380 | Galectin-3-binding protein |
| 33 | P2RX4_HUMAN | Q99571 | P2X purinoceptor 4 |
| 34 | DOP2_HUMAN | Q9Y3R5 | Protein dopey-2 |
| 35 | S10A6_HUMAN | P06703 | Protein S100-A6 |
| 36 | RAB35_HUMAN | Q15286 | Ras-related protein Rab-35 |
| 37 | PP2AA_HUMAN | P67775 | Serine/threonine-protein phosphatase 2A catalytic subunit |
| | | | alpha isoform |
| 38 | DNPH1_HUMAN | Q43598 | 2′-deoxynucleoside 5′-phosphate N-hydrolase 1 |
| 39 | ASAH1_HUMAN | Q13510 | Acid ceramidase |
| 40 | CALB1_HUMAN | P05937 | Calbindin |
| 41 | CD59_HUMAN | P13987 | CD59 glycoprotein |
| 42 | CD81_HUMAN | P60033 | CD81 antigen |
| 43 | CY561_HUMAN | P49447 | Cytochrome b561 |
| 44 | ENOPH_HUMAN | Q9UHY7 | Enolase-phosphatase E1 |
| 45 | GOLP3_HUMAN | Q9H4A6 | Golgi phosphoprotein 3 |
| 46 | NICA_HUMAN | Q92542 | Nicastrin |
| 47 | CPVL_HUMAN | Q9H3G5 | Probable serine carboxypeptidase CPVL |
| 48 | LTOR5_HUMAN | O43504 | Ragulator complex protein LAMTOR5 |
| 49 | RB27B_HUMAN | O00194 | Ras-related protein Rab-27B |
| 50 | SCAM2_HUMAN | O15127 | Secretory carrier-associated membrane protein 2 |
| 51 | SPSY_HUMAN | P52788 | Spermine synthase |
| 52 | SKP1_HUMAN | P63208 | S-phase kinase-associated protein 1 |
| 53 | TM753_HUMAN | Q9NS93 | Transmembrane 7 superfamily member 3 |
| 54 | TPD52_HUMAN | P55327 | Tumor protein D52 |
| 55 | UB2V2_HUMAN | Q15819 | Ubiquitin-conjugating enzyme E2 variant 2 |
| 56 | UGDH_HUMAN | O60701 | UDP-glucose 6-dehydrogenase |
| 57 | ZA2G_HUMAN | P25311 | Zinc-alpha-2-glycoprotein |
| 58 | GDPD3_HUMAN | Q7L5L3 | Glycerophosphodiester phosphodiesterase domain- |
| | | | containing protein 3 |
| 59 | EFHD2_HUMAN | Q96C19 | EF-hand domain-containing protein D2 |
| 60 | RAB14_HUMAN | P61106 | Ras-related protein Rab-14 |
| 61 | NIT2_HUMAN | Q9NQR4 | Omega-amidase NIT2 |
| 62 | ACTN1_HUMAN | P12814 | Alpha-actinin-1 |
| 63 | MOT5_HUMAN | O15374 | Monocarboxylate transporter 5 |
| 64 | RAB12_HUMAN | Q6IQ22 | Ras-related protein Rab-12 |
| 65 | RAB8A_HUMAN | P61006 | Ras-related protein Rab-8A |
| 66 | TM63A_HUMAN | O94886 | Transmembrane protein 63A |
| 67 | B2MG_HUMAN | P61769 | Beta-2-microglobulin |
| 68 | VA0D1_HUMAN | P61421 | V-type proton ATPase subunit d 1 |
| 69 | LPP1_HUMAN | O14494 | Lipid phosphate phosphohydrolase 1 |
| 70 | GP155_HUMAN | Q7Z3F1 | Integral membrane protein GPR155 |
| 71 | 1433S_HUMAN | P31947 | 14-3-3 protein sigma |
| 72 | LRSM1_HUMAN | Q6UWE0 | E3 ubiquitin-protein ligase LRSAM1 |
| 73 | DMA_HUMAN | P28067 | HLA class 11 histocompatibility antigen. DM alpha chain |
| 74 | RAB9A_HUMAN | P51151 | Ras-related protein Rab-9A |
| 75 | SRC8_HUMAN | Q14247 | Src substrate cortactin |
| 76 | AQP7_HUMAN | O14520 | Aquaporin-7 |
| 77 | SYUG_HUMAN | O76070 | Gamma-synuclein |
| 78 | 1433T_HUMAN | P27348 | 14-3-3 protein theta |
| 79 | AATC_HUMAN | P17174 | Aspartate aminotransferase. cytoplasmic |
| 80 | CLIC3_HUMAN | O95833 | Chloride intracellular channel protein 3 |
| 81 | DEST_HUMAN | P60981 | Destrin |
| 82 | RASH_HUMAN | P01112 | GTPase HRas |
| 83 | PTGR2_HUMAN | Q8N8N7 | Prostaglandin reductase 2 |
| 84 | TCPE_HUMAN | P48643 | T-complex protein 1 subunit epsilon |
| 85 | ITIH4_HUMAN | Q14624 | Inter-alpha-trypsin inhibitor heavy chain H4 |
| 86 | AL1A3_HUMAN | P47895 | Aldehyde dehydrogenase family 1 member A3 |
| 87 | ANXA3_HUMAN | P12429 | Annexin A3 |
| 88 | CLN3_HUMAN | Q13286 | Battenin |
| 89 | CATD_HUMAN | P07339 | Cathepsin D |
| 90 | DDAH1_HUMAN | O94760 | N(G).N(G)-dimethylarginine dimethylaminohydrolase 1 |
| 91 | NPDC1_HUMAN | Q9NQX5 | Neural proliferation differentiation and control protein 1 |
| 92 | SAP_HUMAN | P07602 | Proactivator polypeptide |
| 93 | KLK3_HUMAN | P07288 | Prostate-specific antigen |
| 94 | LFG3_HUMAN | Q969X1 | Protein lifeguard 3 |
| 95 | NIBAN_HUMAN | Q9BZQ8 | Protein Niban |
| 96 | SPNS1_HUMAN | Q9H2V7 | Protein spinster homolog 1 |
| 97 | LTOR2_HUMAN | Q9Y2Q5 | Ragulator complex protein LAMTOR2 |
| 98 | LTOR3_HUMAN | Q9UHA4 | Ragulator complex protein LAMTOR3 |
| 99 | SYT7_HUMAN | O43581 | Synaptotagmin-7 |
| 100 | T106B_HUMAN | Q9NUM4 | Transmembrane protein 106B |
| 101 | MYO5C_HUMAN | Q9NQX4 | Unconventional myosin-Vc |
| 102 | VAMP2_HUMAN | P63027 | Vesicle-associated membrane protein 2 |
| 103 | VATF_HUMAN | Q16864 | V-type proton ATPase subunit F |
| 104 | AKA7A_HUMAN | O43687 | A-kinase anchor protein 7 isoforms alpha and beta |
| 105 | ARSF_HUMAN | P54793 | Arylsulfatase F |
| 106 | JIP4_HUMAN | O60271 | C-Jun-amino-terminal kinase-interacting protein 4 |
| 107 | DMBT1_HUMAN | Q9UGM3 | Deleted in malignant brain tumors 1 protein |
| 108 | GMFB_HUMAN | P60983 | Glia maturation factor beta |
| 109 | FOLH1_HUMAN | Q04609 | Glutamate carboxypeptidase 2 |
| 110 | GSHB_HUMAN | P48637 | Glutathione synthetase |
| 111 | HPCL1_HUMAN | P37235 | Hippocalcin-like protein 1 |
| 112 | MARCS_HUMAN | P29966 | Myristoylated alanine-rich C-kinase substrate |
| 113 | NNRE_HUMAN | Q8NCW5 | NAD(P)H-hydrate epimerase |
| 114 | NAPSA_HUMAN | O96009 | Napsin-A |
| 115 | AGM1_HUMAN | O95394 | Phosphoacetylglucosamine mutase |
| 116 | AT8A1_HUMAN | Q9Y2Q0 | Probable phospholipid-transporting ATPase IA |
| 117 | QOR_HUMAN | Q08257 | Quinone oxidoreductase |
| 118 | RET5_HUMAN | P82980 | Retinol-binding protein 5 |
| 119 | SERC2_HUMAN | Q96SA4 | Serine incorporator 2 |
| 120 | S15A1_HUMAN | P46059 | Solute carrier family 15 member 1 |
| 121 | SORCN_HUMAN | P30626 | Sorcin |
| 122 | SUIS_HUMAN | P14410 | Sucrase-isomaltase. intestinal |
| 123 | ARPC4_HUMAN | P59998 | Actin-related protein 2/3 complex subunit 4 |
| 124 | ARPC5_HUMAN | O15511 | Actin-related protein 2/3 complex subunit 5 |
| 125 | APT_HUMAN | P07741 | Adenine phosphoribosyltransferase |
| 126 | ABRAL_HUMAN | Q9P1F3 | Costars family protein ABRACL |
| 127 | IGHA1_HUMAN | P01876 | Ig alpha-1 chain C region |
| 128 | IFM3_HUMAN | Q01628 | Interferon-induced transmembrane protein 3 |
| 129 | TRFL_HUMAN | P02788 | Lactotransferrin |
| 130 | PNPH_HUMAN | P00491 | Purine nucleoside phosphorylase |
| 131 | STX134_HUMAN | Q6ZWJ1 | Syntaxin-binding protein 4 |
| 132 | TSN8_HUMAN | P19075 | Tetraspanin-8 |
| 133 | VPS25_HUMAN | Q9BRG1 | Vacuolar protein-sorting-associated protein 25 |
| 134 | VDAC1_HUMAN | P21796 | Voltage-dependent anion-selective channel protein 1 |
| 135 | CO6A1_HUMAN | P12109 | Collagen alpha-1(VI) chain |
| 136 | STAM1_HUMAN | Q92783 | Signal transducing adapter molecule 1 |
| 137 | CD9_HUMAN | P21926 | CD9 antigen |
| 138 | FLOT1_HUMAN | O75955 | Flotillin-1 |
| 139 | GRAN_HUMAN | P28676 | Grancalcin |
| 140 | GMPPB_HUMAN | Q9Y5P6 | Mannose-1-phosphate guanyltransferase beta |
| 141 | PSA7_HUMAN | O14818 | Proteasome subunit alpha type-7 |
| 142 | RAB18_HUMAN | Q9NP72 | Ras-related protein Rab-18 |
| 143 | VP37C_HUMAN | A5D8V6 | Vacuolar protein sorting-associated protein 37C |
| 144 | STAM2_HUMAN | O75886 | Signal transducing adapter molecule 2 |
| 145 | GP2_HUMAN | P55259 | Pancreatic secretory granule membrane major glycoprotein |
| | | | GP2 |
| 146 | PLCG2_HUMAN | P16885 | 1-phosphatidylinositol 4.5-bisphosphate phosphodiesterase |
| | | | gamma-2 |
| 147 | LAMP2_HUMAN | P13473 | Lysosome-associated membrane glycoprotein 2 |
| 148 | CRCT1_HUMAN | Q9UGL9 | Cysteine-rich C-terminal protein 1 |
| 149 | IPSP_HUMAN | P05154 | Plasma serine protease inhibitor |
| 150 | LCK_HUMAN | P06239 | Tyrosine-protein kinase Lck |
| 151 | LTOR4 | Q0VGL1 | Ragulator complex protein LAMTOR4 |
| 152 | SCAM1_HUMAN | O15126 | Secretory carrier-associated membrane protein 1 |
| 153 | FKB1A_HUMAN | P62942 | Peptidyl-prolyl cis-trans isomerase FKBP1A |
| 154 | T176A_HUMAN | Q96HP8 | Transmembrane protein 176A |
| 155 | TYB4_HUMAN | P62328 | Thymosin beta-4 |
| 156 | HDHD2_HUMAN | Q9H0R4 | Haloacid dehalogenase-like hydrolase domain-containing |
| | | | protein 2 |
| 157 | CDC42_HUMAN | P60953 | Cell division control protein 42 homolog |
| 158 | RAB17_HUMAN | Q9H0T7 | Ras-related protein Rab-17 |
| 159 | CLIC6_HUMAN | Q96NY7 | Chloride intracellular channel protein 6 |
| 160 | CTL4_HUMAN | Q53GD3 | Choline transporter-like protein 4 |
| 161 | BLVRB_HUMAN | P30043 | Flavin reductase (NADPH) |
| 162 | RAB10_HUMAN | P61026 | Ras-related protein Rab-10 |
| 163 | HEBP2_HUMAN | Q9Y5Z4 | Heme-binding protein 2 |
| 164 | FABP5_HUMAN | Q01469 | Fatty acid-binding protein. epidermal |
| 165 | SMIM5_HUMAN | Q71RC9 | Small integral membrane protein 5 |
| 166 | LAP4A_HUMAN | Q15012 | Lysosomal-associated transmembrane protein 4A |
| 167 | PHYD1_HUMAN | Q5SRE7 | Phytanoyl-CoA dioxygenase domain-containing protein 1 |
| 168 | PSA5_HUMAN | P28066 | Proteasome subunit alpha type-5 |
| 169 | CALL3_HUMAN | P27482 | Calmodulin-like protein 3 |
| 170 | PSN1_HUMAN | P49768 | Presenilin-1 |
| 171 | NQ02_HUMAN | P16083 | Ribosyldihydronicotinamide dehydrogenase [quinone] |
| 172 | TCTP_HUMAN | P13693 | Translationally-control led tumor protein |
| 173 | LAMP1_HUMAN | P11279 | Lysosome-associated membrane glycoprotein 1 |
| 174 | CD38_HUMAN | P28907 | ADP-ribosyl cyclase 1 |
| 175 | MTPN_HUMAN | P58546 | Myotrophin |
| 176 | DYL2_HUMAN | Q96FJ2 | Dynein light chain 2. cytoplasmic |
| 177 | DHPR_HUMAN | P09417 | Dihydropteridine reductase |
| 178 | NADC_HUMAN | Q15274 | Nicotinate-nucleotide pyrophosphorylase [carboxylating] |
| 179 | CC50A_HUMAN | Q9NV96 | Cell cycle control protein 50A |
| 180 | PMVK_HUMAN | Q15126 | Phosphomevalonate kinase |
| 181 | IF4H_HUMAN | Q15056 | Eukaryotic translation initiation factor 4H |
| 182 | TTYH3_HUMAN | Q9C0H2 | Protein tweety homolog 3 |
| 183 | NPT2B_HUMAN | O95436 | Sodium-dependent phosphate transport protein 2B |
| 184 | LAC2_HUMAN (+1) | P0CG05 | Ig lambda-2 chain C regions |
| 185 | RABP2_HUMAN | P29373 | Cellular retinoic acid-binding protein 2 |
| 186 | CUTA_HUMAN | O60888 | Protein CutA |
| 187 | PSA4_HUMAN | P25789 | Proteasome subunit alpha type-4 |
| 188 | S35F6_HUMAN | Q8N357 | Solute carrier family 35 member F6 |
| 189 | HEM2_HUMAN | P13716 | Delta-aminolevulinic acid dehydratase |
| 190 | DCXR_HUMAN | Q7Z4W1 | L-xylulose reductase |
| 191 | UPK1A_HUMAN | O00322 | Uroplakin-1a |
| 192 | SPR1A_HUMAN | P35321 | Cornifin-A |
| 193 | ZN185_HUMAN | O15231 | Zinc finger protein 185 |
| 194 | TMM8A_HUMAN | Q9HCN3 | Transmembrane protein 8A |
| 195 | PCYOX_HUMAN | Q9UHG3 | Prenylcysteine oxidase 1 |
| 196 | LYSC_HUMAN | P61626 | Lysozyme C |
| 197 | PALM_HUMAN | O75781 | Paralemmin-1 |
| 198 | CEAM6_HUMAN | P40199 | Carcinoembryonic antigen-related cell adhesion molecule 6 |
| 199 | SC5A1_HUMAN | P13866 | Sodium/glucose cotransporter 1 |
| 200 | PTGR1_HUMAN | Q14914 | Prostaglandin reductase 1 |
| 201 | S10A9_HUMAN | P06702 | Protein S100-A9 |
| 202 | MILK1_HUMAN | Q8N3F8 | MICAL-like protein 1 |
| 203 | ABHEB_HUMAN | Q96IU4 | Alpha/beta hydrolase domain-containing protein 14B |
| 204 | AQP2_HUMAN | P41181 | Aquaporin-2 |
| 205 | GSTP1_HUMAN | P09211 | Glutathione 5-transferase P |
| 206 | ZDHC1_HUMAN | Q8WTX9 | Probable palmitoyltransferase ZDHHC1 |
| 207 | RAB8B_HUMAN | Q92930 | Ras-related protein Rab-8B |
| 208 | TMPS2_HUMAN | O15393 | Transmembrane protease serine 2 |
| 209 | RAB1B_HUMAN | Q9H0U4 | Ras-related protein Rab-1B |
| 210 | RAB1A_HUMAN | P62820 | Ras-related protein Rab-1A |
| 211 | RAB43_HUMAN | Q86YS6 | Ras-related protein Rab-43 |
| 212 | SNG2_HUMAN | O43760 | Synaptogyrin-2 |
| 213 | 2B1F_HUMAN | P01911 | HLA class II histocompatibility antigen. DRB1-15 beta |
| | | | chain |
| 214 | NCUG1_HUMAN | Q8WWB7 | Lysosomal protein NCU-G1 |
| 215 | CAB39_HUMAN | Q9Y376 | Calcium-binding protein 39 |
| 216 | DYN2_HUMAN | P50570 | Dynamin-2 |
| 217 | C42S2_HUMAN | Q9NRR3 | CDC42 small effector protein 2 |
| 218 | FRIH_HUMAN | P02794 | Ferritin heavy chain |
| 219 | S35F2_HUMAN | Q8IXU6 | Solute carrier family 35 member F2 |
| 220 | PNKD_HUMAN | Q8N490 | Probable hydrolase PNKD |
| 221 | CATZ_HUMAN | Q9UBR2 | Cathepsin Z |
| 222 | TBB2B_HUMAN | Q9BVA1 | Tubulin beta-2B chain |
| 223 | TSTD1_HUMAN | Q8NFU3 | Thiosulfate sulfurtransferase/rhodanese-like domain- |
| | | | containing protein 1 |
| 224 | MK01_HUMAN | P28482 | Mitogen-activated protein kinase 1 |
| 225 | ADHX_HUMAN | P11766 | Alcohol dehydrogenase class-3 |
| 226 | PPAC_HUMAN | P24666 | Low molecular weight phosphotyrosine protein phosphatase |
| 227 | ANXA4_HUMAN | P09525 | Annexin A4 |
| 228 | SEPT2_HUMAN | Q15019 | Septin-2 |
| 229 | GSTM3_HUMAN | P21266 | Glutathione 5-transferase Mu 3 |
| 230 | BRK1_HUMAN | Q8WUW1 | Protein BRICK1 |
| 231 | PSB2_HUMAN | P49721 | Proteasome subunit beta type-2 |
| 232 | UBE2K_HUMAN | P61086 | Ubiquitin-conjugating enzyme E2 K |
| 233 | S10A1_HUMAN | P23297 | Protein S100-A1 |
| 234 | MAP1A_HUMAN | P78559 | Microtubule-associated protein 1A |
| 235 | GSTM1_HUMAN | P09488 | Glutathione S-transferase Mu 1 |
| 236 | MMP24_HUMAN | Q9Y5R2 | Matrix metalloproteinase-24 |
| 237 | SIM22_HUMAN | K7EJ46 | Small integral membrane protein 22 |
| 238 | HGNAT_HUMAN | Q68CP4 | Heparan-alpha-glucosaminide N-acetyltransferase |
| 239 | SARG_HUMAN | Q9BW04 | Specifically androgen-regulated gene protein |
| 240 | ABI1_HUMAN | Q8IZP0 | Abl interactor 1 |
| 241 | CF132_HUMAN | Q5T0Z8 | Uncharacterized protein C6orf132 |
| 242 | ARF5_HUMAN | P84085 | ADP-ribosylation factor 5 |
| 243 | IDHC_HUMAN | O75874 | Isocitrate dehydrogenase [NADP] cytoplasmic |
| 244 | GLTP_HUMAN | Q9NZD2 | Glycolipid transfer protein |
| 245 | TPM4_HUMAN | P67936 | Tropomyosin alpha-4 chain |
| 246 | NRAM2_HUMAN | P49281 | Natural resistance-associated macrophage protein 2 |

TABLE 2
Diagnostic potential of differentially expressed proteins.

| | | | Data from | | | Data from | | |
|---|---|---|---|---|---|---|---|---|
| | | | discovery analysis | | | validation analysis | | |
| | | | | #Present | #Present | Validated | Abundance | |
| | Uniprot | Uniprot | | in CTR | in PAT | iBAQ ratio | (ppm of total | #Passed |
| Sequence | Entry Name | Acc. No. | Sensitivity | (of 15) | (of 16) | PAT:CTR | proteome) | criteria |
| 1 | TM256_HUMAN | Q8N2U0 | 94% | 5 | 16 | 140.39 | 4324 | 4 |
| 2 | ADIRF_HUMAN | Q15847 | 81% | 4 | 15 | 18.99 | 369 | 4 |
| 3 | LTOR1_HUMAN | Q6IAA8 | 81% | 4 | 16 | 22.98 | 201 | 4 |
| 4 | PLSL_HUMAN | P13796 | 75% | 14 | 16 | 3.15 | 256 | 4 |
| 5 | RAB2A_HUMAN | P61019 | 75% | 14 | 16 | 3.55 | 1083 | 4 |
| 6 | RAB3B_HUMAN | P20337 | 75% | 15 | 16 | 2.69 | 1138 | 4 |
| 7 | RAB3D_HUMAN | O95716 | 75% | 15 | 16 | 2.24 | 2340 | 4 |
| 8 | RAB7A_HUMAN | P51149 | 75% | 15 | 16 | 3.26 | 2317 | 4 |
| 9 | VATL_HUMAN | P27449 | 75% | 0 | 12 | 3.55 | 861 | 4 |
| 10 | STEA4_HUMAN | Q687X5 | 69% | 14 | 16 | 2.97 | 953 | 4 |
| 11 | PARK7_HUMAN | Q99497 | 69% | 15 | 16 | 1.92 | 957 | 4 |
| 12 | S100P_HUMAN | P25815 | 69% | 14 | 15 | 1.84 | 1351 | 4 |
| 13 | SYTL4_HUMAN | Q96C24 | 69% | 5 | 12 | 3.08 | 91 | 4 |
| 14 | ARL8B_HUMAN | Q9NVJ2 | 63% | 13 | 15 | 2.79 | 49 | 4 |
| 15 | MYCT_HUMAN | Q96QE2 | 63% | 2 | 11 | 2.66 | 100 | 4 |
| 16 | RAB6A_HUMAN | P20340 | 63% | 10 | 16 | 3.36 | 240 | 4 |
| 17 | TSN6_HUMAN | O43657 | 63% | 9 | 16 | 4.03 | 3067 | 4 |
| 18 | CLD10_HUMAN | P78369 | 56% | 7 | 14 | 2.14 | 26 | 4 |
| 19 | CLD2_HUMAN | P57739 | 56% | 2 | 12 | 3.00 | 69 | 4 |
| 20 | CLD3_HUMAN | O15551 | 56% | 1 | 10 | 1.75 | 170 | 4 |
| 21 | GMDS_HUMAN | O60547 | 56% | 2 | 12 | 2.45 | 16 | 4 |
| 22 | GNPI1_HUMAN | P46926 | 56% | 4 | 13 | 15.51 | 44 | 4 |
| 23 | SCRB2_HUMAN | Q14108 | 56% | 15 | 16 | 3.94 | 824 | 4 |
| 24 | MFS12_HUMAN | Q6NUT3 | 56% | 5 | 15 | 8.07 | 65 | 4 |
| 25 | MELPH_HUMAN | Q9BV36 | 56% | 12 | 16 | 2.26 | 151 | 4 |
| 26 | SPRE_HUMAN | P35270 | 56% | 10 | 14 | 2.16 | 114 | 4 |
| 27 | TXD17_HUMAN | Q9BRA2 | 56% | 15 | 16 | 2.35 | 288 | 4 |
| 28 | BDH2_HUMAN | Q9BUT1 | 50% | 14 | 16 | 2.26 | 389 | 4 |
| 29 | CALM_HUMAN | P62158 | 50% | 15 | 16 | 6.30 | 4764 | 4 |
| 30 | CBPQ_HUMAN | Q9Y646 | 50% | 1 | 8 | 5.80 | 51 | 4 |
| 31 | FLOT2_HUMAN | Q14254 | 50% | 14 | 16 | 2.89 | 541 | 4 |
| 32 | LG3BP_HUMAN | Q08380 | 50% | 15 | 16 | 1.99 | 678 | 4 |
| 33 | P2RX4_HUMAN | Q99571 | 50% | 6 | 13 | 2.36 | 76 | 4 |
| 34 | DOP2_HUMAN | Q9Y3R5 | 50% | 12 | 16 | 2.99 | 218 | 4 |
| 35 | S10A6_HUMAN | P06703 | 50% | 15 | 16 | 0.48 | 1853 | 4 |
| 36 | RAB35_HUMAN | Q15286 | 50% | 15 | 16 | 0.57 | 245 | 4 |
| 37 | PP2AA_HUMAN | P67775 | 50% | 11 | 14 | 16.91 | 41 | 4 |
| 38 | DNPH1_HUMAN | O43598 | 44% | 7 | 14 | 2.26 | 117 | 4 |
| 39 | ASAH1_HUMAN | Q13510 | 44% | 15 | 16 | 3.73 | 967 | 4 |
| 40 | CALB1_HUMAN | P05937 | 44% | 15 | 16 | 2.02 | 1907 | 4 |
| 41 | CD59_HUMAN | P13987 | 44% | 15 | 16 | 0.00 | 2 | 4 |
| 42 | CD81_HUMAN | P60033 | 44% | 13 | 15 | 2.61 | 918 | 4 |
| 43 | CY561_HUMAN | P49447 | 44% | 1 | 11 | 24.50 | 86 | 4 |
| 44 | ENOPH_HUMAN | Q9UHY7 | 44% | 1 | 7 | 2.66 | 55 | 4 |
| 45 | GOLP3_HUMAN | Q9H4A6 | 44% | 1 | 15 | 1.86 | 9 | 4 |
| 46 | NICA_HUMAN | Q92542 | 44% | 10 | 16 | 2.37 | 170 | 4 |
| 47 | CPVL_HUMAN | Q9H3G5 | 44% | 1 | 7 | 6.25 | 75 | 4 |
| 48 | LTOR5_HUMAN | O43504 | 44% | 0 | 7 | 2.40 | 192 | 4 |
| 49 | RB27B_HUMAN | O00194 | 44% | 15 | 16 | 2.24 | 1850 | 4 |
| 50 | SCAM2_HUMAN | O15127 | 44% | 0 | 7 | 3.51 | 230 | 4 |
| 51 | SPSY_HUMAN | P52788 | 44% | 8 | 14 | 2.46 | 143 | 4 |
| 52 | SKP1_HUMAN | P63208 | 44% | 10 | 16 | 7.68 | 12 | 4 |
| 53 | TM7S3_HUMAN | Q9NS93 | 44% | 15 | 15 | 4.15 | 198 | 4 |
| 54 | TPD52_HUMAN | P55327 | 44% | 0 | 7 | 13.69 | 34 | 4 |
| 55 | UB2V2_HUMAN | Q15819 | 44% | 11 | 15 | 2.10 | 289 | 4 |
| 56 | UGDH_HUMAN | O60701 | 44% | 13 | 16 | 4.11 | 249 | 4 |
| 57 | ZA2G_HUMAN | P25311 | 44% | 8 | 14 | 2.49 | 942 | 4 |
| 58 | GDPD3_HUMAN | Q7L5L3 | 41% | 2 | 16 | 6.31 | 40 | 4 |
| 59 | EFHD2_HUMAN | Q96C19 | 63% | 13 | 15 | 1.75 | 71 | 3 |
| 60 | RAB14_HUMAN | P61106 | 63% | 15 | 16 | 2.34 | 975 | 3 |
| 61 | NIT2_HUMAN | Q9NQR4 | 63% | 13 | 16 | 1.58 | 393 | 3 |
| 62 | ACTN1_HUMAN | P12814 | 56% | 8 | 12 | 1.33 | 33652 | 3 |
| 63 | MOT5_HUMAN | O15374 | 56% | 0 | 9 | 1.33 | 383 | 3 |
| 64 | RAB12_HUMAN | Q6IQ22 | 56% | 14 | 15 | 1.57 | 22 | 3 |
| 65 | RAB8A_HUMAN | P61006 | 56% | 15 | 16 | 1.24 | 323 | 3 |
| 66 | TM63A_HUMAN | O94886 | 56% | 13 | 16 | 1.71 | 69 | 3 |
| 67 | B2MG_HUMAN | P61769 | 50% | 4 | 9 | 0.20 | 13 | 3 |
| 68 | VA0D1_HUMAN | P61421 | 50% | 14 | 16 | 2.48 | 235 | 3 |
| 69 | LPP1_HUMAN | O14494 | 50% | 15 | 16 | 4.26 | 441 | 3 |
| 70 | GP155_HUMAN | Q7Z3F1 | 50% | 1 | 9 | 11.64 | 9 | 3 |
| 71 | 1433S_HUMAN | P31947 | 50% | 14 | 16 | 0.78 | 215 | 3 |
| 72 | LRSM1_HUMAN | Q6UWE0 | 50% | 0 | 8 | 1.30 | 15 | 3 |
| 73 | DMA_HUMAN | P28067 | 50% | 7 | 7 | 1.67 | 295 | 3 |
| 74 | RAB9A_HUMAN | P51151 | 50% | 3 | 9 | 1.51 | 68 | 3 |
| 75 | SRC8_HUMAN | Q14247 | 50% | 12 | 16 | 0.69 | 101 | 3 |
| 76 | AQP7_HUMAN | O14520 | 44% | 6 | 11 | 2.08 | 248 | 3 |
| 77 | SYUG_HUMAN | O76070 | 44% | 11 | 14 | 2.43 | 183 | 3 |
| 78 | 1433T_HUMAN | P27348 | 44% | 15 | 16 | 0.70 | 175 | 3 |
| 79 | AATC_HUMAN | P17174 | 44% | 15 | 16 | 1.55 | 539 | 3 |
| 80 | CLIC3_HUMAN | O95833 | 44% | 11 | 13 | 1.30 | 156 | 3 |
| 81 | DEST_HUMAN | P60981 | 44% | 15 | 16 | 1.49 | 639 | 3 |
| 82 | RASH_HUMAN | P01112 | 44% | 2 | 8 | 1.60 | 65 | 3 |
| 83 | PTGR2_HUMAN | Q8N8N7 | 44% | 2 | 7 | 1.34 | 94 | 3 |
| 84 | TCPE_HUMAN | P48643 | 44% | 9 | 14 | 1.39 | 13 | 3 |
| 85 | ITIH4_HUMAN | Q14624 | 40% | 6 | 0 | 0.23 | 1 | 3 |
| 86 | AL1A3_HUMAN | P47895 | 38% | 6 | 14 | 4.56 | 97 | 3 |
| 87 | ANXA3_HUMAN | P12429 | 38% | 15 | 16 | 2.80 | 802 | 3 |
| 88 | CLN3_HUMAN | Q13286 | 38% | 1 | 9 | 7.26 | 23 | 3 |
| 89 | CATD_HUMAN | P07339 | 38% | 5 | 16 | 4.37 | 284 | 3 |
| 90 | DDAH1_HUMAN | O94760 | 38% | 15 | 16 | 1.84 | 742 | 3 |
| 91 | NPDC1_HUMAN | Q9NQX5 | 38% | 2 | 8 | 4.88 | 9 | 3 |
| 92 | SAP_HUMAN | P07602 | 38% | 10 | 14 | 2.23 | 115 | 3 |
| 93 | KLK3_HUMAN | P07288 | 38% | 15 | 16 | 2.61 | 783 | 3 |
| 94 | LFG3_HUMAN | Q969X1 | 38% | 15 | 16 | 3.32 | 2745 | 3 |
| 95 | NIBAN_HUMAN | Q9BZQ8 | 38% | 11 | 16 | 3.70 | 210 | 3 |
| 96 | SPNS1_HUMAN | Q9H2V7 | 38% | 1 | 7 | 4.38 | 27 | 3 |
| 97 | LTOR2_HUMAN | Q9Y2Q5 | 38% | 0 | 6 | 4.57 | 226 | 3 |
| 98 | LTOR3_HUMAN | Q9UHA4 | 38% | 0 | 6 | 7.91 | 36 | 3 |
| 99 | SYT7_HUMAN | O43581 | 38% | 7 | 14 | 3.90 | 240 | 3 |
| 100 | T106B_HUMAN | Q9NUM4 | 38% | 0 | 6 | 2.53 | 81 | 3 |
| 101 | MYO5C_HUMAN | Q9NQX4 | 38% | 2 | 11 | 15.50 | 9 | 3 |
| 102 | VAMP2_HUMAN | P63027 | 38% | 14 | 16 | 2.23 | 367 | 3 |
| 103 | VATF_HUMAN | Q16864 | 38% | 7 | 14 | 3.18 | 57 | 3 |
| 104 | AKA7A_HUMAN | O43687 | 31% | 3 | 13 | 2.56 | 24 | 3 |
| 105 | ARSF_HUMAN | P54793 | 31% | 4 | 13 | 5.79 | 75 | 3 |
| 106 | JIP4_HUMAN | O60271 | 31% | 5 | 12 | 2.50 | 34 | 3 |
| 107 | DMBT1_HUMAN | Q9UGM3 | 31% | 7 | 12 | 14.32 | 136 | 3 |
| 108 | GMFB_HUMAN | P60983 | 31% | 1 | 10 | 2.52 | 72 | 3 |
| 109 | FOLH1_HUMAN | Q04609 | 31% | 13 | 16 | 2.69 | 382 | 3 |
| 110 | GSHB_HUMAN | P48637 | 31% | 12 | 14 | 3.05 | 161 | 3 |
| 111 | HPCL1_HUMAN | P37235 | 31% | 13 | 12 | 1.79 | 116 | 3 |
| 112 | MARCS_HUMAN | P29966 | 31% | 15 | 16 | 0.38 | 239 | 3 |
| 113 | NNRE_HUMAN | Q8NCW5 | 31% | 7 | 12 | 2.53 | 226 | 3 |
| 114 | NAPSA_HUMAN | O96009 | 31% | 15 | 16 | 2.83 | 1638 | 3 |
| 115 | AGM1_HUMAN | O95394 | 31% | 8 | 13 | 4.08 | 62 | 3 |
| 116 | AT8A1_HUMAN | Q9Y2Q0 | 31% | 4 | 12 | 3.35 | 50 | 3 |
| 117 | QOR_HUMAN | Q08257 | 31% | 8 | 14 | 2.24 | 175 | 3 |
| 118 | RET5_HUMAN | P82980 | 31% | 14 | 16 | 2.36 | 928 | 3 |
| 119 | SERC2_HUMAN | Q96SA4 | 31% | 15 | 16 | 20.18 | 56 | 3 |
| 120 | S15A1_HUMAN | P46059 | 31% | 0 | 5 | 2.23 | 39 | 3 |
| 121 | SORCN_HUMAN | P30626 | 31% | 14 | 16 | 3.22 | 2828 | 3 |
| 122 | SUIS_HUMAN | P14410 | 31% | 1 | 5 | 2.74 | 4 | 3 |
| 123 | ARPC4_HUMAN | P59998 | 25% | 15 | 16 | 2.35 | 634 | 3 |
| 124 | ARPC5_HUMAN | O15511 | 25% | 15 | 16 | 8.06 | 165 | 3 |
| 125 | APT_HUMAN | P07741 | 25% | 14 | 16 | 2.20 | 356 | 3 |
| 126 | ABRAL_HUMAN | Q9P1F3 | 25% | 5 | 13 | 8.38 | 375 | 3 |
| 127 | IGHA1_HUMAN | P01876 | 25% | 14 | 16 | 4.75 | 593 | 3 |
| 128 | IFM3_HUMAN | Q01628 | 25% | 7 | 15 | 10.13 | 265 | 3 |
| 129 | TRFL_HUMAN | P02788 | 25% | 3 | 11 | 41.74 | 626 | 3 |
| 130 | PNPH_HUMAN | P00491 | 25% | 9 | 16 | 2.39 | 244 | 3 |
| 131 | STXB4_HUMAN | Q6ZWJ1 | 25% | 4 | 10 | 2.23 | 43 | 3 |
| 132 | TSN8_HUMAN | P19075 | 25% | 12 | 16 | 0.51 | 131 | 3 |
| 133 | VPS25_HUMAN | Q9BRG1 | 25% | 14 | 16 | 3.06 | 819 | 3 |
| 134 | VDAC1_HUMAN | P21796 | 25% | 9 | 16 | 6.97 | 116 | 3 |
| 135 | CO6A1_HUMAN | P12109 | 20% | 7 | 2 | 0.00 | 4 | 3 |
| 136 | STAM1_HUMAN | Q92783 | 20% | 12 | 13 | 0.63 | 90 | 3 |
| 137 | CD9_HUMAN | P21926 | 19% | 15 | 16 | 2.03 | 15238 | 3 |
| 138 | FLOT1_HUMAN | O75955 | 19% | 15 | 16 | 1.98 | 589 | 3 |
| 139 | GRAN_HUMAN | P28676 | 19% | 7 | 7 | 2.09 | 723 | 3 |
| 140 | GMPPB_HUMAN | Q9Y5P6 | 19% | 5 | 14 | 2.40 | 71 | 3 |
| 141 | PSA7_HUMAN | O14818 | 19% | 13 | 13 | 3.10 | 244 | 3 |
| 142 | RAB18_HUMAN | Q9NP72 | 19% | 13 | 16 | 1.98 | 243 | 3 |
| 143 | VP37C_HUMAN | A5D8V6 | 19% | 15 | 16 | 2.09 | 761 | 3 |
| 144 | STAM2_HUMAN | O75886 | 13% | 14 | 11 | 0.59 | 51 | 3 |
| 145 | GP2_HUMAN | P55259 | 13% | 4 | 11 | 9.07 | 11 | 3 |
| 146 | PLCG2_HUMAN | P16885 | 7% | 14 | 11 | 0.49 | 40 | 3 |
| 147 | LAMP2_HUMAN | P13473 | 6% | 15 | 16 | 2.81 | 1924 | 3 |
| 148 | CRCT1_HUMAN | Q9UGL9 | 0% | 11 | 3 | 0.30 | 21 | 3 |
| 149 | IPSP_HUMAN | P05154 | 0% | 15 | 11 | 0.41 | 80 | 3 |
| 150 | LCK_HUMAN | P06239 | 0% | 7 | 2 | 0.04 | 35 | 3 |
| 151 | LTOR4 | Q0VGL1 | — | — | — | 7.38 | 159 | 3 |
| 152 | SCAM1_HUMAN | O15126 | 56% | 2 | 10 | 3.88 | nd | 2 |
| 153 | FKB1A_HUMAN | P62942 | 56% | 15 | 16 | 1.27 | 559 | 2 |
| 154 | T176A_HUMAN | Q96HP8 | 50% | 2 | 10 | 3.61 | nd | 2 |
| 155 | TYB4_HUMAN | P62328 | 50% | 15 | 16 | 1.14 | 2890 | 2 |
| 156 | HDHD2_HUMAN | Q9H0R4 | 50% | 1 | 16 | 1.16 | 62 | 2 |
| 157 | CDC42_HUMAN | P60953 | 50% | 15 | 16 | 1.20 | 775 | 2 |
| 158 | RAB17_HUMAN | Q9H0T7 | 50% | 10 | 14 | 1.23 | 99 | 2 |
| 159 | CLIC6_HUMAN | Q96NY7 | 50% | 15 | 16 | 1.24 | 217 | 2 |
| 160 | CTL4_HUMAN | Q53GD3 | 50% | 15 | 16 | 1.26 | 1366 | 2 |
| 161 | BLVRB_HUMAN | P30043 | 50% | 15 | 16 | 1.27 | 439 | 2 |
| 162 | RAB10_HUMAN | P61026 | 50% | 15 | 16 | 1.41 | 2078 | 2 |
| 163 | HEBP2_HUMAN | Q9Y5Z4 | 50% | 15 | 11 | 1.46 | 264 | 2 |
| 164 | FABP5_HUMAN | Q01469 | 50% | 9 | 14 | 1.53 | 54 | 2 |
| 165 | SMIM5_HUMAN | Q71RC9 | 50% | 13 | 13 | 1.64 | 3277 | 2 |
| 166 | LAP4A_HUMAN | Q15012 | 50% | 0 | 8 | INF | nd | 2 |
| 167 | PHYD1_HUMAN | Q5SRE7 | 50% | 0 | 8 | INF | nd | 2 |
| 168 | PSA5_HUMAN | P28066 | 50% | 0 | 8 | INF | nd | 2 |
| 169 | CALL3_HUMAN | P27482 | 44% | 4 | 8 | 4.79 | nd | 2 |
| 170 | PSN1_HUMAN | P49768 | 44% | 1 | 7 | 14.95 | nd | 2 |
| 171 | NQO2_HUMAN | P16083 | 44% | 5 | 13 | 1.03 | 70 | 2 |
| 172 | TCTP_HUMAN | P13693 | 44% | 10 | 16 | 1.20 | 244 | 2 |
| 173 | LAMP1_HUMAN | P11279 | 44% | 15 | 16 | 1.38 | 1279 | 2 |
| 174 | CD38_HUMAN | P28907 | 44% | 7 | 13 | 1.53 | 28 | 2 |
| 175 | MTPN_HUMAN | P58546 | 44% | 15 | 16 | INF | nd | 2 |
| 176 | DYL2_HUMAN | Q96FJ2 | 38% | 10 | 14 | 1.95 | 34 | 2 |
| 177 | DHPR_HUMAN | P09417 | 38% | 15 | 16 | 2.02 | 122 | 2 |
| 178 | NADC_HUMAN | Q15274 | 38% | 4 | 10 | 4.34 | 21 | 2 |
| 179 | CC50A_HUMAN | Q9NV96 | 38% | 8 | 10 | 1.26 | 48 | 2 |
| 180 | PMVK_HUMAN | Q15126 | 38% | 5 | 8 | 0.70 | 16 | 2 |
| 181 | IF4H_HUMAN | Q15056 | 31% | 11 | 15 | 1.86 | 155 | 2 |
| 182 | TTYH3_HUMAN | Q9C0H2 | 31% | 15 | 16 | 2.11 | 132 | 2 |
| 183 | NPT2B_HUMAN | O95436 | 31% | 2 | 11 | 2.72 | 9 | 2 |
| 184 | LAC2_HUMAN(+1) | P0CG05 | 31% | 4 | 9 | 3.28 | 31 | 2 |
| 185 | RABP2_HUMAN | P29373 | 31% | 15 | 15 | 3.63 | 389 | 2 |
| 186 | CUTA_HUMAN | O60888 | 31% | 6 | 12 | 4.03 | 104 | 2 |
| 187 | PSA4_HUMAN | P25789 | 31% | 4 | 10 | 6.11 | 13 | 2 |
| 188 | S35F6_HUMAN | Q8N357 | 31% | 3 | 11 | 6.24 | 25 | 2 |
| 189 | HEM2_HUMAN | P13716 | 31% | 10 | 14 | 1.73 | 63 | 2 |
| 190 | DCXR_HUMAN | Q7Z4W1 | 31% | 15 | 16 | 1.58 | 496 | 2 |
| 191 | UPK1A_HUMAN | O00322 | 31% | 15 | 16 | 1.53 | 3427 | 2 |
| 192 | SPR1A_HUMAN | P35321 | 25% | 8 | 14 | 2.64 | 177 | 2 |
| 193 | ZN185_HUMAN | O15231 | 25% | 5 | 10 | 4.49 | 1 | 2 |
| 194 | TMM8A_HUMAN | Q9HCN3 | 25% | 1 | 7 | 6.47 | 6 | 2 |
| 195 | PCYOX_HUMAN | Q9UHG3 | 25% | 1 | 13 | 8.22 | 24 | 2 |
| 196 | LYSC_HUMAN | P61626 | 25% | 10 | 16 | 23.11 | 1449 | 2 |
| 197 | PALM_HUMAN | O75781 | 19% | 2 | 9 | 3.70 | 3 | 2 |
| 198 | CEAM6_HUMAN | P40199 | 19% | 5 | 8 | 5.05 | 352 | 2 |
| 199 | SC5A1_HUMAN | P13866 | 19% | 15 | 16 | 1.75 | 472 | 2 |
| 200 | PTGR1_HUMAN | Q14914 | 13% | 15 | 16 | 1.57 | 1125 | 2 |
| 201 | S10A9_HUMAN | P06702 | 13% | 10 | 14 | 1.31 | 1928 | 2 |
| 202 | MILK1_HUMAN | Q8N3F8 | 6% | 4 | 6 | 3.41 | 1 | 2 |
| 203 | ABHEB_HUMAN | Q96IU4 | 6% | 14 | 16 | 0.93 | 652 | 2 |
| 204 | AQP2_HUMAN | P41181 | 6% | 15 | 16 | 1.59 | 6762 | 2 |
| 205 | GSTP1_HUMAN | P09211 | 6% | 15 | 16 | 1.71 | 2655 | 2 |
| 206 | ZDHC1_HUMAN | Q8WTX9 | 0% | 15 | 15 | 0.57 | 175 | 2 |
| 207 | RAB8B_HUMAN | Q92930 | 56% | 14 | 16 | 1.30 | nd | 1 |
| 208 | TMPS2_HUMAN | O15393 | 44% | 15 | 16 | 1.16 | nd | 1 |
| 209 | RAB1B_HUMAN | Q9H0U4 | 44% | 15 | 16 | 1.35 | nd | 1 |
| 210 | RAB1A_HUMAN | P62820 | 44% | 15 | 16 | 1.47 | nd | 1 |
| 211 | RAB43_HUMAN | Q86YS6 | 38% | 2 | 8 | 7.56 | nd | 1 |
| 212 | SNG2_HUMAN | O43760 | 38% | 1 | 6 | 8.43 | nd | 1 |
| 213 | 2B1F_HUMAN | P01911 | 38% | 1 | 10 | 9.13 | nd | 1 |
| 214 | NCUG1_HUMAN | Q8WWB7 | 38% | 1 | 6 | 23.59 | nd | 1 |
| 215 | CAB39_HUMAN | Q9Y376 | 38% | 15 | 16 | 0.89 | 1074 | 1 |
| 216 | DYN2_HUMAN | P50570 | 38% | 13 | 16 | 0.98 | 96 | 1 |
| 217 | C42S2_HUMAN | Q9NRR3 | 38% | 8 | 14 | 1.20 | 775 | 1 |
| 218 | FRIH_HUMAN | P02794 | 38% | 11 | 14 | 1.61 | 655 | 1 |
| 219 | S35F2_HUMAN | Q8IXU6 | 38% | 0 | 6 | INF | nd | 1 |
| 220 | PNKD_HUMAN | Q8N490 | 33% | 10 | 6 | 1.52 | 9 | 1 |
| 221 | CATZ_HUMAN | Q9UBR2 | 31% | 5 | 10 | 2.52 | nd | 1 |
| 222 | TBB2B_HUMAN | Q9BVA1 | 31% | 2 | 9 | 7.48 | nd | 1 |
| 223 | TSTD1_HUMAN | Q8NFU3 | 31% | 11 | 15 | 0.93 | 132 | 1 |
| 224 | MK01_HUMAN | P28482 | 31% | 9 | 16 | 1.01 | 54 | 1 |
| 225 | ADHX_HUMAN | P11766 | 31% | 11 | 16 | 1.12 | 178 | 1 |
| 226 | PPAC_HUMAN | P24666 | 31% | 7 | 15 | 1.31 | 52 | 1 |
| 227 | ANXA4_HUMAN | P09525 | 31% | 15 | 16 | 1.36 | 1971 | 1 |
| 228 | SEPT2_HUMAN | Q15019 | 31% | 14 | 16 | 1.44 | 92 | 1 |
| 229 | GSTM3_HUMAN | P21266 | 31% | 15 | 16 | 1.46 | 781 | 1 |
| 230 | BRK1_HUMAN | Q8WUW1 | 31% | 1 | 8 | 1.48 | 41 | 1 |
| 231 | PSB2_HUMAN | P49721 | 31% | 0 | 5 | INF | nd | 1 |
| 232 | UBE2K_HUMAN | P61086 | 31% | 0 | 5 | INF | nd | 1 |
| 233 | S10A1_HUMAN | P23297 | 25% | 6 | 11 | 2.46 | nd | 1 |
| 234 | MAP1A_HUMAN | P78559 | 25% | 1 | 7 | 4.87 | nd | 1 |
| 235 | GSTM1_HUMAN | P09488 | 25% | 10 | 14 | 1.34 | 381 | 1 |
| 236 | MMP24_HUMAN | Q9Y5R2 | 20% | 14 | 5 | 0.39 | nd | 1 |
| 237 | SIM22_HUMAN | K7EJ46 | 19% | 10 | 14 | 2.05 | nd | 1 |
| 238 | HGNAT_HUMAN | Q68CP4 | 19% | 3 | 16 | 4.28 | nd | 1 |
| 239 | SARG_HUMAN | Q9BW04 | 19% | 15 | 16 | 1.06 | 335 | 1 |
| 240 | ABI1_HUMAN | Q8IZP0 | 19% | 15 | 16 | 1.09 | 190 | 1 |
| 241 | CF132_HUMAN | Q5T0Z8 | 19% | 4 | 8 | 1.16 | 8 | 1 |
| 242 | ARF5_HUMAN | P84085 | 13% | 6 | 12 | 0.91 | 56 | 1 |
| 243 | IDHC_HUMAN | O75874 | 13% | 15 | 16 | 1.22 | 2420 | 1 |
| 244 | GLTP_HUMAN | Q9NZD2 | 13% | 14 | 10 | 1.48 | 167 | 1 |
| 245 | TPM4_HUMAN | P67936 | 6% | 9 | 14 | 2.47 | nd | 1 |
| 246 | NRAM2_HUMAN | P49281 | 6% | 10 | 16 | 1.63 | nd | 0 |

*CD59 found in all samples, but only in CTR in validation analysis
**Lactotransferrin found exclusively in fresh (2nd) urine
***LAMTOR4 only found validation analysis, not in discovery analysis
Column: Validated iBAQ ratio PAT:CTR
Bold = found significantly altered (p < 0.05) in PAT in validation analysis
Normal = found in validation analysis (p > 0.05), (iBAQ ratio)
Italics = not found in validation analysis, values from discovery analysis (TOP3TIC ratio)
INF = Infinite; only found in PAT samples
Ratios of <1 indicate that those proteins have a reduced level in urinary exosomes from prostate cancer samples compared to a control level.
Column: Abundance
Abundance: for PAT:CTR > 1.0; total detected protein amount in PAT, for PAT:CTR < 1.0; total detected protein amount in CTR
nd = not detected

TABLE 3
Individual patient sample diagnostic calls associated with the most promising markers. A positive
diagnostic call (expression above diagnostic threshold at 100% specificity) is indicated by ‘1’.

| Sequence | Sensitivity | P1 | P2 | P3 | P4 | P5 | P6 | P7 | P8 | P9 | P10 | P12 | P13 | P14 | P15 | P16 | P17 |
|---|---|---|---|---|---|---|---|---|---|---|---|---|---|---|---|---|---|
| 1 | 94% | 1 | 1 | 1 | 1 | 1 | 1 | 1 | 1 | 1 | 1 | 1 | 1 | 0 | 1 | 1 | 1 |
| 2 | 81% | 1 | 1 | 1 | 1 | 1 | 1 | 1 | 0 | 1 | 1 | 1 | 0 | 0 | 1 | 1 | 1 |
| 3 | 81% | 1 | 1 | 0 | 1 | 1 | 1 | 1 | 1 | 1 | 0 | 1 | 1 | 1 | 1 | 0 | 1 |
| 4 | 75% | 1 | 1 | 0 | 1 | 1 | 0 | 1 | 1 | 1 | 1 | 1 | 0 | 0 | 1 | 1 | 1 |
| 5 | 75% | 1 | 1 | 1 | 1 | 0 | 0 | 1 | 1 | 1 | 1 | 1 | 0 | 0 | 1 | 1 | 1 |
| 6 | 75% | 1 | 1 | 1 | 1 | 0 | 0 | 1 | 1 | 0 | 1 | 1 | 1 | 0 | 1 | 1 | 1 |
| 7 | 75% | 1 | 1 | 1 | 1 | 0 | 1 | 1 | 1 | 0 | 0 | 1 | 1 | 0 | 1 | 1 | 1 |
| 8 | 75% | 1 | 1 | 1 | 1 | 0 | 0 | 1 | 1 | 1 | 1 | 1 | 0 | 0 | 1 | 1 | 1 |
| 9 | 75% | 1 | 1 | 1 | 1 | 1 | 1 | 1 | 0 | 1 | 1 | 1 | 0 | 1 | 0 | 0 | 1 |
| 10 | 69% | 1 | 1 | 1 | 1 | 0 | 1 | 1 | 1 | 1 | 0 | 1 | 0 | 0 | 1 | 1 | 0 |
| 11 | 69% | 1 | 1 | 1 | 1 | 1 | 0 | 0 | 1 | 0 | 1 | 1 | 1 | 0 | 1 | 1 | 0 |
| 12 | 69% | 1 | 1 | 0 | 1 | 1 | 0 | 1 | 1 | 0 | 1 | 1 | 1 | 0 | 1 | 1 | 0 |
| 13 | 69% | 0 | 0 | 1 | 1 | 1 | 1 | 1 | 1 | 0 | 1 | 1 | 0 | 1 | 1 | 1 | 0 |
| 14 | 63% | 1 | 1 | 0 | 1 | 0 | 0 | 1 | 1 | 1 | 1 | 1 | 0 | 0 | 1 | 0 | 1 |
| 15 | 63% | 1 | 1 | 1 | 0 | 1 | 0 | 1 | 0 | 1 | 1 | 1 | 1 | 0 | 0 | 0 | 1 |
| 16 | 63% | 1 | 1 | 1 | 1 | 0 | 1 | 0 | 1 | 0 | 0 | 0 | 1 | 0 | 1 | 1 | 1 |
| 17 | 63% | 1 | 1 | 1 | 1 | 0 | 0 | 1 | 0 | 0 | 1 | 1 | 1 | 0 | 1 | 1 | 0 |
| 18 | 56% | 1 | 1 | 0 | 1 | 1 | 0 | 1 | 1 | 0 | 0 | 1 | 0 | 0 | 1 | 0 | 1 |
| 19 | 56% | 1 | 1 | 1 | 1 | 0 | 1 | 1 | 0 | 1 | 0 | 1 | 0 | 0 | 1 | 0 | 0 |
| 20 | 56% | 1 | 0 | 0 | 1 | 1 | 0 | 0 | 1 | 0 | 1 | 1 | 0 | 1 | 1 | 1 | 0 |
| 21 | 56% | 1 | 1 | 0 | 1 | 1 | 0 | 0 | 0 | 1 | 1 | 1 | 0 | 0 | 1 | 1 | 0 |
| 22 | 56% | 1 | 0 | 0 | 1 | 0 | 0 | 1 | 1 | 0 | 1 | 1 | 1 | 0 | 1 | 1 | 0 |
| 23 | 56% | 1 | 1 | 1 | 1 | 0 | 1 | 1 | 0 | 1 | 0 | 1 | 0 | 0 | 0 | 0 | 1 |
| 24 | 56% | 1 | 1 | 0 | 0 | 0 | 0 | 1 | 1 | 1 | 0 | 1 | 0 | 0 | 1 | 1 | 1 |
| 25 | 56% | 1 | 1 | 0 | 1 | 0 | 0 | 0 | 1 | 0 | 1 | 1 | 1 | 0 | 1 | 1 | 0 |
| 26 | 56% | 1 | 1 | 0 | 1 | 0 | 0 | 1 | 1 | 1 | 1 | 0 | 0 | 0 | 1 | 1 | 0 |
| 27 | 56% | 1 | 1 | 1 | 1 | 0 | 1 | 0 | 0 | 1 | 0 | 1 | 0 | 0 | 0 | 1 | 1 |
| 28 | 50% | 1 | 1 | 0 | 1 | 0 | 1 | 0 | 1 | 1 | 1 | 0 | 0 | 0 | 1 | 0 | 0 |
| 29 | 50% | 1 | 1 | 0 | 1 | 0 | 0 | 0 | 0 | 1 | 0 | 1 | 0 | 0 | 1 | 1 | 1 |
| 30 | 50% | 0 | 1 | 1 | 1 | 0 | 0 | 1 | 1 | 1 | 1 | 0 | 0 | 0 | 1 | 0 | 0 |
| 31 | 50% | 0 | 1 | 0 | 1 | 0 | 0 | 0 | 1 | 0 | 1 | 1 | 1 | 0 | 1 | 1 | 0 |
| 32 | 50% | 1 | 1 | 1 | 0 | 0 | 0 | 0 | 0 | 1 | 1 | 1 | 0 | 0 | 1 | 0 | 1 |
| 33 | 50% | 1 | 1 | 1 | 0 | 0 | 0 | 1 | 1 | 1 | 1 | 1 | 0 | 0 | 0 | 0 | 0 |
| 34 | 50% | 1 | 1 | 0 | 1 | 0 | 0 | 0 | 1 | 0 | 1 | 0 | 1 | 0 | 1 | 1 | 0 |
| 35 | 50% | 1 | 0 | 0 | 1 | 1 | 1 | 1 | 0 | 1 | 0 | 0 | 0 | 1 | 1 | 0 | 0 |
| 36 | 50% | 1 | 1 | 0 | 0 | 0 | 1 | 0 | 0 | 1 | 1 | 1 | 0 | 0 | 1 | 1 | 0 |
| 37 | 50% | 1 | 0 | 1 | 1 | 0 | 0 | 0 | 1 | 0 | 0 | 0 | 1 | 0 | 1 | 1 | 1 |
| 38 | 44% | 1 | 1 | 0 | 1 | 0 | 1 | 0 | 1 | 1 | 0 | 0 | 0 | 0 | 1 | 0 | 0 |
| 39 | 44% | 0 | 1 | 0 | 1 | 0 | 0 | 1 | 1 | 0 | 0 | 0 | 0 | 0 | 1 | 1 | 1 |
| 40 | 44% | 1 | 1 | 0 | 1 | 0 | 0 | 0 | 1 | 0 | 0 | 0 | 0 | 0 | 1 | 1 | 1 |
| 41 | 44% | 1 | 1 | 1 | 1 | 0 | 0 | 0 | 0 | 0 | 0 | 1 | 0 | 0 | 0 | 1 | 1 |
| 42 | 44% | 1 | 0 | 0 | 1 | 0 | 1 | 0 | 1 | 0 | 0 | 0 | 0 | 0 | 1 | 1 | 1 |
| 43 | 44% | 1 | 1 | 0 | 1 | 1 | 0 | 0 | 0 | 0 | 0 | 1 | 0 | 0 | 0 | 1 | 1 |
| 44 | 44% | 1 | 1 | 0 | 1 | 0 | 0 | 0 | 0 | 1 | 0 | 1 | 0 | 0 | 1 | 1 | 0 |
| 45 | 44% | 1 | 1 | 0 | 1 | 0 | 0 | 0 | 1 | 0 | 0 | 0 | 1 | 0 | 1 | 1 | 0 |
| 46 | 44% | 1 | 1 | 0 | 1 | 0 | 0 | 0 | 0 | 1 | 0 | 1 | 0 | 0 | 1 | 0 | 1 |
| 47 | 44% | 1 | 1 | 0 | 0 | 0 | 0 | 0 | 1 | 1 | 0 | 1 | 0 | 0 | 1 | 1 | 0 |
| 48 | 44% | 0 | 0 | 0 | 1 | 0 | 0 | 0 | 1 | 1 | 0 | 1 | 1 | 0 | 1 | 1 | 0 |
| 49 | 44% | 1 | 1 | 0 | 0 | 1 | 0 | 0 | 0 | 0 | 0 | 1 | 0 | 0 | 1 | 1 | 1 |
| 50 | 44% | 1 | 1 | 0 | 0 | 0 | 0 | 0 | 1 | 0 | 1 | 0 | 0 | 0 | 1 | 1 | 1 |
| 51 | 44% | 1 | 0 | 0 | 1 | 0 | 0 | 0 | 1 | 0 | 0 | 1 | 1 | 0 | 1 | 1 | 0 |
| 52 | 44% | 1 | 1 | 1 | 0 | 0 | 0 | 0 | 1 | 1 | 0 | 1 | 0 | 0 | 0 | 0 | 1 |
| 53 | 44% | 0 | 1 | 0 | 0 | 1 | 0 | 1 | 0 | 0 | 1 | 1 | 0 | 0 | 1 | 1 | 0 |
| 54 | 44% | 0 | 1 | 0 | 1 | 0 | 0 | 1 | 0 | 1 | 1 | 1 | 0 | 0 | 0 | 1 | 0 |
| 55 | 44% | 1 | 0 | 0 | 1 | 0 | 0 | 1 | 0 | 0 | 1 | 1 | 0 | 0 | 1 | 1 | 0 |
| 56 | 44% | 0 | 0 | 0 | 1 | 0 | 0 | 1 | 0 | 0 | 1 | 1 | 1 | 1 | 0 | 1 | 0 |
| 57 | 44% | 0 | 1 | 0 | 1 | 1 | 0 | 0 | 1 | 0 | 1 | 1 | 0 | 0 | 1 | 0 | 0 |
| 58 | 41% | 1 | 1 | 1 | 0 | 0 | 0 | 0 | 0 | 1 | 0 | 1 | 0 | 0 | 1 | 0 | 1 |

TABLE 4
Differentially expressed proteins from urinary exosomes which
were also previously identified in exosomes of the PC-3 cell line.
Proteins in bold are included in the focus list of Table 3.

| Uniprot | |
|---|---|
| Ace. No. | Full Protein Name |
| P31947 | 14-3-3 protein sigma |
| P27348 | 14-3-3 protein theta |
| P59998 | Actin-related protein 2/3 complex subunit 4 |
| O15511 | Actin-related protein 2/3 complex subunit 5 |
| Q9NVJ2 | ADP-ribosylation factor-like protein 8B |
| P12429 | Annexin A3 |
| P61769 | Beta-2-microglobulin |
| Q9Y376 | Calcium-binding protein 39 |
| P62158 | Calmodulin |
| P60033 | CD81 antigen |
| P21926 | CD9 antigen |
| O15551 | Claudin-3 |
| P60981 | Destrin |
| P02794 | Ferritin heavy chain |
| O75955 | Flotillin-1 |
| P29966 | Myristoylated alanine-rich C-kinase substrate |
| P13796 | Plastin-2 |
| Q99497 | Protein DJ-1 |
| P61026 | Ras-related protein Rab-10 |
| Q6IQ22 | Ras-related protein Rab-12 |
| P61106 | Ras-related protein Rab-14 |
| P62820 | Ras-related protein Rab-1A |
| Q9H0U4 | Ras-related protein Rab-1B |
| P51149 | Ras-related protein Rab-7a |
| P61006 | Ras-related protein Rab-8A |
| Q92930 | Ras-related protein Rab-8B |
| Q15019 | Septin-2 |
| P13693 | Translationally-controlled tumor protein |
| P63027 | Vesicle-associated membrane protein 2 |

TABLE 5
Examples of biomarker multiplexing for improved diagnostic accuracy. A positive diagnostic
call (expression above diagnostic threshold at 100% specificity) is indicated by ‘1’.

| Sequence | P1 | P2 | P3 | P4 | P5 | P6 | P7 | P8 | P9 | P10 | P12 | P13 | P14 | P15 | P16 | P17 |
|---|---|---|---|---|---|---|---|---|---|---|---|---|---|---|---|---|
| 1 | 1 | 1 | 1 | 1 | 1 | 1 | 1 | 1 | 1 | 1 | 1 | 1 | 0 | 1 | 1 | 1 |
| 3 | 1 | 1 | 0 | 1 | 1 | 1 | 1 | 1 | 1 | 0 | 1 | 1 | 1 | 1 | 0 | 1 |
| 1 | 1 | 1 | 1 | 1 | 1 | 1 | 1 | 1 | 1 | 1 | 1 | 1 | 0 | 1 | 1 | 1 |
| 9 | 1 | 1 | 1 | 1 | 1 | 1 | 1 | 0 | 1 | 1 | 1 | 0 | 1 | 0 | 0 | 1 |
| 1 | 1 | 1 | 1 | 1 | 1 | 1 | 1 | 1 | 1 | 1 | 1 | 1 | 0 | 1 | 1 | 1 |
| 13 | 0 | 0 | 1 | 1 | 1 | 1 | 1 | 1 | 0 | 1 | 1 | 0 | 1 | 1 | 1 | 0 |
| 1 | 1 | 1 | 1 | 1 | 1 | 1 | 1 | 1 | 1 | 1 | 1 | 1 | 0 | 1 | 1 | 1 |
| 20 | 1 | 0 | 0 | 1 | 1 | 0 | 0 | 1 | 0 | 1 | 1 | 0 | 1 | 1 | 1 | 0 |
| 1 | 1 | 1 | 1 | 1 | 1 | 1 | 1 | 1 | 1 | 1 | 1 | 1 | 0 | 1 | 1 | 1 |
| 35 | 1 | 0 | 0 | 1 | 1 | 1 | 1 | 0 | 1 | 0 | 0 | 0 | 1 | 1 | 0 | 0 |
| 1 | 1 | 1 | 1 | 1 | 1 | 1 | 1 | 1 | 1 | 1 | 1 | 1 | 0 | 1 | 1 | 1 |
| 56 | 0 | 0 | 0 | 1 | 0 | 0 | 1 | 0 | 0 | 1 | 1 | 1 | 1 | 0 | 1 | 0 |
| 2 | 1 | 1 | 1 | 1 | 1 | 1 | 1 | 0 | 1 | 1 | 1 | 0 | 0 | 1 | 1 | 1 |
| 3 | 1 | 1 | 0 | 1 | 1 | 1 | 1 | 1 | 1 | 0 | 1 | 1 | 1 | 1 | 0 | 1 |
| 3 | 1 | 1 | 0 | 1 | 1 | 1 | 1 | 1 | 1 | 0 | 1 | 1 | 1 | 1 | 0 | 1 |
| 5 | 1 | 1 | 1 | 1 | 0 | 0 | 1 | 1 | 0 | 1 | 1 | 1 | 0 | 1 | 1 | 1 |
| 3 | 1 | 1 | 0 | 1 | 1 | 1 | 1 | 1 | 1 | 0 | 1 | 1 | 1 | 1 | 0 | 1 |
| 6 | 1 | 1 | 1 | 1 | 0 | 0 | 1 | 1 | 0 | 1 | 1 | 1 | 0 | 1 | 1 | 1 |
| 3 | 1 | 1 | 0 | 1 | 1 | 1 | 1 | 1 | 1 | 0 | 1 | 1 | 1 | 1 | 0 | 1 |
| 8 | 1 | 1 | 1 | 1 | 0 | 0 | 1 | 1 | 1 | 1 | 1 | 0 | 0 | 1 | 1 | 1 |
| 3 | 1 | 1 | 0 | 1 | 1 | 1 | 1 | 1 | 1 | 0 | 1 | 1 | 1 | 1 | 0 | 1 |
| 11 | 1 | 1 | 1 | 1 | 1 | 0 | 0 | 1 | 0 | 1 | 1 | 1 | 0 | 1 | 1 | 0 |
| 3 | 1 | 1 | 0 | 1 | 1 | 1 | 1 | 1 | 1 | 0 | 1 | 1 | 1 | 1 | 0 | 1 |
| 13 | 0 | 0 | 1 | 1 | 1 | 1 | 1 | 1 | 0 | 1 | 1 | 0 | 1 | 1 | 1 | 0 |
| 3 | 1 | 1 | 0 | 1 | 1 | 1 | 1 | 1 | 1 | 0 | 1 | 1 | 1 | 1 | 0 | 1 |
| 17 | 1 | 1 | 1 | 1 | 0 | 0 | 1 | 0 | 0 | 1 | 1 | 1 | 0 | 1 | 1 | 0 |
| 9 | 1 | 1 | 1 | 1 | 1 | 1 | 1 | 0 | 1 | 1 | 1 | 0 | 1 | 0 | 0 | 1 |
| 6 | 1 | 1 | 1 | 1 | 0 | 0 | 1 | 1 | 0 | 1 | 1 | 1 | 0 | 1 | 1 | 1 |
| 9 | 1 | 1 | 1 | 1 | 1 | 1 | 1 | 0 | 1 | 1 | 1 | 0 | 1 | 0 | 0 | 1 |
| 7 | 1 | 1 | 1 | 1 | 0 | 1 | 1 | 1 | 0 | 0 | 1 | 1 | 0 | 1 | 1 | 1 |
| 9 | 1 | 1 | 1 | 1 | 1 | 1 | 1 | 0 | 1 | 1 | 1 | 0 | 1 | 0 | 0 | 1 |
| 11 | 1 | 1 | 1 | 1 | 1 | 0 | 0 | 1 | 0 | 1 | 1 | 1 | 0 | 1 | 1 | 0 |
| 9 | 1 | 1 | 1 | 1 | 1 | 1 | 1 | 0 | 1 | 1 | 1 | 0 | 1 | 0 | 0 | 1 |
| 12 | 1 | 1 | 0 | 1 | 1 | 0 | 1 | 1 | 0 | 1 | 1 | 1 | 0 | 1 | 1 | 0 |
| 13 | 0 | 0 | 1 | 1 | 1 | 1 | 1 | 1 | 0 | 1 | 1 | 0 | 1 | 1 | 1 | 0 |
| 15 | 1 | 1 | 1 | 0 | 1 | 0 | 1 | 0 | 1 | 1 | 1 | 1 | 0 | 0 | 0 | 1 |
| 3 | 1 | 1 | 0 | 1 | 1 | 1 | 1 | 1 | 1 | 0 | 1 | 1 | 1 | 1 | 0 | 1 |
| 4 | 1 | 1 | 0 | 1 | 1 | 0 | 1 | 1 | 1 | 1 | 1 | 0 | 0 | 1 | 1 | 1 |
| 10 | 1 | 1 | 1 | 1 | 0 | 1 | 1 | 1 | 1 | 0 | 1 | 0 | 0 | 1 | 1 | 0 |
| 3 | 1 | 1 | 0 | 1 | 1 | 1 | 1 | 1 | 1 | 0 | 1 | 1 | 1 | 1 | 0 | 1 |
| 14 | 1 | 1 | 0 | 1 | 0 | 0 | 1 | 1 | 1 | 1 | 1 | 0 | 0 | 1 | 0 | 1 |
| 16 | 1 | 1 | 1 | 1 | 0 | 1 | 0 | 1 | 0 | 0 | 0 | 1 | 0 | 1 | 1 | 1 |
| 3 | 1 | 1 | 0 | 1 | 1 | 1 | 1 | 1 | 1 | 0 | 1 | 1 | 1 | 1 | 0 | 1 |
| 10 | 1 | 1 | 1 | 1 | 0 | 1 | 1 | 1 | 1 | 0 | 1 | 0 | 0 | 1 | 1 | 0 |
| 12 | 1 | 1 | 0 | 1 | 1 | 0 | 1 | 1 | 0 | 1 | 1 | 1 | 0 | 1 | 1 | 0 |
| 1 | 1 | 1 | 1 | 1 | 1 | 1 | 1 | 1 | 1 | 1 | 1 | 1 | 0 | 1 | 1 | 1 |
| 2 | 1 | 1 | 1 | 1 | 1 | 1 | 1 | 0 | 1 | 1 | 1 | 0 | 0 | 1 | 1 | 1 |
| 3 | 1 | 1 | 0 | 1 | 1 | 1 | 1 | 1 | 1 | 0 | 1 | 1 | 1 | 1 | 0 | 1 |
| 9 | 1 | 1 | 1 | 1 | 1 | 1 | 1 | 0 | 1 | 1 | 1 | 0 | 1 | 0 | 0 | 1 |

TABLE 6
Diagnostic properties of markers at diagnostic thresholds set to
optimize the combination of sensitivity and specificity

| | | | SEN + |
|---|---|---|---|
| Full protein name | SEN | SPE | SPE |
| Transmembrane protein 256 | 100% | 94% | 194% |
| Adipogenesis regulatory factor | 100% | 81% | 181% |
| Ragulator complex protein LAMTOR1 | 87% | 94% | 180% |
| Vesicle-associated membrane protein 2 | 80% | 100% | 180% |
| V-type proton ATPase 16 kDa proteolipid subunit | 100% | 75% | 175% |
| Acid ceramidase | 93% | 81% | 175% |
| Prenylcysteine oxidase 1 | 93% | 81% | 175% |
| Sorcin | 87% | 88% | 174% |
| Grancalcin | 80% | 94% | 174% |
| Ras-related protein Rab-7a | 80% | 94% | 174% |
| Tetraspanin-6 | 80% | 94% | 174% |
| 3-hydroxybutyrate dehydrogenase type 2 | 87% | 81% | 168% |
| EF-hand domain-containing protein D2 | 87% | 81% | 168% |
| Flotillin-2 | 87% | 81% | 168% |
| Ras-related protein Rab-3D | 80% | 88% | 168% |
| Adenine phosphoribosyltransferase | 73% | 94% | 167% |
| Calmodulin | 73% | 94% | 167% |
| Protein DJ-1 | 73% | 94% | 167% |
| Retinol-binding protein 5 | 73% | 94% | 167% |
| Ubiquitin-conjugating enzyme E2 variant 2 | 73% | 94% | 167% |
| S-phase kinase-associated protein 1 | 67% | 100% | 167% |
| ADP-ribosylation factor-like protein 8B | 93% | 69% | 162% |
| Cytochrome b561 | 93% | 69% | 162% |
| GDP-mannose 4.6 dehydratase | 93% | 69% | 162% |
| Matrix metalloproteinase-24 | 69% | 93% | 162% |
| CD59 glycoprotein | 87% | 75% | 162% |
| Claudin-2 | 87% | 75% | 162% |
| Glutathione synthetase | 87% | 75% | 162% |
| Costars family protein ABRACL | 80% | 81% | 161% |
| Low molecular weight phosphotyrosine protein | 80% | 81% | 161% |
| A-kinase anchor protein 7 isoforms alpha and beta | 80% | 81% | 161% |
| Actin-related protein 2/3 complex subunit 4 | 80% | 81% | 161% |
| 2′-deoxynucleoside 5′-phosphate N-hydrolase 1 | 80% | 81% | 161% |
| Dihydropteridine reductase | 80% | 81% | 161% |
| Ras-related protein Rab-3B | 80% | 81% | 161% |
| Vacuolar protein-sorting-associated protein 25 | 80% | 81% | 161% |
| Cathepsin D | 73% | 88% | 161% |
| L-xylulose reductase | 73% | 88% | 161% |
| Mannose-1-phosphate guanyltransferase beta | 73% | 88% | 161% |
| Napsin-A | 73% | 88% | 161% |
| Purine nucleoside phosphorylase | 73% | 88% | 161% |
| Prostaglandin reductase 1 | 73% | 88% | 161% |
| Ras-related protein Rab-2A | 73% | 88% | 161% |
| Protein S100-P | 73% | 88% | 161% |
| Small integral membrane protein 22 | 73% | 88% | 161% |
| Synaptotagmin-7 | 73% | 88% | 161% |
| Tetraspanin-8 | 73% | 88% | 161% |
| Annexin A4 | 67% | 94% | 160% |
| CD81 antigen | 67% | 94% | 160% |
| Flotillin-1 | 67% | 94% | 160% |
| Interferon-induced transmembrane protein 3 | 67% | 94% | 160% |
| Nicastrin | 67% | 94% | 160% |
| Lipid phosphate phosphohydrolase 1 | 67% | 94% | 160% |
| Ras-related protein Rab-18 | 67% | 94% | 160% |
| Transmembrane protein 63A | 67% | 94% | 160% |
| Transmembrane protease serine 2 | 67% | 94% | 160% |
| UDP-glucose 6-dehydrogenase | 67% | 94% | 160% |

### Example 2

Introduction

In Example 1, we identified 246 proteins differentially expressed in urinary exosomes from prostate cancer patients (16) compared to normal individuals (15) by mass spectrometry (MS). From this analysis, we defined a short list of the most diagnostically promising proteins, demonstrating high individual sensitivity and specificity for prostate cancer.

MS is not yet widely used in clinical laboratories. We have thus investigated the possibility to transfer the identified biomarkers to an immunoassay based analysis platform, which would better integrate into current clinical lab routines. We have obtained commercially available antibodies and ELISA assays for some of the candidate biomarkers. These have been tested in biological samples, and employed to demonstrate the feasibility to transfer the MS-identified biomarkers to an immunoassay platform.

Materials and Methods

Materials

ProteoSilver Plus Silver Stain kit was purchased from Sigma-Aldrich (St. Louis, Mo., USA). Bicinchoninic acid (BCA) protein assay kit was from Pierce (Thermo Scientific, Rockford, Ill., USA). Mini-protean TGX gels and Tranfer-Blot Turbo Transfer Pack were from Bio-Rad (Hercules, Calif., USA). The primary antibodies used for Western blotting were: mouse anti-Flotillin 1 (BD Biosciences), mouse anti-mouse flotillin 2 (BD Biosciences), rabbit anti-Rab3B (Abcam), rabbit anti- LAMTOR1 (Abcam), rabbit anti-TMEM256 (Abcam). HRP-conjugated secondary antibodies were from Jackson Immunoresearch (West Grove, Pa., USA). The DJ-1/PARK? ELISA Kit (CY-9050V2) was from MBL and the Flotillin 2 ELISA kit (ABIN418175) was from Antibodies-online.com

Urine Collection and Exosome Isolation

Urine collection and exosome isolation was performed as described in Example 1 and published in Øverbye A. et al, 2015, Oncotarget. 6(30):30357-76.

Total Protein Quantification

The amount of total protein in exosomes was determined using a BCA assay kit according to the manufacturer's instructions. BSA was used as standard protein.

SDS-PAGE and Silver Staining

Similar amounts of urinary exosomes were mixed with loading buffer, and the samples were run on 4-20% polyacrylamide gels. The gels were stained using ProteoSilver Plus Silver Stain kit following the manufacturer's protocol.

SDS-PAGE and Immunoblotting

Similar amounts of urinary exosomes were solubilised in loading buffer and run on 4-20% gradient TGX gels. The proteins were transferred to PVDF membranes using a Tranfer-Blot Turbo Transfer Pack. Membranes were incubated with the specified primary and secondary antibodies. Blots were visualized with the Amersham™ ECL™ Prime Western blot detection (GE Healthcare, Little Chalfont, UK) on the Universal Hood II Bio-Rad scanner (Bio-Rad, Hercules, Calif., USA).

ELISA Assays

Similar amounts of urinary exosomes were analyzed following the manufacturer's protocol.

Results

Urine was collected and exosomes isolated as previously described in Example 1 and Øverbye A. et al, 2015, Oncotarget 6(30):30357-76. In order to analyze similar amounts of urinary exosomes from the different individuals, the protein amount of exosomes was measured by the BCA assay and/or by the intensity of silver stained samples (data not shown). First, Western blot experiments designed to detect flotillin1, flotillin2, TM256, Rab-3B and LAMTOR1 were performed. In order to identify the amount of exosomes required to detect specific proteins by Western blot, several amounts of exosomes were loaded on gels. As shown in FIG. 3A, relatively low amounts of exosomes were required in order to detect these proteins by Western blot. The intensity of the bands was related to the sample amount/concentration to produce a standard curve as shown in FIG. 3B for flotillin2. Then, similar protein amounts of urinary exosomes from 9 healthy controls and 9 patients were analyzed. In agreement with the mass spectrometry data (Example 1, Øverbye A. et al, 2015, Oncotarget 6(30):30357-76), there were clear differences in the levels of the tested proteins between healthy control males and prostate cancer patients. The patient to control ratio for the different proteins (based on the sum of the individual intensities for each protein in the control and in the patient group) is shown in FIG. 4. Furthermore, analysis of individual Western blot signals for flotillin2 in 16 controls and 16 patients showed 87.5% sensitivity (14/16 patients corrected identified as positive) and 94% specificity (15/16 controls correctly identified as negative) at an optimally set expression threshold (FIG. 5).

As a next step, ELISA assays were performed to validate the Western blot results of two protein markers, flotillin 2 and PARK7 (Protein DJ-1). Since flotillin2 is expected to be located in the exosomal lumen, exosomes solubilized in 0.5% Triton X-100 were used in these experiments. Control experiments showed that the ELISA kit was compatible with this concentration of Triton X-100. Standard curves were created for both protein markers and different amounts of control urinary exosomes were tested (data not shown). Once the amount of urinary exosomes required to detect the proteins with the ELISA kit were calculated, similar amounts of control and patient samples were analyzed. The ELISA assays indicate that levels for both proteins were higher in the prostate cancer samples than in healthy controls (1.5 fold higher for flotillin2, 1.8 fold higher for PARK7), in general agreement with the Western blot and the MS data.

In conclusion, our data support the feasibility of detecting the MS-identified protein biomarkers in urinary exosomes by antibody based detection methods.

1. A method of screening for prostate cancer in a subject, said method comprising
determining the level in a sample of one or more polypeptides selected from the group consisting of:
Transmembrane protein 256, Adipogenesis regulatory factor, Ragulator complex protein LAMTOR1, Plastin-2, Ras-related protein Rab-2A, Ras-related protein Rab-3B, Ras-related protein Rab-3D, Ras-related protein Rab-7a, V-type proton ATPase 16 kDa proteolipid subunit, Metalloreductase STEAP4, Protein DJ-1, Protein S100-P, Synaptotagmin-like protein 4, ADP-ribosylation factor-like protein 8B, Proton myo-inositol cotransporter, Ras-related protein Rab-6A, Tetraspanin-6, Claudin-10, Claudin-2, Claudin-3, GDP-mannose 4.6 dehydratase, Glucosamine-6-phosphate isomerase 1, Lysosome membrane protein 2, Major facilitator superfamily domain-containing protein 12, Melanophilin, Sepiapterin reductase, Thioredoxin domain-containing protein 17, 3-hydroxybutyrate dehydrogenase type 2, Calmodulin, Carboxypeptidase Q, Flotillin-2, Galectin-3-binding protein, P2X purinoceptor 4, Protein dopey-2, Protein S100-A6, Ras-related protein Rab-35, Serine/threonine-protein phosphatase 2A catalytic subunit alpha isoform, 2′-deoxynucleoside 5′-phosphate N-hydrolase 1, Acid ceramidase, Calbindin, CD59 glycoprotein*, CD81 antigen, Cytochrome b561, Enolase-phosphatase E1, Golgi phosphoprotein 3, Nicastrin, Probable serine carboxypeptidase CPVL, Ragulator complex protein LAMTORS, Ras-related protein Rab-27B, Secretory carrier-associated membrane protein 2, Spermine synthase, S-phase kinase-associated protein 1, Transmembrane 7 superfamily member 3, Tumor protein D52, Ubiquitin-conjugating enzyme E2 variant 2, UDP-glucose 6-dehydrogenase, Zinc-alpha-2-glycoprotein, Glycerophosphodiester phosphodiesterase domain-containing protein 3, EF-hand domain-containing protein D2, Ras-related protein Rab-14, Omega-amidase NIT2, Alpha-actinin-1, Monocarboxylate transporter 5, Ras-related protein Rab-12, Ras-related protein Rab-8A, Transmembrane protein 63A, Beta-2-microglobulin, V-type proton ATPase subunit d 1, Lipid phosphate phosphohydrolase 1, Integral membrane protein GPR155, 14-3-3 protein sigma, E3 ubiquitin-protein ligase LRSAM1, HLA class II histocompatibility antigen. DM alpha chain, Ras-related protein Rab-9A, Src substrate cortactin, Aquaporin-7, Gamma-synuclein, 14-3-3 protein theta, Aspartate aminotransferase, cytoplasmic, Chloride intracellular channel protein 3, Destrin, GTPase HRas, Prostaglandin reductase 2, T-complex protein 1 subunit epsilon, Inter-alpha-trypsin inhibitor heavy chain H4, Aldehyde dehydrogenase family 1 member A3, Annexin A3, Battenin, Cathepsin D, N(G).N(G)-dimethylarginine dimethylaminohydrolase 1, Neural proliferation differentiation and control protein 1, Proactivator polypeptide, Prostate-specific antigen, Protein lifeguard 3, Protein Niban, Protein spinster homolog 1, Ragulator complex protein LAMTOR2, Ragulator complex protein LAMTOR3, Synaptotagmin-7, Transmembrane protein 106B, Unconventional myosin-Vc, Vesicle-associated membrane protein 2, V-type proton ATPase subunit F, A-kinase anchor protein 7 isoforms alpha and beta, Arylsulfatase F, C-Jun-amino-terminal kinase-interacting protein 4, Deleted in malignant brain tumors 1 protein, Glia maturation factor beta, Glutamate carboxypeptidase 2, Glutathione synthetase, Hippocalcin-like protein 1, Myristoylated alanine-rich C-kinase substrate, NAD(P)H-hydrate epimerase, Napsin-A, Phosphoacetylglucosamine mutase, Probable phospholipid-transporting ATPase IA, Quinone oxidoreductase, Retinol-binding protein 5, Serine incorporator 2, Solute carrier family 15 member 1, Sorcin, Sucrase-isomaltase. Intestinal, Actin-related protein ⅔ complex subunit 4, Actin-related protein ⅔ complex subunit 5, Adenine phosphoribosyltransferase, Costars family protein ABRACL, Ig alpha-1 chain C region, Interferon-induced transmembrane protein 3, Lactotransferrin, Purine nucleoside phosphorylase, Syntaxin-binding protein 4, Tetraspanin-8, Vacuolar protein-sorting-associated protein 25, Voltage-dependent anion-selective channel protein 1, Collagen alpha-1(VI) chain, Signal transducing adapter molecule 1, CD9 antigen, Flotillin-1, Grancalcin, Mannose-1-phosphate guanyltransferase beta, Proteasome subunit alpha type-7, Ras-related protein Rab-18, Vacuolar protein sorting-associated protein 37C, Signal transducing adapter molecule 2, Pancreatic secretory granule membrane major glycoprotein GP2, 1-phosphatidylinositol 4.5-bisphosphate phosphodiesterase gamma-2, Lysosome-associated membrane glycoprotein 2, Cysteine-rich C-terminal protein 1, Plasma serine protease inhibitor, Tyrosine-protein kinase Lck, Ragulator complex protein LAMTOR4, Secretory carrier-associated membrane protein 1, Peptidyl-prolyl cis-trans isomerase FKBP1A, Transmembrane protein 176A, Thymosin beta-4, Haloacid dehalogenase-like hydrolase domain-containing protein 2, Cell division control protein 42 homolog, Ras-related protein Rab-17, Chloride intracellular channel protein 6, Choline transporter-like protein 4, Flavin reductase (NADPH), Ras-related protein Rab-10, Heme-binding protein 2, Fatty acid-binding protein. epidermal, Small integral membrane protein 5, Lysosomal-associated transmembrane protein 4A, Phytanoyl-CoA dioxygenase domain-containing protein 1, Proteasome subunit alpha type-5, Calmodulin-like protein 3, Presenilin-1, Ribosyldihydronicotinamide dehydrogenase [quinone], Translationally-controlled tumor protein, Lysosome-associated membrane glycoprotein 1, ADP-ribosyl cyclase 1, Myotrophin, Dynein light chain 2. cytoplasmic, Dihydropteridine reductase, Nicotinate-nucleotide pyrophosphorylase [carboxylating], Cell cycle control protein 50A, Phosphomevalonate kinase, Eukaryotic translation initiation factor 4H, Protein tweety homolog 3, Sodium-dependent phosphate transport protein 2B, Ig lambda-2 chain C regions, Cellular retinoic acid-binding protein 2, Protein CutA, Proteasome subunit alpha type-4, Solute carrier family 35 member F6, Delta-aminolevulinic acid dehydratase, L-xylulose reductase, Uroplakin-1 a, Cornifin-A, Zinc finger protein 185, Transmembrane protein 8A, Prenylcysteine oxidase 1, Lysozyme C, Paralemmin-1, Carcinoembryonic antigen-related cell adhesion molecule 6, Sodium/glucose cotransporter 1, Prostaglandin reductase 1, Protein S100-A9, MICAL-like protein 1, Alpha/beta hydrolase domain-containing protein 14B, Aquaporin-2, Glutathione S-transferase P, Probable almitoyltransferase ZDHHC1, Ras-related protein Rab-8B, Transmembrane protease serine 2, Ras-related protein Rab-1 B, Ras-related protein Rab-1A, Ras-related protein Rab-43, Synaptogyrin-2, HLA class II histocompatibility antigen. DRB1-15 beta chain, Lysosomal protein NCU-G1, Calcium-binding protein 39, Dynamin-2, CDC42 small effector protein 2, Ferritin heavy chain, Solute carrier family 35 member F2,Probable hydrolase PNKD, Cathepsin Z, Tubulin beta-2B chain, Thiosulfate sulfurtransferase/rhodanese-like domain-containing protein 1, Mitogen-activated protein kinase 1, Alcohol dehydrogenase class-3, Low molecular weight phosphotyrosine protein phosphatase, Annexin A4, Septin-2, Glutathione S-transferase Mu 3, Protein BRICK1, Proteasome subunit beta type-2, Ubiquitin-conjugating enzyme E2 K, Protein S100-A1, Microtubule-associated protein 1A, Glutathione S-transferase Mu 1, Matrix metalloproteinase-24, Small integral membrane protein 22, Heparan-alpha-glucosaminide N-acetyltransferase, Specifically androgen-regulated gene protein, Abl interactor 1, Uncharacterized protein C6orf132, ADP-ribosylation factor 5, Isocitrate dehydrogenase [NADP] cytoplasmic, Glycolipid transfer protein, Tropomyosin alpha-4 chain and Natural resistance-associated macrophage protein 2;
wherein said sample comprises urinary exosomes and wherein said sample has been obtained from said subject;
wherein an increased level in said sample of one or more of said polypeptides selected from the group consisting of Transmembrane protein 256, Adipogenesis regulatory factor, Ragulator complex protein LAMTOR1, Plastin-2, Ras-related protein Rab-2A, Ras-related protein Rab-3B, Ras-related protein Rab-3D, Ras-related protein Rab-7a, V-type proton ATPase 16 kDa proteolipid subunit, Metalloreductase STEAP4, Protein DJ-1, Protein S100-P, Synaptotagmin-like protein 4, ADP-ribosylation factor-like protein 8B, Proton myo-inositol cotransporter, Ras-related protein Rab-6A, Tetraspanin-6, Claudin-10, Claudin-2, Claudin-3, GDP-mannose 4.6 dehydratase, Glucosamine-6-phosphate isomerase 1, Lysosome membrane protein 2, Major facilitator superfamily domain-containing protein 12, Melanophilin, Sepiapterin reductase, Thioredoxin domain-containing protein 17, 3-hydroxybutyrate dehydrogenase type 2, Calmodulin, Carboxypeptidase Q, Flotillin-2, Galectin-3-binding protein, P2X purinoceptor 4, Protein dopey-2, Serine/threonine-protein phosphatase 2A catalytic subunit alpha isoform, 2′-deoxynucleoside 5′-phosphate N-hydrolase 1, Acid ceramidase, Calbindin, CD81 antigen, Cytochrome b561, Enolase-phosphatase E1, Golgi phosphoprotein 3, Nicastrin, Probable serine carboxypeptidase CPVL, Ragulator complex protein LAMTORS, Ras-related protein Rab-27B, Secretory carrier-associated membrane protein 2, Spermine synthase, S-phase kinase-associated protein 1, Transmembrane 7 superfamily member 3, Tumor protein D52, Ubiquitin-conjugating enzyme E2 variant 2, UDP-glucose 6-dehydrogenase, Zinc-alpha-2-glycoprotein, Glycerophosphodiester phosphodiesterase domain-containing protein 3, EF-hand domain-containing protein D2, Ras-related protein Rab-14, Omega-amidase NIT2, Alpha-actinin-1, Monocarboxylate transporter 5, Ras-related protein Rab-12, Ras-related protein Rab-8A, Transmembrane protein 63A, V-type proton ATPase subunit d 1, Lipid phosphate phosphohydrolase 1, Integral membrane protein GPR155, E3 ubiquitin-protein ligase LRSAM1, HLA class II histocompatibility antigen. DM alpha chain, Ras-related protein Rab-9A, Aquaporin-7, Gamma-synuclein, Aspartate aminotransferase. cytoplasmic, Chloride intracellular channel protein 3, Destrin, GTPase HRas, Prostaglandin reductase 2, T-complex protein 1 subunit epsilon, Aldehyde dehydrogenase family 1 member A3, Annexin A3, Battenin, Cathepsin D, N(G).N(G)-dimethylarginine dimethylaminohydrolase 1, Neural proliferation differentiation and control protein 1, Proactivator polypeptide, Prostate-specific antigen, Protein lifeguard 3, Protein Niban, Protein spinster homolog 1, Ragulator complex protein LAMTOR2, Ragulator complex protein LAMTOR3, Synaptotagmin-7, Transmembrane protein 106B, Unconventional myosin-Vc, Vesicle-associated membrane protein 2, V-type proton ATPase subunit F, A-kinase anchor protein 7 isoforms alpha and beta, Arylsulfatase F, C-Jun-amino-terminal kinase-interacting protein 4, Deleted in malignant brain tumors 1 protein, Glia maturation factor beta, Glutamate carboxypeptidase 2, Glutathione synthetase, Hippocalcin-like protein 1, NAD(P)H-hydrate epimerase, Napsin-A, Phosphoacetylglucosamine mutase, Probable phospholipid-transporting ATPase IA, Quinone oxidoreductase, Retinol-binding protein 5, Serine incorporator 2, Solute carrier family 15 member 1, Sorcin, Sucrase-isomaltase. Intestinal, Actin-related protein ⅔ complex subunit 4, Actin-related protein ⅔ complex subunit 5, Adenine phosphoribosyltransferase, Costars family protein ABRACL, Ig alpha-1 chain C region, Interferon-induced transmembrane protein 3, Lactotransferrin, Purine nucleoside phosphorylase, Syntaxin-binding protein 4, Vacuolar protein-sorting-associated protein 25, Voltage-dependent anion-selective channel protein 1, CD9 antigen, Flotillin-1, Grancalcin, Mannose-1-phosphate guanyltransferase beta, Proteasome subunit alpha type-7, Ras-related protein Rab-18, Vacuolar protein sorting-associated protein 37C, Pancreatic secretory granule membrane major glycoprotein GP2, Lysosome-associated membrane glycoprotein 2, Ragulator complex protein LAMTOR4, Secretory carrier-associated membrane protein 1, Peptidyl-prolyl cis-trans isomerase FKBP1A, Transmembrane protein 176A, Thymosin beta-4, Haloacid dehalogenase-like hydrolase domain-containing protein 2, Cell division control protein 42 homolog, Ras-related protein Rab-17, Chloride intracellular channel protein 6, Choline transporter-like protein 4, Flavin reductase (NADPH), Ras-related protein Rab-10, Heme-binding protein 2, Fatty acid-binding protein. epidermal, Small integral membrane protein 5, Lysosomal-associated transmembrane protein 4A, Phytanoyl-CoA dioxygenase domain-containing protein 1, Proteasome subunit alpha type-5, Calmodulin-like protein 3, Presenilin-1, Ribosyldihydronicotinamide dehydrogenase [quinone], Translationally-controlled tumor protein, Lysosome-associated membrane glycoprotein 1, ADP-ribosyl cyclase 1, Myotrophin, Dynein light chain 2. cytoplasmic, Dihydropteridine reductase, Nicotinate-nucleotide pyrophosphorylase [carboxylating], Cell cycle control protein 50A, Eukaryotic translation initiation factor 4H, Protein tweety homolog 3, Sodium-dependent phosphate transport protein 2B, Ig lambda-2 chain C regions, Cellular retinoic acid-binding protein 2, Protein CutA, Proteasome subunit alpha type-4, Solute carrier family 35 member F6, Delta-aminolevulinic acid dehydratase, L-xylulose reductase, Uroplakin-1a, Cornifin-A, Zinc finger protein 185, Transmembrane protein 8A, Prenylcysteine oxidase 1, Lysozyme C, Paralemmin-1, Carcinoembryonic antigen-related cell adhesion molecule 6, Sodium/glucose cotransporter 1, Prostaglandin reductase 1, Protein S100-A9, MICAL-like protein 1, Aquaporin-2, Glutathione S-transferase P, Ras-related protein Rab-8B, Transmembrane protease serine 2, Ras-related protein Rab-1 B, Ras-related protein Rab-1A, Ras-related protein Rab-43, Synaptogyrin-2, HLA class II histocompatibility antigen. DRB1-15 beta chain, Lysosomal protein NCU-G1, CDC42 small effector protein 2, Ferritin heavy chain, Solute carrier family 35 member F2,Probable hydrolase PNKD, Cathepsin Z, Tubulin beta-2B chain, Mitogen-activated protein kinase 1, Alcohol dehydrogenase class-3, Low molecular weight phosphotyrosine protein phosphatase, Annexin A4, Septin-2, Glutathione S-transferase Mu 3, Protein BRICK1, Proteasome subunit beta type-2, Ubiquitin- conjugating enzyme E2 K, Protein S100-A1, Microtubule-associated protein 1A, Glutathione S-transferase Mu 1, Small integral membrane protein 22, Heparan-alpha-glucosaminide N-acetyltransferase, Specifically androgen-regulated gene protein, Abl interactor 1, Uncharacterized protein C6orf132, Isocitrate dehydrogenase [NADP] cytoplasmic, Glycolipid transfer protein, Tropomyosin alpha-4 chain and Natural resistance-associated macrophage protein 2 in comparison to a control level is indicative of prostate cancer in said subject; and/or
wherein a decreased level in said sample of one or more of said polypeptides selected from the group consisting of Protein S100-A6, Ras-related protein Rab-35, CD59 glycoprotein, Beta-2-microglobulin, 14-3-3 protein sigma, Src substrate cortactin, 14-3-3 protein theta, Inter-alpha-trypsin inhibitor heavy chain H4, Myristoylated alanine-rich C-kinase substrate, Tetraspanin-8, Collagen alpha-1(VI) chain, Signal transducing adapter molecule 1, Signal transducing adapter molecule 2, 1-phosphatidylinositol 4.5-bisphosphate phosphodiesterase gamma-2, Cysteine-rich C-terminal protein 1, Plasma serine protease inhibitor, Tyrosine-protein kinase Lck, Phosphomevalonate kinase, Alpha/beta hydrolase domain-containing protein 14B, Probable almitoyltransferase ZDHHC1, Calcium-binding protein 39, Dynamin-2, Thiosulfate sulfurtransferase/rhodanese-like domain-containing protein 1, Matrix metalloproteinase-24 and ADP-ribosylation factor 5 in comparison to a control level is indicative of prostate cancer in said subject.

2. The method of claim 1, wherein said method comprises determining the level in a sample of one or more polypeptides selected from the group consisting of:
Transmembrane protein 256, Ragulator complex protein LAMTOR1, V-type proton ATPase 16 kDa proteolipid subunit, Synaptotagmin-like protein 4, Claudin-3, Protein S100-A6, UDP-glucose 6-dehydrogenase, Adipogenesis regulatory factor, Ras-related protein Rab-2A, Ras-related protein Rab-3B, Ras-related protein Rab-7a, Protein DJ-1, Tetraspanin-6, Ras-related protein Rab-3D, Protein S100-P, Proton myo-inositol cotransporter, Plastin-2, Metalloreductase STEAP4, ADP-ribosylation factor-like protein 8B, Ras-related protein Rab-6A, Vesicle-associated membrane protein 2, Prenylcysteine oxidase 1, Sorcin and Grancalcin.

3. The method of claim 1, wherein said method comprises determining the level in a sample of one or more polypeptides selected from the group consisting of:
Transmembrane protein 256, Adipogenesis regulatory factor and Ragulator complex protein LAMTOR1.

4. The method of claim 1, wherein said method comprises determining the level of Transmembrane protein 256.

5. The method of claim 1, wherein said method comprises determining the level of more than one of said polypeptides.

6. The method of claim 1, wherein said method comprises determining the level of two, three or four of said polypeptides.

7. The method of claim 1, wherein said method comprises determining the level of Transmembrane protein 256 and Ragulator complex protein LAMTOR1.

8. The method of claim 1, wherein said method is used for diagnosing prostate cancer, for the prognosis of prostate cancer, for monitoring the progression of prostate cancer in a subject, for determining the clinical severity of prostate cancer, for predicting the response of a subject to therapy, or for determining the efficacy of a therapeutic regime being used to treat prostate cancer.

9. The method of claim 1, wherein said method is used for determining the aggressiveness of prostate cancer (e.g. distinguishing between indolent and aggressive cancer).

10. The method of claim 1, wherein said level of said one or more polypeptides is determined by immunoassay.

11. The method of claim 1, wherein said level of said one or more polypeptides is determined by mass spectrometry.

12. The method of claim 1, wherein said subject is a human subject.

13. A kit for the screening of prostate cancer which comprises an agent suitable for determining the level of one or more of the polypeptides as defined in claim 1, or fragments thereof, in a sample.

14. The kit of claim 13, wherein said agent is an antibody.
